(19) United States
(12) Patent Application Publication
(10) Pub. No.: US 2006/0166242 A1
(43) Pub. Date: Jul. 27, 2006

(54) Markers of pre-term labor

(75) Inventors: Craig Pennell, Toronto (CA); Stephen Lye, Toronto (CA)

Correspondence Address: HAMRE, SCHUMANN, MUELLER & LARSON, P.C., P.O. BOX 2902-0902, MINNEAPOLIS, MN, 55402, US

(73) Assignee: Mount Sinai Hospital, Toronto (CA)

(21) Appl. No.: 11/327,124

(22) Filed: Jan. 6, 2006

Related U.S. Application Data
(60) Provisional application No. 60/641,875, filed on Jan. 6, 2005.

(51) Int. Cl. C12Q 1/68; G01N 33/00

(52) U.S. Cl. 435/6; 436/86

(57) ABSTRACT

The invention relates to novel markers of pre-term labor, methods for assessing the status of pre-term labor using the markers, and methods for the diagnosis and therapy of pre-term labor.

### FIELD OF THE INVENTION

The invention relates to novel markers of pre-term labor, methods for assessing pre-term labor using the markers, and methods for the detection, diagnosis, prediction, monitoring, preventing, and therapy of pre-term labor

### BACKGROUND OF THE INVENTION

Threatened pre-term labor occurs in many women during pregnancy and accounts for one third of all antenatal hospital admissions for pregnant women (15). Fortunately most women who present with threatened pre-term labor do not progress to pre-term delivery. Unfortunately the ability to predict the small percentage who will progress to delivery (true pre-term labor) within 7-10 days is poor (19-32), which leads to large numbers of women and their babies being hospitalized and unnecessarily exposed to potentially dangerous side effects associated with tocolytic and glucocorticoid administration. It is therefore of critical importance to develop new non-invasive methods to accurately and reliably diagnose pre-term labor which will 1) provide the means to effectively triage patients and so reduce demands on limited health care resources, and 2) limit the use of existing approaches such as antenatal glucocorticoids, short-term tocolysis, and transfer to tertiary perinatal facilities to those patients whose pre-term birth is imminent.

Currently there is no diagnostic test that will define those women in threatened pre-term labor (T-PTL) who will deliver within the next 7-10 days with both high positive and negative predictive values. A test based on fetal fibronectin (developed by Adeza, Calif.) is widely used in the US and Australia: while it has a high negative predictive value, its positive predictive value is low (˜15%). A further major limitation of fetal fibronectin is that there are many contra-indications to performing this test limiting its use to only approximately 20% of women presenting with threatened pre-term labor.

### SUMMARY OF THE INVENTION

Applicants using micro-array technology, have identified distinct patterns of gene expression in women presenting with threatened pre-term labor who progress to delivery compared to those whose pregnancies continue to term. In particular, it was found that symptomatic women who present with threatened pre-term labor who progress to pre-term delivery (true pre-term labor) have different gene expression profiles in peripheral white blood cells when compared to those women who present with threatened pre-term labor who do not deliver within 48 hours. A test based on this gene expression “signature” will have both a high positive and negative predictive value for premature delivery in women presenting with signs and/or symptoms of pre-term labor. The use of these tests has significant advantages. They will result in a decrease in hospitalization, and administration of glucocorticoid and tocolytic therapy for symptomatic women that are not in true pre-term labor and thereby reduce costs to the health care system.

Thus, Applicants have developed a method for identifying markers associated with threatened pre-term labor that progresses to delivery. Using the method they analyzed samples from patients, and identified novel correlations between the expression of certain markers and threatened pre-term labor that progresses to delivery as well as markers associated with pregnancies that continue to term. The invention therefore provides a set of markers that can distinguish threatened pre-term labor that progresses to delivery. Methods are provided for use of these markers to distinguish between the patient groups, and to determine general courses of treatment.

In an aspect, the invention relates to a method of characterizing a biological sample by detecting or quantitating in the sample one or more polynucleotides extracted from the sample that are characteristic of pre-term labor or onset of pre-term labor the method comprising assaying for differential expression of polynucleotides in the sample. Differential expression of the polynucleotides can be determined by micro-array, hybridization or by amplification of the extracted polynucleotides.

The invention also relates to a method of characterizing or classifying a sample by detecting or quantitating in the sample one or more polypeptides extracted from the sample that are characteristic of pre-term labor or onset of pre-term labor, the method comprising assaying for differential expression of polypeptides in the sample. Differential expression of polypeptides can be assayed using procedures known in the art, including without limitation, separation techniques known in the art, antibody microarrays, or mass spectroscopy of polypeptides extracted from a sample.

An embodiment of the invention is directed to bioinformatic methods for analyzing gene expression data generated from nucleic acid micro-array experiments to identify further biomarker genes from various cell types. Another embodiment of the invention is directed to biomarker genes identified from mammalian (e.g., human, primate) peripheral blood cells at normal and/or abnormal states. The biomarker genes are useful as molecular targets for therapeutics of a disorder or disease in mammals.

The invention contemplates a gene expression “signature” identified using a method of the invention that is associated with delivery within about 48 hours in women presenting with idiopathic threatened pre-term labor. This signature provides a highly sensitive and specific test with both high positive and negative predictive values permitting diagnosis and prediction of birth.

The invention provides gene marker sets that distinguish preterm labor, term labor or onset of pre-term labor and uses therefor. A genetic marker set may comprise a plurality of genes comprising or consisting of at least 5, 10, 15, 20, 25, 30, 35, 40, 45, or 50 of the genes corresponding to the markers listed in Table 2, 3, 4, 5, and/or 6, or SEQ ID Nos. 1 through 232. In certain aspects, the plurality of genes consists of at least 50, 60, 70, 80, 90, 100, 110, 120, 130, 140, 150, 160, 170, 180, 190, 200, 210, 220, 230, 240, 250, 260, 270, 280, 290, or 300 of the gene markers listed in Table 2, 3, 4, 5, and/or 6, or SEQ ID Nos. 1 through 232. In an aspect, the gene marker sets comprise gene clusters which may be represented by dendograms (see FIGS. 4 and 5), or comprise genes in pathways of up and/or down regulated genes identified in accordance with the invention.

In embodiments of the invention, a gene is provided which is selected from the group consisting of the genes set forth in Table 2, which gene is an up-regulated biomarker of pre-term labor.

In embodiments of the invention, a gene is provided which is selected from the group consisting of the genes set forth in Table 3, which gene is a down-regulated biomarker of pre-term labor.

The invention also contemplates a sequence selected from the group consisting of the genes and sequences identified in Tables 2, 3, 4, 5, and/or 6, or SEQ ID Nos. 1 through 232, and combinations thereof, which if a molecular target for therapeutics of pre-term labor or for the discovery of therapeutics for pre-term labor.

The invention also contemplates protein marker sets that distinguish preterm labor and term labor, the protein marker sets comprising or consisting essentially of at least 5, 10, 15, 20, 25, 30, 35, 40, 45, or 50 of the proteins expressed by marker polynucleotides listed in Table 2, 3, 4, 5, and/or 6, or SEQ ID Nos. 1 through 232. In certain aspects the plurality of proteins consists of at least 50, 60, 70, 80, 90, 100, 110, 120, 130, 140, 150, 160, 170, 180, 190, 200, 210, 220, 230, 240, 250, 260, 270, 280, 290, or 300 of the proteins expressed by marker polynucleotides listed in Table 2, 3, 4, and/or 6, or SEQ ID Nos. 1 through 232. In an aspect the protein marker sets comprise or consist of protein clusters, or proteins in pathways comprising the markers.

The protein markers of the invention including but not limited to native-sequence polypeptides, isoforms, chimeric polypeptides, all homologs, fragments, and precursors of the markers, including modified forms of the polypeptides and derivatives are referred to herein as “Pre-term Labor Marker(s)” or “PLM Markers”. Polynucleotides encoding Pre-term Labor Markers or expressing PLM Markers are referred to herein as “Pre-term Labor Polynucleotide Marker(s)”, “polynucleotides encoding Pre-term Labor Marker(s)” or “PLM Polynucleotides”. The PLM Markers and PLM Polynucleotides are sometimes collectively referred to herein as “marker(s)”.

PLM polynucleotides associated with pre-term labor or onset of pre-term labor identified in accordance with a method of the invention, (including the markers listed in Table 2, 3, 4, 5, and/or 6, or SEQ ID Nos. 1 through 232), and polypeptides expressed from the PLM polynucleotides, have application in the determination of the status of pre-term labor, and in particular in the detection of pre-term labor or onset of pre-term labor. Thus, the markers can be used for diagnosis, monitoring (i.e. monitoring progression or therapeutic treatment), prognosis, treatment, or classification of pre-term labor, or as markers before or after therapy.

The levels of PLM polynucleotides or PLM Markers in a sample may be determined by as described herein and generally known in the art. The expression levels may be determined by isolating and determining the level of nucleic acid transcribed from each PLM Polynucleotide. Alternatively or additionally, the levels of PLM Markers translated from mRNA transcribed from a PLM polynucleotide may be determined.

In accordance with methods of the invention, susceptibility to pre-term labor can be assessed or characterized, for example by detecting or identifying the presence in the sample of (a) a PLM Marker or fragment thereof; (b) a metabolite which is produced directly or indirectly by a PLM Marker; (c) a transcribed polynucleotide or fragment thereof having at least a portion with which a PLM Polynucleotide is substantially identical; and/or (c) a transcribed polynucleotide or fragment thereof, wherein the polynucleotide hybridizes with a PLM Polynucleotide.

In an aspect, the invention provides a method for characterizing or classifying a sample as pre-term labor comprising detecting a difference in the expression of a first plurality of genes relative to a control, the first plurality of genes consisting of at least 5, 10, 15, 20, 25, 30, 35, 40, 45, or 50 of the genes corresponding to the markers listed in Table 2, 3, 4, 5, and/or 6, or SEQ ID Nos. 1 through 232. In particular aspects, the plurality of genes consists of at least 50, 60, 70, 80, 90, 100, 110, 120, 130, 140, 150, 160, 170, 180, 190, 200, 210, 220, 230, 240, 250, 260, 270, 280, 290, or 300 of the gene markers listed in Table 2, 3, 4, and/or 6, or SEQ ID Nos. 1 through 232. In another particular aspect, the control comprises polynucleotides derived from a pool of samples from individual term patients.

In an aspect, a method is provided characterizing susceptibility to pre-term labor by detecting PLM Markers or PLM Polynucleotides in a subject comprising:
(a) obtaining a sample from a subject;
(b) detecting or identifying in the sample PLM Markers or PLM Polynucleotides; and
(c) comparing the detected amount with an amount detected for a standard.

In an embodiment of the invention, a method is provided for detecting PLM Markers or PLM Polynucleotides in a subject or for diagnosing or monitoring in a subject a condition requiring regulation of labor comprising:
(a) obtaining a sample from a patient;
(b) detecting in the sample PLM Markers or PLM Polynucleotides; and
(c) comparing the detected amount with an amount detected for a standard.

The term “detect” or “detecting” includes assaying, imaging or otherwise establishing the presence or absence of the target markers or polynucleotides encoding the markers, subunits thereof, or combinations of reagent bound targets, and the like, or assaying for, imaging, ascertaining, establishing, or otherwise determining one or more factual characteristics of pre-term labor or similar conditions. The term encompasses diagnostic, prognostic, and monitoring applications for the PLM Markers and PLM Polynucleotides.

The invention also provides a method of assessing whether a patient has pre-term labor or a pre-disposition for pre-term labor comprising comparing:
(a) levels of PLM Markers or PLM Polynucleotides in a sample from the patient; and
(b) normal levels of PLM Markers or PLM Polynucleotides in samples of the same type obtained from control patients who delivered to term, wherein altered levels of the PLM Markers or PLM Polynucleotides relative to the corresponding normal levels of the markers or polynucleotides is an indication that the patient has pre-term labor or has a predisposition to pre-term labor.

In an embodiment of a method of the invention for assessing whether a patient has pre-term labor or a pre-disposition for pre-term labor, higher levels of PLM Markers or PLM Polynucleotides in a sample relative to the corresponding normal levels is an indication that the patient has pre-term labor or a pre-disposition for pre-term labor. In a particular embodiment the PLM Polynucleotides are the sequences listed on Table 2.

In another particular embodiment of a method of the invention for assessing whether a patient has pre-term labor or a pre-disposition for pre-term labor, lower levels of PLM Markers or PLM Polynucleotides in a sample relative to the corresponding normal levels is an indication that the patient has pre-term labor or a pre-disposition for pre-term labor. In a particular embodiment the PLM Polynucleotides are the sequences listed on Table 3.

In an embodiment of the invention, a method for screening or monitoring a subject for pre-term labor is provided comprising (a) obtaining a biological sample from a subject; (b) detecting the amount of PLM Markers or PLM Polynucleotides associated with pre-term labor in said sample; and (c) comparing said amount of PLM Markers or PLM Polynucleotides detected to a predetermined standard, where detection of a level of PLM Markers or PLM Polynucleotides that differs significantly from the standard indicates pre-term labor or onset of pre-term labor.

A significant difference between the levels of PLM Marker or PLM Polynucleotide levels in a patient and the normal levels is an indication that the patient has pre-term labor or a predisposition to pre-term labor.

In an embodiment the amount of PLM Marker(s) or PLM Polynucleotide(s) (e.g. see markers in Table 2) detected is greater than that of a standard and is indicative of pre-term labor. In another embodiment the amount of PLM Marker(s) or PLM Polynucleotide(s) (e.g. see markers in Table 3) detected is lower than that of a standard and is indicative of pre-term labor or onset of pre-term labor.

A method of diagnosing or monitoring pre-term labor or onset of pre-term labor in a subject is provided comprising obtaining a biological sample from the subject, identifying polynucleotides in the sample associated with pre-term labor to identify pre-term labor of a particular etiology, and providing an individualized therapeutic strategy based on the etiology of pre-term labor identified.

In one aspect the invention provides a method for determining pre-term labor development potential in a patient at risk for the development of pre-term labor comprising the steps of determining the concentration of one or more markers in Table 2, 3, 4, 5, and/or 6, or SEQ ID Nos. 1 through 232, in a sample (e.g. serum or plasma) from the patient, comparing the concentration of the markers to a cut-off concentration and determining pre-term development potential from the comparison, wherein concentrations of markers above the cut-off concentration are predictive of (e.g., correlate with) pre-term labor development in the patient.

In aspects of the methods of the invention, the methods are non-invasive for detecting pre-term labor, which in turn allow for diagnosis of a variety of conditions or diseases associated with such pre-term labor or conditions requiring regulation of labor.

In particular, the invention provides a non-invasive non-surgical method for detection, diagnosis, monitoring, or prediction of term or pre-term labor or onset of pre-term labor in a pregnant female comprising: obtaining a sample of blood, plasma, serum, urine or saliva or a tissue sample from the pregnant female; subjecting the sample to a procedure to detect PLM Marker(s) or PLM Polynucleotide(s) in the blood, plasma, serum, urine, saliva or tissue; detecting, diagnosing, and predicting term or pre-term labor by comparing the levels of PLM Marker(s) or PLM Polynucleotide(s) to the levels of PLM Marker(s) or PLM Polynucleotide(s) obtained from a pregnant non-laboring female.

In an embodiment, term or pre-term labor or onset of pre-term labor is detected, diagnosed, or predicted by determination of decreased levels of markers (e.g Table 3 markers) when compared to such levels obtained from the pregnant non-laboring female.

In another embodiment, term or pre-term labor or onset of pre-term labor is detected, diagnosed, or predicted by determination of increased levels of markers (e.g. Table 2 markers) when compared to such levels obtained from the pregnant non-laboring female.

The invention provides a method for monitoring the progression of pre-term labor in a patient the method comprising:
(a) detecting PLM Markers or PLM Polynucleotides in a sample from the patient at a first time point;
(b) repeating step (a) at a subsequent point in time; and
(c) comparing the levels detected in (a) and (b), and therefrom monitoring the progression of the pre-term labor.

The invention also provides a method for assessing the potential efficacy of a test agent for preventing, inhibiting, or reducing pre-term labor or onset of pre-term labor, and a method of selecting an agent for inhibiting pre-term labor.

The invention also contemplates a method of assessing the potential of a test compound to contribute to pre-term labor or onset of pre-term labor comprising:
(a) maintaining separate aliquots of tissue from a patient in the presence and absence of the test compound; and
(b) comparing the levels of PLM Markers or PLM Polynucleotides in each of the aliquots.

A significant difference between the levels of PLM Markers or PLM Polynucleotides in an aliquot maintained in the presence of (or exposed to) the test compound relative to the aliquot maintained in the absence of the test compound, indicates that the test compound potentially contributes to pre-term labor or onset of pre-term labor.

A method for determining the effect of an environmental factor on pre-term birth comprising comparing polynucleotides associated with pre-term labor or onset of pre-term labor in the presence and absence of the environmental factor.

The invention further relates to a method of assessing the efficacy of a therapy for preventing, inhibiting, or reducing pre-term labor or onset of pre-term labor in a patient. A method of the invention comprises comparing: (a) levels of PLM Markers or PLM Polynucleotides in a sample from the patient obtained from the patient prior to providing at least a portion of a therapy to the patient; and (b) levels of PLM Markers or PLM Polynucleotides in a second sample obtained from the patient following therapy.

A significant difference between the levels of PLM Markers or PLM Polynucleotides in the second sample relative to the first sample is an indication that the therapy is efficacious for inhibiting pre-term labor or onset of pre-term labor.

In an embodiment, the method is used to assess the efficacy of a therapy for inhibiting pre-term labor or onset of pre-term labor, where lower levels of PLM Markers or PLM Polynucleotides (e.g. Table 3 markers) relative to the first sample, is an indication that the therapy is efficacious for inhibiting the disease.

In an embodiment, the method is used to assess the efficacy of a therapy for inhibiting pre-term labor or onset of pre-term labor, where higher levels of PLM Markers or PLM Polynucleotides (e.g. Table 2 markers) relative to the first sample, is an indication that the therapy is efficacious for inhibiting pre-term labor or onset of pre-term labor.

The “therapy” may be any therapy for treating pre-term labor or onset of pre-term labor in particular, including but not limited to therapeutics, and procedures and interventions such as antenatal glucocorticoids and tocolysis. A method of the invention can be used to evaluate a patient before, during, and after therapy.

Certain methods of the invention employ one or more polynucleotides capable of hybridizing to one or more PLM Polynucleotides. Thus, methods for monitoring pre-term labor or onset of pre-term labor are contemplated comprising detecting PLM Polynucleotide markers associated with pre-term labor.

Thus, the present invention relates to a method for diagnosing and monitoring pre-term labor or onset of pre-term labor in a sample from a subject comprising isolating polynucleotides, in particular mRNA, from the sample; and detecting PLM Polynucleotides in the sample. The presence of different levels of PLM Polynucleotides in the sample compared to a standard or control may be indicative of pre-term labor, stage of pre-term labor, onset of pre-term labor, and/or a positive prognosis.

In an embodiment of the invention, PLM Polynucleotide positive samples (e.g. higher levels of the PLM Polynucleotides compared to a normal control) are a negative diagnostic indicator. Positive tissue can be indicative of pre-term labor, advanced pre-term labor, onset of pre-term labor, or a poor prognosis.

In another embodiment of the invention, PLM Polynucleotide negative samples (e.g. lower levels of the PLM Polynucleotides compared to a normal control) are a negative diagnostic indicator. Negative tissues can be indicative of pre-term labor, advanced pre-term labor, onset of pre-term labor, or poor prognosis.

The invention provides methods for determining the presence or absence of pre-term labor in a subject comprising detecting in the sample levels of polynucleotides that hybridize to one or more PLM Polynucleotides, comparing the levels with a predetermined standard or cut-off value, and therefrom determining the presence or absence of pre-term labor in the subject. In an embodiment, the invention provides methods for determining the presence or absence of pre-term labor in a subject comprising (a) contacting a sample obtained from the subject with oligonucleotides that hybridize to one or more PLM Polynucleotides; and (b) detecting in the sample a level of polynucleotides that hybridize to the PLM Polynucleotides relative to a predetermined cut-off value, and therefrom determining the presence or absence of pre-term labor in the subject.

Within certain embodiments, the amount of polynucleotides that are mRNA are detected via polymerase chain reaction using, for example, oligonucleotide primers that hybridize to one or more PLM Polynucleotides, or complements of such polynucleotides. Within other embodiments, the amount of mRNA is detected using a hybridization technique, employing oligonucleotide probes that hybridize to one or more PLM Polynucleotides, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more PLM Polynucleotides in the sample. For mRNA the analyzing step may be accomplished using Northern Blot analysis to detect the presence of PLM Polynucleotides. The analysis step may be further accomplished by quantitatively detecting the presence of PLM Polynucleotides in the amplification product, and comparing the quantity of marker detected against a panel of expected values for the known presence or absence of the markers in normal tissue derived using similar primers.

The invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more PLM Polynucleotides to produce amplification products; (d) analyzing the amplification products to detect an amount of mRNA encoding the PLM Markers; and (e) comparing the amount of mRNA to an amount detected against a panel of expected values for normal tissue derived using similar nucleic acid primers.

In particular aspects of the invention, the methods described herein utilize the PLM Polynucleotides placed on a micro-array so that the expression status of each of the markers is assessed simultaneously.

In an embodiment, the invention provides a pre-term labour micro-array comprising a defined set of genes whose expression is significantly altered by pre-term labour. The invention further relates to the use of the micro-array as a prognostic tool to predict pre-term delivery. In an embodiment, the pre-term labour micro-array discriminates between pre-term labor resulting from different etiologies.

In an embodiment, the invention provides for oligonucleotide arrays comprising marker sets described herein. The microarrays provided by the present invention may comprise probes to markers able to distinguish pre-term labor. In particular, the invention provides oligonucleotide arrays comprising probes to a subset or subsets of at least 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, or 300 gene markers (e.g. PLM Polynucleotides) up to a full set of markers which distinguish pre-term labor patients or samples.

The level of expression of the PLM Polynucleotides may be assessed by determining the levels of specific proteins expressed from the polynculeotides (i.e. the levels of the PLM Markers). Certain methods of the invention employ binding agents (e.g. antibodies) that specifically recognize PLM Markers.

In an embodiment, the invention provides methods for determining the presence or absence of pre-term labor or onset of pre-term labor, in a patient, comprising the steps of (a) contacting a biological sample obtained from a patient with one or more binding agent that specifically binds to one or more PLM Markers associated with pre-term labor; and (b) detecting in the sample an amount of marker that binds to the binding agent, relative to a predetermined standard or cut-off value, and therefrom determining the presence or absence of pre-term labor in the patient.

In another embodiment, the invention relates to a method for diagnosing and monitoring pre-term labor in a subject by quantitating one or more PLM Markers associated with pre-term labor in a biological sample from the subject comprising (a) reacting the biological sample with one or more binding agent specific for the PLM Markers (e.g. an antibody) that are directly or indirectly labelled with a detectable substance; and (b) detecting the detectable substance.

In another aspect the invention provides a method for using an antibody to detect expression of one or more PLM Marker in a sample, the method comprising: (a) combining antibodies specific for one or more PLM Marker with a sample under conditions which allow the formation of antibody:marker complexes; and (b) detecting complex formation, wherein complex formation indicates expression of the marker in the sample. Expression may be compared with standards and is diagnostic of pre-term labor.

PLM Markers levels can be determined by constructing an antibody microarray in which binding sites comprise immobilized, preferably monoclonal, antibodies specific to a substantial fraction of marker-derived proteins of interest.

The invention also relates to kits for carrying out the methods of the invention. In an embodiment, the kit is for assessing whether a patient is afflicted with a pre-term labor and it comprises reagents for assessing one or more PLM Markers or PLM Polynucleotides. In another embodiment, the invention provides diagnostic tools, and kits for detecting, diagnosing, and predicting the presence or impending onset of premature or pre-term labor by monitoring levels of PLM Markers or a PLM Polynucleotides.

The invention further provides kits comprising the marker sets described herein. In an aspect the kit contains a micro-array ready for hybridization to target PLM Polynucleotides, plus software for the data analyses.

The invention also provides a diagnostic composition comprising a PLM Marker or a PLM Polynucleotide. A composition is also provided comprising a probe that specifically hybridizes to PLM Polynucleotides, or a fragment thereof, or an antibody specific for PLM Markers or a fragment thereof. In another aspect, a composition is provided comprising one or more PLM Polynucleotide specific primer pairs capable of amplifying the polynucleotides using polymerase chain reaction methodologies. The probes, primers or antibodies can be labeled with a detectable substance.

Still further the invention relates to therapeutic applications for pre-term labor employing PLM Markers and PLM Polynucleotides, and/or binding agents for the markers.

In an aspect, the invention relates to pharmaceutical compositions comprising PLM Markers or parts thereof associated with pre-term labor, or binding agents (e.g antibodies) specific for PLM Markers associated with pre-term labor, and a pharmaceutically acceptable carrier, excipient, or diluent.

The invention provides a method of treating or preventing pre-term labor or onset of pre-term labor in a subject afflicted with or at risk of developing pre-term labor comprising administering to the subject an effective amount of an agonist of a down-regulated PLM Marker or PLM Polynucleotide. The term agonist is used in its broadest sense. Agonist can include any agent that results in activation, enhancement or alteration of the presence of a down-regulated PLM Marker or PLM Polynucleotide.

The invention provides a method of treating or preventing pre-term labor or onset of pre-term labor in a subject having or at risk of developing pre-term labor comprising administering to the subject an effective amount of an antagonist of an up-regulated PLM Marker or PLM Polynucleotide. The term antagonist or antagonizing is used in its broadest sense. Antagonism can include any mechanism or treatment that results in inhibition, inactivation, blocking or reduction or alteration of the presence of an up-regulated PLM Marker or PLM Polynucleotide. Examples of antagonists are antibodies specific for PLM Markers, binding agents for PLM Markers, and inhibitors of PLM Polynucleotides (e.g. antisense).

A method for treating or preventing pre-term labor or onset of pre-term labor in a subject is provided comprising administering to a subject in need thereof antibodies specific for PLM Markers. In an aspect the invention provides a method of treating a subject afflicted with or at risk of developing pre-term labor comprising inhibiting expression of one or more PLM Marker or PLM Polynucleotide.

In another aspect, the invention provides antibodies specific for PLM Markers associated with pre-term labor that can be used to inhibit PLM Marker or PLM Polynucleotide expression.

The invention contemplates a method of using antagonists or agonists of PLM Markers or PLM Polynucleotides or parts thereof in the preparation or manufacture of a medicament for the prevention or treatment of pre-term labor.

In an aspect the invention contemplates a method of using PLM Markers or parts thereof, antibodies specific for PLM Markers, or inhibitor of PLM Polynucleotides (e.g. antisense) in the preparation or manufacture of a medicament for the prevention or treatment of pre-term labor or onset of pre-term labor.

The invention also provides a method for stimulating or enhancing in a subject production of antibodies directed against one or more up-regulated PLM Marker. The method comprises administering to the subject one or more up-regulated PLM Marker, peptides derived therefrom, or chemically produced (synthetic) peptides, or any combination of these molecules of the invention in a dose effective for stimulating or enhancing production of the antibodies.

The invention contemplates the methods, compositions, and kits described herein using additional markers associated with pre-term labor (e.g. fibronectin). The methods described herein may be modified by including reagents to detect the additional markers, or polynucleotides for the markers.

In embodiments of the invention the methods, compositions and kits use one or more of the markers listed in Table 2, 3, 4, 5, and/or 6, or SEQ ID Nos. 1 through 232. In another embodiment, they use a panel of markers selected from the markers listed in Table 2, 3, 4, 5, and/or 6, or SEQ ID Nos. 1 through 232, in particular a panel comprising two or more of the markers in Table 4, 5, or 6, or SEQ ID Nos. 1 through 232.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1. The scatter plot presents the relative gene expressions of 18879 EST's comparing the T-PTL that progressed to delivery (delivered) group to the T-PTL whose pregnancies continued to term (undelivered) group. In the women who progressed to delivery, there were 266 EST's (labelled red) whose expression was increased more than 2 fold [range 2-9 fold] and 561 EST's (labelled blue) whose expression was decreased more than 2 fold [range 2-25 fold] as compared to those who continued to term gestations.

FIG. 2. The 5 most differentially expressed EST's are presented in this figure. WDR5B is a gene that codes for a protein involved in protein-protein interactions. KCNMA1 is the gene for the MaxiK channels, which are large conductance, voltage and calcium sensitive potassium channels that are fundamental to the control of smooth muscle tone and neuronal excitability.

FIG. 3. Gene expression of 18879 genes sorted by difference between 2 groups. In this Figure red indicates EST's with increased expression, green indicates EST's with decreased expression and black indicates EST's where there is no difference in expression between the two study groups. Even without cluster analysis this figure clearly indicates the magnitude of the differences in gene expression in leukocytes between the two experimental groups.

FIG. 4. Cluster analysis of 216 predictive EST's using complete linkage of Euclidean distance. The dendogram illustrates the division into delivered vs. undelivered women at the first division in the dendogram. The cluster analysis of the genes in red suggests that there may be subsets of EST's that may have the potential to identify specific types of pre-term labour.

FIG. 5. Cluster analysis of 52 predictive EST's using complete linkage of Euclidean distance. The division into delivered vs. undelivered occurs at the first division of the dendogram

FIG. 6. Cluster analysis of 2 genes with the maximum difference between the two groups allows prediction of timing of delivery, again at the first division of the dendogram.

FIG. 7. Pathway analysis of the common regulators of the genes with maximal differences between the two experimental groups. Genes that are members of the dataset are coloured red whereas regulators which have been added to the pathway are coloured blue.

FIG. 8. Pathway analysis of the genes whose expression is increased greater than two fold in the women with T-PTL who progress to delivery.

FIG. 9. Pathway analysis of the genes whose expression is decreased greater than two fold in the women with T-PTL who progress to delivery.

FIG. 10. Combined pathway of up and down regulated gene expression in women with T-PTL who progress to delivery. Genes coloured red are from the down regulated set and those coloured green are from the up-regulated set.

### DETAILED DESCRIPTION OF THE INVENTION

Methods are provided for detecting the presence of pre-term labor in a sample, the absence of pre-term labor, stage of pre-term labor, and other characteristics of pre-term labor that are relevant to prevention, diagnosis, monitoring, characterization, and therapy of pre-term labor in a patient. Methods are also provided for assessing the efficacy of one or more test agents for preventing, inhibiting, or reducing pre-term labor, assessing the efficacy of a therapy for pre-term labor, monitoring the progression of pre-term labor, selecting an agent or therapy for pre-term labor, treating a patient afflicted with pre-term labor, preventing, inhibiting, or reducing pre-term labor in a patient, and assessing the potential of a test compound to cause pre-term labor.

Glossary

“Pre-term labor”, refers to the premature onset of labor resulting in expulsion from the uterus of a viable infant before the normal end of gestation (i.e. pre-term birth or delivery), or more particularly, onset of labor with effacement and dilation of the cervix before the 37th week of gestation. It may or may not be associated with vaginal bleeding or rupture of membranes. Pre-term labor may be related to factors including without limitation infection (eg, bacterial vaginosis [BV], sexually transmitted diseases [STDs], urinary tract infections, chorioamnionitis), uterine distention (eg, multiple gestation, polyhydramnios), uterine distortion (eg, müllerian duct abnormalities, fibroid uterus), compromised structural support of the cervix (eg, incompetent cervix, previous cone biopsy or loop electrosurgical excision procedure [LEEP]), abruptio placentae, uteroplacental insufficiency (eg, hypertension, insulin-dependent diabetes, drug abuse, smoking, alcohol consumption), stress either indirectly by associated risk behaviors or by direct mechanisms including fetal stress.

“Threatened pre-term labor” refers to premature onset of labor before the 37^{th} week of gestation followed by a continuation of pregnancy to term (“term labor”) or pre-term labor. Symptoms of threatened pre-term labor include without limitation regular uterine contractions, cervical dilation 0-4 cm, and/or intact fetal membranes.

“Micro-array” and “array,” refer to nucleic acid or nucleotide arrays or protein or peptide arrays that can be used to detect biomolecules associated with pre-term labor, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)].

The terms “sample”, “biological sample”, and the like mean a material known or suspected of expressing or containing one or more PLM Polynucleotides and/or one or more PLM Markers. A test sample can be used directly as obtained from the source or following a pretreatment to modify the character of the sample. A sample can be derived from any biological source, such as tissues, extracts, or cell cultures, including cells, cell lysates, and physiological fluids, such as, for example, whole blood, plasma, serum, saliva, ocular lens fluid, cerebral spinal fluid, sputum, sweat, urine, milk, ascites fluid, synovial fluid, peritoneal fluid, and the like.

The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like.

In embodiments of the invention the sample is blood, in particular blood cells, particularly maternal peripheral blood cells, more particularly mononuclear leukocytes.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. No. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Moelcular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labeled differentially from polynucleotides of a standard. The standard can comprise target polynucleotides from normal individuals (i.e., those not afflicted with or pre-disposed to pre-term labor), in particular pooled from samples from normal individuals. The target polynucleotides can be derived from the same individual, but taken at different time points, and thus indicate the efficacy of a treatment by a change in expression of the markers, or lack thereof, during and after the course of treatment.

The terms “subject”, “individual” or “patient” refer to a warm-blooded animal such as a mammal. In particular, the terms refer to a human. A subject, individual or patient may be afflicted with or suspected of having or being pre-disposed to pre-term labor. The present invention may be particularly useful for determining pre-term labor development potential in at-risk patients suffering from particular pre-term labor predisposing conditions. Pre-term labor predisposing conditions include without limitation a previous history of preterm delivery, previous history of a second-trimester abortion, uterine factors such as uterine volume increase, uterine anomalies, trauma and infection.

The term “PLM Marker” or “Pre-term labor Markers” includes a marker associated with pre-term labor. The term includes native-sequence polypeptides isoforms, chimeric polypeptides, complexes, all homologs, fragments, precursors, and modified forms and derivatives of the markers. The term includes a marker associated with pre-term labor identified using a method of the invention, in particular a marker expressed by a polynucleotide listed in Table 2, 3, 4, 5 and/or 6, or SEQ ID Nos. 1 through 232.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide. Particular polypeptide variants have at least 70-80%, 85%, 90%, 95% amino acid sequence identity to the sequences of the proteins expressed by the polynucleotides identified in Table 2, 3, 4, 5, and/or 6, or SEQ ID Nos. 1 through 232. Such variants include, for instance, polypeptides wherein one or more amino acid residues are added to, or deleted from, the N- or C-terminus of the full-length or mature sequences of the polypeptide, including variants from other species, but excludes a native-sequence polypeptide.

The invention also includes polypeptides that are substantially identical to the sequences of a PLM Marker, in particular a pre-term labor marker, more particularly a marker expressed by a polynucleotide listed in Table 2, 3, 4, or 5, or SEQ ID Nos. 1 through 232 (e.g. at least about 45%, preferably 50%, 55%, 60%, 65%, 70%, 75%, 80%, 85%, 90%, 95%, 97%, 98%, or 99% sequence identity), and in particular polypeptides that retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine or rat polypeptide.

PLM Markers include chimeric or fusion proteins. A “chimeric protein” or “fusion protein” comprises all or part (preferably biologically active) of a PLM Marker operably linked to a heterologous polypeptide (i.e., a polypeptide other than a PLM Marker). Within the fusion protein, the term “operably linked” is intended to indicate that a PLM Marker and the heterologous polypeptide are fused in-frame to each other. The heterologous polypeptide can be fused to the N-terminus or C-terminus of a PLM Marker. A useful fusion protein is a GST fusion protein in which a PLM Marker is fused to the C-terminus of GST sequences. Another example of a fusion protein is an immunoglobulin fusion protein in which all or part of a PLM Marker is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including but not limited to glycosylated, phosphorylated, acetylated, methylated or lapidated forms of the polypeptides.

PLM Markers may be prepared by recombinant or synthetic methods, or isolated from a variety of sources, or by any combination of these and similar techniques.

“Pre-term Labor Polynucleotides”, “PLM Polynucleotide(s)”, or “polynucleotides encoding pre-term labor markers” refers to polynucleotides associated with pre-term labor and/or encoding PLM Markers including native-sequence polypeptides, polypeptide variants including a portion of a polypeptide, an isoform, precursor, complex, a chimeric polypeptide, or modified forms and derivatives of the polypeptides. A PLM Polynucleotides can be a polynucleotide listed in Table 2, 3, 4, 5, and/or 6, or SEQ ID Nos. 1 through 232.

In a particular embodiment, a polynucleotide of the invention is WDR5B which includes the sequences of WDR5B shown as SEQ ID NO. 1 or Accession No. R12819, or a fragment thereof.

In another particular embodiment, a polynucleotide of the invention is KCNMA1 which includes the sequences of KCNMA1 shown as SEQ ID NO. 2 or Accession No. R11947, or a fragment thereof.

In another particular embodiment, a polynucleotide of the invention is PTGS2D which includes the sequences of PTGS2 shown as SEQ ID NO. 30 or Accession No. R80322, or a fragment thereof.

In another particular embodiment, a polynucleotide of the invention comprises a sequence of Table 4 or SEQ ID Nos. 1 to 39, or a fragment thereof.

In another particular embodiment, a polynucleotide of the invention comprises a sequence of Table 5 or SEQ ID Nos. 1, 2, 3, 4, and/or 5, or a fragment thereof.

In another particular embodiment, a polynucleotide of the invention comprises a sequence of Table 6 or SEQ ID Nos. 40 to 232, or a fragment thereof.

PLM Polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

PLM Polynucleotides also include sequences that differ from a native sequence due to degeneracy in the genetic code. As one example, DNA sequence polymorphisms within the nucleotide sequence of a PLM Polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides also include nucleic acids that hybridize under stringent conditions, preferably high stringency conditions to a PLM Polynucleotide. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

PLM Polynucleotides also include truncated nucleic acids or nucleic acid fragments and variant forms of the nucleic acids that arise by alternative splicing of an mRNA corresponding to a DNA.

PLM Polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense polynucleotide markers).

“Statistically different levels”, “significantly altered”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 3, 4, 5, or 6 times higher or lower than the control or standard.

“Binding agent” refers to a substance such as a polypeptide or antibody that specifically binds to one or more PLM Marker. A substance “specifically binds” to one or more PLM Marker if is reacts at a detectable level with one or more PLM Marker, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more PLM Marker sequence, a peptide variant thereof, or a non-peptide mimetic of such a sequence.

An aptamer includes a DNA or RNA molecule that binds to nucleic acids and proteins. An aptamer that binds to a protein (or binding domain) or a PLM Polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant PLM Markers may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for a PLM Marker may also be obtained from scientific or commercial sources. In an embodiment of the invention, antibodies are reactive against a PLM Marker if they bind with a Kₐ of greater than or equal to 10⁻⁷ M.

Markers

The invention provides a set of markers correlated with pre-term labor by clustering analysis. A subset of these markers identified as useful for detection, diagnosis, prevention and therapy of pre-term labor is listed in Table 2, 3, 4, 5, and/or 6, or SEQ ID Nos. 1 through 232. The invention also provides a method of using these markers to distinguish threatened pre-term labor that progresses to delivery from pregnancies that continue to term.

The invention provides gene marker sets that distinguish preterm labor and term labor and uses of such markers. In an aspect, the invention provides a method for classifying a sample as pre-term labor comprising detecting a difference in the expression of a first plurality of genes relative to a control, the first plurality of genes consisting of at least 5, 10, 15, 20, 25, 30, 35, 40, 45, or 50 of the genes corresponding to the markers listed in Table 2, 3, 4, 5, and/or 6, or SEQ ID Nos. 1 through 232. In specific aspects, the plurality of genes consists of at least 50, 100, 200, or 300 of the gene markers listed in Table 2, 3, 4, 5, and/or 6, or SEQ ID Nos. 1 through 232. In another specific aspect, the control comprises nucleic acids derived from a pool of samples from individual term patients.

Any of the markers provided herein may be used alone or with other markers of pre-term labor, or with markers for other phenotypes or conditions.

Identification of PLM Markers

As mentioned herein, the present invention provides sets of markers for detecting, diagnosing and predicting pre-term labor or onset of pre-term labor in patient samples. Generally, marker sets were identified by determining which human markers had expression patterns that correlated with pre-term labor.

Thus, the invention relates to a method of characterizing a sample, in particular a peripheral blood leukocyte sample, by detecting or quantitating in the sample one or more polynucleotides extracted from the sample that are characteristic of pre-term labor the method comprising assaying for differential expression of polynucleotides in the sample. Differential expression of the polynucleotides can be determined by micro-array analysis or by amplification of the extracted polynucleotides.

In an embodiment, a method for identifying sets or markers is provided comprising extracting and labelling target polynucleotides, and comparing the expression of all markers (genes) in a sample to the expression of all markers in a standard or control. The sample may comprise a single sample, or a pool of samples; the samples in the pool may come from different individuals. In one embodiment, the standard or control comprises target polynucleotide molecules derived from a sample from a normal individual (i.e., an individual not afflicted or pre-disposed to pre-term labor). In a particular embodiment, the standard or control is a pool of target polynucleotides derived from collected samples from a number of normal individuals.

Comparison of the patient sample and control may be accomplished by any means known in the art. By way of example, expression levels of various markers can be assessed by separation of target polynucleotides (e.g., RNA or cDNA) derived from the markers in agarose or polyacrylamide gels, followed by hybridization with marker-specific oligonucleotide probes. In the alternative, the comparison may be accomplished by the labeling of target polynucleotides followed by separation on a sequencing gel. The patient and control or standard polynucleotides can be in adjacent lanes. Expression levels can be compared visually or using a densitometer. In a particular embodiment, the expression of all markers is assessed simultaneously by hybridization to an oligonucleotide microarray. In each approach, markers meeting certain criteria are identified as associated with pre-term labor.

Markers can be selected based upon a significant difference of expression (up- or down-regulation) in a sample as compared to a standard or control. Markers can also be selected by calculation of the statistical significance (i.e., the p-value) of the correlation between the expression of the marker and pre-term labor. Both selection criteria are generally used. In an aspect of the invention, markers associated with pre-term labor are selected where the markers show more than two-fold change (increase or decrease) in expression as compared to a standard, and/or the p-value for the correlation between pre-term labor and the change in marker expression is no more than 0.01 (i.e., is statistically significant).

The expression of the identified pre-term markers can be used to identify markers that can differentiate pre-term labor into clinical types.

In particular aspects of the invention a method is provided for identifying markers associated with pre-term labor comprising:
(a) obtaining peripheral blood leukocytes from a subject;
(b) extracting polynucleotides from the peripheral blood leukocytes and producing a microarray profile of the polynucleotides; and
(c) comparing the profile with a profile for peripheral blood leukocytes from a normal individual to identify polynucleotides associated with pre-term labor.

The profile of nucleic acids can be produced by a microarray or by amplification of the nucleic acids (e.g. using PCR).

In an aspect the invention provides a method of characterizing a sample (e.g peripheral blood leukocytes) by detecting or quantitating in the sample one or more polynucleotides extracted from the sample that are characteristic of pre-term labor the method comprising assaying for differential expression of polynucleotides in the sample by microarray of polynucleotides extracted from the sample.

The preparation, use, and analysis of microarrays are described herein and are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

The invention also relates to a method of characterizing a sample in particular peripheral blood leukocytes by detecting or quantitating in the sample one or more polypeptides extracted from the sample that are characteristic of pre-term labor the method comprising assaying for differential expression of polypeptides in the sample. Differential expression of polypeptides can be assayed by mass spectroscopy or an antibody microarray of polypeptides extracted from the sample.

The invention relates to a method for identifying PLM Markers associated with pre-term labor comprising:
(a) obtaining a sample of peripheral blood leukocytes from a subject;
(b) extracting polypeptides from the peripheral blood leukocytes and producing a profile of the polypeptides by subjecting the polypeptides to mass spectrometry; and
(c) comparing the profile with a profile for normal peripheral blood leukocytes or for a known stage or type of pre-term labor to identify polypeptides associated with pre-term labor.

Polypeptides may be extracted from the samples in a manner known in the art. For example, polypeptides may be extracted by first digesting or disrupting cell membranes by standard methods such as detergents or homogenization in an isotonic sucrose solution, followed by ultra-centrifugation or other standard techniques.

The separated polypeptides may be digested into peptides, in particular using proteolytic enzymes such as trypsin, pepsin, subtilisin, and proteinase. For example, polypeptides may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

Mass spectrometers that may be used to analyze the peptides or polypeptides include a Matrix-Assisted Laser Desorptioon/Ioniation Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

Detection Methods

A variety of methods can be employed for the detection, diagnosis, monitoring, and prognosis of pre-term labor, onset of pre-term labor, or status of pre-term labor involving one or more PLM Markers and/or PLM Polynucleotides, and for the identification of subjects with a predisposition to pre-term labor. Such methods may, for example, utilize PLM Polynucleotides, and fragments thereof, and binding agents (e.g. antibodies) against one or more PLM Markers, including peptide fragments. In particular, the polynucleotides and antibodies may be used, for example, for (1) the detection of the presence of PLM Polynucleotide mutations, or the detection of either an over- or under-expression of PLM Polynucleotide mRNA relative to a non-pre-term state, or the qualitative or quantitative detection of alternatively spliced forms of PLM Polynucleotide transcripts which may correlate with certain conditions or susceptibility toward pre-term labor; and (2) the detection of either an over- or an under-abundance of one or more PLM Markers relative to a non-pre-term labor state or a different stage or type of injury or the presence of a modified (e.g., less than full length) PLM Marker which correlates with a pre-term labor state or a progression toward pre-term labor, or a particular type or stage of pre-term labor.

If the gene(s) represent surface antigens or secreted peptides, antibodies can be raised and standard ELISA's developed. In addition, novel automated RNA extraction can be utilized, followed by multiplex, real time RT-PCR. For example, the MagNA Pure LC & LightCycler system from Roche Diagnostic is capable of accurately quantifying RNA expression in cells within 90 minutes.

The invention contemplates a method for detecting or monitoring the stage or type of pre-term labor or onset of pre-term labor, comprising producing a profile of levels of one or more PLM Marker and/or PLM Polynucleotides, and optionally other markers associated with pre-term labor in a sample from a patient, and comparing the profile with a reference to identify a profile for the patient indicative of the stage or type of pre-term labor.

The methods described herein may be used to evaluate the probability of the presence of pre-term labor or onset of pre-term labor, for example, in a sample freshly removed from a host. Such methods can be used to detect pre-term labor and help in the diagnosis and prognosis of pre-term labor. The methods can be used to detect the potential for pre-term labor and to monitor pre-term labor or a therapy.

The invention also contemplates a method for detecting pre-term labor or onset of pre-term labor comprising producing a profile of levels of one or more PLM Marker and/or PLM Polynucleotides, and other markers associated with pre-term labor in a sample (e.g. cells) from a patient, and comparing the profile with a reference to identify a profile for the patient indicative of pre-term labor.

The methods described herein can be adapted for diagnosing and monitoring pre-term labor by detecting one or more PLM Markers or PLM Polynucleotides in biological samples from a subject. These applications require that the amount of PLM Markers or PLM Polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of pre-term labor, may be established by prospective and/or retrospective statistical studies. Healthy subjects who have no clinically evident pre-term labor or abnormalities may be selected for statistical studies. Diagnosis may be made by a finding of statistically different levels of detected PLM Markers associated with pre-term labor or PLM Polynucleotides, compared to a control sample or previous levels quantitated for the same subject.

The methods described herein may also use multiple markers for pre-term labor. Therefore, the invention contemplates a method for analyzing a biological sample for the presence of one or more PLM Markers and PLM Polynucleotides, and other markers that are specific indicators of pre-term labor. The methods described herein may be modified by including reagents to detect the additional markers.

Nucleic Acid Methods/Assays

As noted herein pre-term labor or stage or type of same may be detected based on the level of PLM Polynucleotides in a sample. Techniques for detecting polynucleotides such as polymerase chain reaction (PCR) and hybridization assays are well known in the art.

Probes may be used in hybridization techniques to detect polynucleotide markers. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a PLM Polynucleotide, preferably they comprise 10-30, 10-40, 15-40, 20-50, 40-80, 50-150, or 80-120 nucleotides.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect PLM Polynucleotides in human samples, e.g. peripheral blood leukocytes. The nucleotide probes may also be useful in the diagnosis of pre-term labor involving one or more PLM Polynucleotides; in monitoring the progression of pre-term labor; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA is determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of PLM Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a PLM Polynucleotide(s) derived from a sample, wherein at least one of the oligonucleotide primers is specific for (i.e. hybridizes to) a PLM Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a PLM Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of PLM Polynucleotide expression. For example, RNA may be isolated from a cell type or tissue known to express a PLM Polynucleotide and tested utilizing the hybridization (e.g. standard Northern analyses) or PCR techniques referred to herein. The primers and probes may be used in the above-described methods in situ i.e. directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a PLM Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected pre-term labor and an individual who is not predisposed to pre-term labor. The reaction may be performed on several dilutions of cDNA spanning at least two orders of magnitude. A significant difference in expression in several dilutions of the subject sample as compared to the same dilutions of the normal sample may be considered positive for the presence of pre-term labor.

In an embodiment, the invention provides methods for determining the presence or absence of a pre-term labor in a subject comprising (a) contacting a sample obtained from the subject with oligonucleotides that hybridize to one or more PLM Polynucleotides; and (b) detecting in the sample a level of nucleic acids that hybridize to the polynucleotides relative to a predetermined cut-off value, and therefrom determining the presence or absence of pre-term labor in the subject.

The invention provides a method wherein an PLM Polynucleotide which is mRNA is detected by (a) isolating mRNA from a sample and combining the mRNA with reagents to convert it to cDNA; (b) treating the converted cDNA with amplification reaction reagents and nucleic acid primers that hybridize to one or more PLM Polynucleotides, to produce amplification products; (d) analyzing the amplification products to detect amounts of mRNA encoding PLM Polynucleotides; and (e) comparing the amount of mRNA to an amount detected against a panel of expected values for normal tissue derived using similar nucleic acid primers.

PLM Marker-positive samples or alternatively higher levels in patients compared to a control (e.g. normal tissue) may be indicative of pre-term labor or advanced pre-term labor, and/or that the patient is not responsive to or tolerant of a therapy. Alternatively, negative samples or lower levels compared to a control (e.g. normal samples or negative samples) may also be indicative of pre-term labor or advanced pre-term labor.

In another embodiment, the invention provides methods for determining the presence or absence of pre-term labor in a subject comprising (a) contacting a sample obtained from the subject with oligonucleotides that hybridize to one or more PLM Polynucleotides; and (b) detecting in the sample levels of polynucleotides that hybridize to the PLM Polynucleotides relative to a predetermined cut-off value, and therefrom determining the presence or absence of pre-term labor in the subject. In an embodiment, the PLM Polynucleotides encode one or more polynucleotides listed in Table 2, 3, 4, 5, and/or 6, or SEQ ID Nos. 1 through 232.

In a particular aspect, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a PLM Polynucleotide, to produce amplification products; (d) analyzing the amplification products to detect an amount of PLM Polynucleotide mRNA; and (e) comparing the amount of mRNA to an amount detected against a panel of expected values for normal subjects derived using similar nucleic acid primers.

Marker-positive samples or alternatively higher levels, in particular significantly higher levels of PLM Polynucleotides listed in Table 2, in particular KCNMA1 or WDR5B, in patients compared to a control (e.g. normal) are indicative of pre-term labor.

In another particular aspect, the invention provides a method wherein PLM Polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a PLM Polynucleotide, to produce amplification products; (d) analyzing the amplification products to detect an amount of PLM Polynucleotide mRNA; and (e) comparing the amount of mRNA to an amount detected against a panel of expected values for normal subjects derived using similar nucleic acid primers.

Marker-positive samples or alternatively lower levels, in particular significantly lower levels of PLM Polynucleotides listed in Table 3 in patients compared to a control (e.g. normal) are indicative of pre-term labor.

Oligonucleotides or longer fragments derived from PLM Polynucleotides may be used as targets in a micro-array as described herein. The micro-array can be used to simultaneously monitor the expression levels of large numbers of genes. The micro-array can also be used to identify genetic variants, mutations, and polymorphisms. The information from the micro-array may be used to determine gene function, to understand the genetic basis of pre-term labor, to diagnose pre-term labor, and to develop and monitor the activities of therapeutic agents.

Thus, the invention also includes an array comprising one or more PLM Polynucleotidess (in particular the markers listed in Table 2, 3, 4, 5, and/or 6, or SEQ ID Nos. 1 through 232), and optionally other markers. The array can be used to assay expression of PLM Polynucleotides in the array. The invention allows the quantitation of expression of one or more PLM Polynucleotides.

Micro-arrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Micro-arrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous (e.g. gel), or non-porous. For example, the probes can be attached to a nitrocellulose or nylon membrane or filter covalently at either the 3′ or the 5′ end of the polynucleotide probe. The solid support may be a glass or plastic surface. In an aspect of the invention hybridization levels are measured to micro-arrays of probes consisting of a solid support on the surface of which are immobilized a population of polynucleotides.

In accordance with embodiments of the invention, a micro-array is provided comprising a support or surface with an ordered array of hybridization sites or “probes” each representing one of the markers described herein. The micro-arrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Micro-arrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the micro-array is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the PLM Markers and PLM Polynucleotides are present on the array. In a preferred embodiment, the array comprises at least 5, 10, 15, 20, 25, 30, 40, 50, 60, 70, 80, 90, 100, 125, 150, 175, 200, 225, 250, 275, or 300 of the PLM Polynucleotides (e.g. the PLM Polynucleotides of Table 4, 5, or 6).

A “probe” to which a particular polynucleotide molecule specifically hybridizes according to the invention contains a complementary genomic polynucleotide sequence. The nucleotide sequences of the probes can be about 10-200 nucleotides in length. The probes can be genomic sequences of a species of organism, such that a plurality of different probes is present, with complementary sequences capable of hybridizing to the genome of such a species of organism. In other embodiments, the probes are about 10-30, 10-40, 20-50, 40-80, 50-150, 80-120 nucleotides in length, and in particular about 60 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridizae to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684).

In an embodiment, microarrays of the present invention are produced by synthesizing polynucleotide probes on a support wherein the nucleotide probes are attached to the support covalently at either the 3′ or the 5′ end of the polynucleotide.

The invention provides micro-arrays comprising a disclosed marker set. In one embodiment, the invention provides a micro-array for distinguishing pre-term samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality consisting of at least 5, 10, 15, or 20 of the genes corresponding to the markers listed in Table 2, 3, 4, 5, and/or 6 or SEQ ID Nos. 1 through 232. An aspect of the invention provides micro-arrays comprising at least 20, 25, 30, 35, 40, 45, 50, 75, 100, 125, 150, 175, 200, 225, 250, 275, or 300 of the marker genes listed in Table 2, 3, 4, 5 and/or 6 or SEQ ID Nos. 1 through 232. In a particular embodiment, a micro-array comprises the genes listed in Tables 2, 3, 4, 5, and 6, or SEQ ID Nos. 1 through 232.

The invention provides gene marker sets that distinguish preterm labor and term labor and uses therefor. In an aspect, the invention provides a method for classifying a sample as pre-term labor comprising detecting a difference in the expression of a first plurality of genes relative to a control, the first plurality of genes consisting of at least 5, 10, 15, or 20, of the genes corresponding to the markers listed in Table 2, 3, 4, 5, and/or 6, or SEQ ID Nos. 1 through 232. In specific aspects, the plurality of genes consists of at least 20, 25, 30, 35, 40, 45, 50, 75, 100, 125, 150, 175, 200, 225, 250, 275, or 300 of the gene markers listed in Table 2, 3, 4, 5, and/or 6, or SEQ ID Nos. 1 through 232. In another specific aspect, the control comprises nucleic acids derived from a pool of samples from individual term patients.

The invention provides a method for classifying a sample as pre-term labor by calculating the similarity between the expression of at least 5, 10, 15, 20, 25, 30, 40, or 50 of the markers listed in Table 2, 3, 4, 5, and/or 6, or SEQ ID Nos. 1 through 232, in the sample to the expression of the same markers in a term pool, comprising the steps of:
(a) labeling nucleic acids derived from a sample, with a first fluorophore to obtain a first pool of fluorophore-labeled nucleic acids;
(b) labeling with a second fluorophore a first pool of nucleic acids derived from two or more preterm samples, and a second pool of nucleic acids derived from two or more term samples;
(c) contacting the first fluorophore-labeled nucleic acid and the first pool of second fluorophore-labeled nucleic acid with a first micro-array under conditions such that hybridization can occur, and contacting the first fluorophore-labeled nucleic acid and the second pool of second fluorophore-labeled nucleic acid with a second microarray under conditions such that hybridization can occur, detecting at each of a plurality of discrete loci on the first microarray a first flourescent emission signal from the first fluorophore-labeled nucleic acid and a second fluorescent emission signal from the first pool of second fluorophore-labeled genetic matter that is bound to the first microarray and detecting at each of the marker loci on the second microarray the first fluorescent emission signal from the first fluorophore-labeled nucleic acid and a third fluorescent emission signal from the second pool of second fluorophore-labeled nucleic acid;
(d) determining the similarity of the sample to the term and preterm pools by comparing the first fluorescence emission signals and the second fluorescence emission signals, and the first emission signals and the third fluorescence emission signals; and
(e) classifying the sample as preterm where the first fluorescence emission signals are more similar to the second fluorescence emission signals than to the third fluorescent emission signals, and classifying the sample as term where the first fluorescence emission signals are more similar to the third fluorescence emission signals than to the second fluorescent emission signals, wherein the first microarray and the second microarray are similar to each other, exact replicas of each other, or are identical, and wherein the similarity is defined by a statistical method such that the sample and control are similar where the p value of the similarity is less than 0.01, more particularly less than 0.001.

In an embodiment, the array can be used to monitor the time course of expression of one or more PLM Polynucleotides in the array. This can occur in various biological contexts such as progression of pre-term labor.

Arrays are also useful for ascertaining differential expression patterns of PLM Polynucleotides as described herein, and optionally other markers, in normal and abnormal samples. This may provide a battery of nucleic acids that could serve as molecular targets for diagnosis or therapeutic intervention.

Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of pre-term labor or stage or type of pre-term labor in a subject may be determined by (a) contacting a sample from the subject with a binding agent; (b) detecting in the sample a level of PLM polypeptide(s) that binds to the binding agent; and (c) comparing the level of PLM Polypeptide(s) with a predetermined standard or cut-off value.

In particular embodiments of the invention, the binding agent is an antibody. Antibodies specifically reactive with one or more PLM Marker, or derivatives, such as enzyme conjugates or labeled derivatives, may be used to detect one or more PLM Marker in various samples (e.g. biological materials). They may be used as diagnostic or prognostic reagents and they may be used to detect abnormalities in the level of expression of one or more PLM Marker, or abnormalities in the structure, and/or temporal, tissue, cellular, or subcellular location of one or more PLM Marker. Antibodies may also be used to screen potentially therapeutic compounds in vitro to determine their effects on pre-term labor involving one or more PLM Markers, and other conditions. In vitro immunoassays may also be used to assess or monitor the efficacy of particular therapies.

In an aspect, the invention provides a diagnostic method for monitoring or diagnosing pre-term labor in a subject by quantitating one or more PLM Markers in a biological sample from the subject comprising reacting the sample with antibodies specific for one or more PLM Markers, which are directly or indirectly labeled with detectable substances and detecting the detectable substances. In a particular embodiment of the invention, PLM Markers are quantitated or measured.

In an aspect of the invention, a method for detecting pre-term labor is provided comprising:
(a) obtaining a sample suspected of containing one or more PLM Markers associated with pre-term labor;
(b) contacting said sample with antibodies that specifically bind to the PLM Markers under conditions effective to bind the antibodies and form complexes;
(c) measuring the amount of PLM Markers present in the sample by quantitating the amount of the complexes; and
(d) comparing the amount of PLM Markers present in the samples with the amount of PLM Markers in a control, wherein a change or significant difference in the amount of PLM Markers in the sample compared with the amount in the control is indicative of pre-term labor.

In an embodiment, the invention contemplates a method for monitoring the progression of pre-term labor in an individual, comprising:
(a) contacting antibodies which bind to one or more PLM Markers with a sample from the individual so as to form complexes comprising the antibodies and one or more PLM Markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of pre-term labor in said individual.

The amount of complexes may also be compared to a value representative of the amount of the complexes from an individual not at risk of, or afflicted with, a pre-term labor at different stages. A significant difference in complex formation may be indicative of advanced pre-term labor, or an unfavourable prognosis.

In an embodiment of methods of the invention, the PLM Markers encoded by the polynucleotides in Table 2 is detected in samples and higher levels, in particular significantly higher levels compared to a control (normal) is indicative of pre-term labor.

In a further embodiment of methods of the invention, the PLM Markers encoded by the polynucleotides in Table 3 is detected in samples and lower levels, in particular significantly lower levels compared to a control (normal) is indicative of pre-term labor.

A particular embodiment of the invention comprises the following steps
(a) incubating a biological sample with first antibodies specific for one or more PLM Markers which are directly or indirectly labeled with a detectable substance, and second antibodies specific for one or more PLM Markers which are immobilized;
(b) detecting the detectable substance thereby quantitating PLM Markers in the biological sample; and
(c) comparing the quantitated PLM Markers with levels for a predetermined standard.

The standard may correspond to levels quantitated for samples from control subjects without pre-term labor (normal), with a different stage of pre-term labor, or from other samples of the subject. In an embodiment, increased levels of PLM Markers as compared to the standard may be indicative of pre-term labor. In another embodiment, lower levels of PLM Markers as compared to the standard may be indicative of pre-term labor.

Embodiments of the methods of the invention involve (a) reacting a biological sample from a subject with antibodies specific for one or more PLM Markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more PLM Markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects (e.g. normal) without pre-term labor wherein an increase in PLM Marker levels compared with the control subjects is indicative of pre-term labor.

In further embodiment the quantitated levels are compared to levels quantitated for control subjects (e.g. normal) without pre-term labor wherein a decrease in PLM Marker levels compared with the control subjects is indicative of pre-term labor.

Antibodies may be used in any known immunoassays that rely on the binding interaction between antigenic determinants of one or more PLM Marker and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more PLM Marker in a sample may be accomplished by competitive or non-competitive immunoassay procedures in either a direct or indirect format. Detection of one or more PLM Marker using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

In an embodiment of the invention, an immunoassay for detecting more than one PLM Marker in a biological sample comprises contacting binding agents that specifically bind to PLM Markers in the sample under conditions that allow the formation of first complexes comprising a binding agent and PLM Markers and determining the presence or amount of the complexes as a measure of the amount of PLM Markers contained in the sample. In a particular embodiment, the binding agents are labeled differently or are capable of binding to different labels.

Binding agents (e.g. antibodies) may be used in immunohistochemical analyses, for example, at the cellular and sub-subcellular level, to detect one or more PLM Markers, to localize them to particular cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a pre-term labor is contacted with antibodies, preferably monoclonal antibodies recognizing one or more PLM Markers. The site at which the antibodies are bound is determined by selective staining of the sample by standard immunohistochemical procedures. The same procedure may be repeated on the same sample using other antibodies that recognize one or more PLM Markers. Alternatively, a sample may be contacted with antibodies against one or more PLM Markers simultaneously, provided that the antibodies are labeled differently or are able to bind to a different label.

Antibodies specific for one or more PLM Marker may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more PLM Markers. By way of example, if the antibody having specificity against one or more PLM Markers is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more PLM Markers. Generally, antibodies may be labeled with detectable substances and one or more PLM Markers may be localised in tissues and cells based upon the presence of the detectable substances.

In the context of the methods of the invention, the sample, binding agents (e.g. antibodies specific for one or more PLM Markers), or one or more PLM Markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a PLM Marker may be immobilized using sheep anti-mouse IgG Fc fragment specific antibody coated on the carrier or support.

Where a radioactive label is used as a detectable substance, one or more PLM Marker may be localized by radioautography. The results of radioautography may be quantitated by determining the density of particles in the radioautographs by various optical methods, or by counting the grains.

One or more PLM Marker antibodies may also be indirectly labelled with an enzyme using ligand binding pairs. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for PLM Marker antibody is labeled with an enzyme.

Computer Systems

The analytic methods described herein can be implemented by use of computer systems and methods described below and known in the art. Thus the invention provides computer readable media comprising one or more PLM Markers, and/or PLM Polynucleotides, and optionally other markers (e.g. markers of pre-term labor). “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more PLM Markers, and/or PLM Polynucleotides, and optionally other markers.

A variety of data processor programs and formats can be used to store information on one or more PLM Markers, and/or PLM Polynucleotides, and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention also provides in an electronic system and/or in a network, a method for determining whether a subject has pre-term labor or a pre-disposition to pre-term labor, comprising determining the presence or absence of one or more PLM Markers, and/or PLM Polynucleotides, and optionally other markers, and based on the presence or absence of the one or more PLM Markers, and/or LI Polynucleotides, and optionally other markers, determining whether the subject has pre-term labor, or a pre-disposition to pre-term labor, and optionally recommending a procedure or treatment.

The invention further provides in a network, a method for determining whether a subject has pre-term labor or a pre-disposition to pre-term labor comprising: (a) receiving phenotypic information on the subject and information on one or more PLM Markers, and/or PLM Polynucleotides, and optionally other markers associated with samples from the subject; (b) acquiring information from the network corresponding to the one or more PLM Markers, and/or PLM Polynucleotides, and optionally other markers; and (c) based on the phenotypic information and information on the one or more PLM Markers, and/or PLM Polynucleotides, and optionally other markers, determining whether the subject has pre-term labor or a pre-disposition to pre-term labor; and (d) optionally recommending a procedure or treatment.

The invention still further provides a system for identifying selected records that identify pre-term labor. A system of the invention generally comprises a digital computer; a database server coupled to the computer; a database coupled to the database server having data stored therein, the data comprising records of data comprising one or more PLM Markers, and/or PLM Polynucleotides, and optionally other markers, and a code mechanism for applying queries based upon a desired selection criteria to the data file in the database to produce reports of records which match the desired selection criteria.

In an aspect of the invention a method is provided for detecting cells or tissues associated with pre-term labor using a computer having a processor, memory, display, and input/output devices, the method comprising the steps of:
(a) creating records of one or more PLM Markers, and/or PLM Polynucleotides, and optionally other markers, identified in a sample suspected of containing PLM Markers, and/or PLM Polynucleotides associated with pre-term labor;
(b) providing a database comprising records of data comprising one or more PLM Markers, and/or PLM Polynucleotides, and optionally other markers of pre-term labor; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are associated with pre-term labor.

The invention contemplates a business method for determining whether a subject has pre-term labor or a pre-disposition to pre-term labor comprising: (a) receiving phenotypic information on the subject and information on one or more PLM Markers, and/or PLM Polynucleotides, and optionally other markers, associated with samples from the subject; (b) acquiring information from a network corresponding to one or more PLM Markers, and/or PLM Polynucleotides, and optionally other markers; and (c) based on the phenotypic information, information on one or more PLM Markers, and/or PLM Polynucleotides encoding the markers, and optionally other markers, and acquired information, determining whether the subject has pre-term labor or a pre-disposition to a pre-term labor; and (d) optionally recommending a procedure or treatment.

In an aspect of the invention, the computer systems, components, and methods described herein are used to monitor pre-term labor or determine the stage or type of pre-term labor.

Screening Methods

The invention also contemplates methods for evaluating test agents or compounds for their ability to prevent, inhibit or reduce pre-term labor, potentially contribute to pre-term labor, or inhibit or enhance a type of pre-term labor. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting pre-term labor or onset of pre-term labor in a patient, the method comprising comparing:
(a) levels of one or more PLM Markers, and/or PLM Polynucleotides, and optionally other markers in a first sample obtained from a patient and exposed to the test agent; and
(b) levels of one or more PLM Markers and/or PLM Polynucleotides, and optionally other markers in a second sample obtained from the patient, wherein the sample is not exposed to the test agent, wherein a significant difference in the levels of expression of one or more PLM Markers, and/or PLM Polynucleotides, and optionally the other markers, in the first sample, relative to the second sample, is an indication that the test agent is potentially efficacious for inhibiting pre-term labor or onset of pre-term labor in the patient.

The first and second samples may be portions of a single sample obtained from a patient or portions of pooled samples obtained from a patient.

In an aspect, the invention provides a method of selecting an agent for inhibiting pre-term labor or onset of pre-term labor in a patient comprising:
(a) obtaining a sample from the patient;
(b) separately maintaining aliquots of the sample in the presence of a plurality of test agents;
(c) comparing one or more PLM Markers, and/or PLM Polynucleotides, and optionally other markers, in each of the aliquots; and
(d) selecting one of the test agents which alters the levels of one or more PLM Markers, and/or PLM Polynucleotides, and optionally other markers in the aliquot containing that test agent, relative to other test agents.

Still another aspect of the present invention provides a method of conducting a drug discovery business comprising:
(a) providing one or more methods or assay systems for identifying agents that inhibit, prevent or reduce pre-term labor, onset of pre-term labor, or affect a stage or type of pre-term labor in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to pre-term labor or onset of pre-term labor comprising:
(a) maintaining separate aliquots of cells or tissues from a patient with pre-term labor in the presence and absence of the test compound; and
(b) comparing one or more PLM Markers, and/or PLM Polynucleotides, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to pre-term labor or onset of pre-term labor.

Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific PLM Marker, PLM Polynucleotide, or binding agent (e.g. antibody) described herein, which may be conveniently used, e.g., in clinical settings to screen and diagnose patients and to screen and identify those individuals exhibiting a predisposition to developing pre-term labor.

In an embodiment, a container with a kit comprises a binding agent as described herein. By way of example, the kit may contain antibodies or antibody fragments which bind specifically to epitopes of one or more PLM Markers, and optionally other markers, antibodies against the antibodies labelled with an enzyme, and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more markers encoding polynucleotides listed in Table 2, 3, 4, 5, and/or 6, or SEQ ID Nos. 1 through 232, and means for detecting binding of the antibodies to their epitope associated with pre-term labor, either as concentrates (including lyophilized compositions), which may be further diluted prior to use or at the concentration of use, where the vials may include one or more dosages.

A kit may be designed to detect the level of polynucleotides encoding one or more PLM Polynucleotides in a sample. In an embodiment, the polynucleotides encode one or more polynucleotides listed in Table 2, 3, 4, 5 and/or 6, or SEQ ID Nos. 1 through 232. Such kits generally comprise at least one oligonucleotide probe or primer, as described herein, that hybridizes to a PLM Polynucleotide. Such an oligonucleotide may be used, for example, within a PCR or hybridization procedure.

The invention provides a kit containing a micoarray described herein ready for hybridization to target PLM Polynucleotides, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting pre-term labor or onset of pre-term labor in a patient. The kit comprises reagents for assessing one or more PLM Markers or PLM Polynucleotides, and optionally a plurality of test agents or compounds.

The invention contemplates a kit for assessing the presence of cells and tissues associated with pre-term labor or onset of pre-term labor, wherein the kit comprises antibodies specific for one or more PLM Markers, or primers or probes for PLM Polynucleotides, and optionally probes, primers or antibodies specific for other markers associated with pre-term labor (e.g. fibronectin).

Additionally the invention provides a kit for assessing the potential of a test compound to contribute to pre-term labor. The kit comprises cells and tissues associated with pre-term labor or onset of pre-term labor and reagents for assessing one or more PLM Markers, PLM Polynucleotides, and optionally other markers associated with pre-term labor.

Therapeutic Applications

One or more PLM Markers may be targets for immunotherapy. Immunotherapeutic methods include the use of antibody therapy. In one aspect, the invention provides one or more PLM Marker antibodies that may be used to prevent onset of pre-term labor associated with the marker. In another aspect, the invention provides a method of preventing, inhibiting or reducing pre-term labor or the onset of pre-term labor, comprising administering to a patient an antibody which binds specifically to one or more PLM Markers in an amount effective to prevent, inhibit, or reduce pre-term labor or the onset of pre-term labor.

The methods of the invention contemplate the administration of single PLM Marker antibodies as well as combinations, or “cocktails”, of different individual antibodies such as those recognizing different epitopes of other markers. Such cocktails may have certain advantages inasmuch as they contain antibodies that bind to different epitopes of PLM Markers and/or exploit different effector mechanisms. Such antibodies in combination may exhibit synergistic therapeutic effects. In addition, the administration of one or more PLM Marker specific antibodies may be combined with other therapeutic agents. The PLM Marker specific antibodies may be administered in their “naked” or unconjugated form, or may have therapeutic agents conjugated to them.

The PLM Marker specific antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

One or more PLM Marker specific antibody formulations may be administered via any route capable of delivering the antibodies to the site or injury. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the pre-term labor, stage of pre-term labor, the binding affinity and half life of the antibodies used, the degree of PLM Marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more PLM Marker antibodies is also possible and may have advantages in certain situations.

Patients may be evaluated for markers in order to assist in the determination of the most effective dosing regimen and related factors. The assay methods described herein, or similar assays, may be used for quantitating PLM Marker levels in patients prior to treatment. Such assays may also be used for monitoring throughout therapy, and may be useful to gauge therapeutic success in combination with evaluating other parameters such as levels of PLM Markers.

PLM Polynucleotides associated with pre-term labor can be turned off by transfecting a cell or tissue with vectors that express high levels of a desired PLM Polynucleotide. Such constructs can inundate cells with untranslatable sense or antisense sequences. Even in the absence of integration into the DNA, such vectors may continue to transcribe RNA molecules until all copies are disabled by endogenous nucleases.

Vectors derived from retroviruses, adenovirus, herpes or vaccinia viruses, or from various bacterial plasmids, may be used to deliver PLM Polynucleotides to a targeted organ, tissue, or cell population. Methods well known to those skilled in the art may be used to construct recombinant vectors that will express PLM Polynucleotides such as antisense. (See, for example, the techniques described in Sambrook et al (supra) and Ausubel et al (supra).)

Methods for introducing vectors into cells or tissues include those methods discussed herein and which are suitable for in vivo, in vitro and ex vivo therapy. For example, delivery by transfection and by liposome are well known in the art.

Modifications of gene expression can be obtained by designing antisense molecules, DNA, RNA or PNA, to the regulatory regions of a PLM Polynucleotide, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of PLM Polynucleotides.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

One or more PLM Markers and PLM Polynucleotides (e.g. down-regulated PLM Markers and PLM Polynucleotides), and fragments thereof, and compounds or agents identified using a method of the invention may be used to prevent, treat, or reduce pre-term labor or onset of pre-term labor in a subject. The markers or polynucleotides may be formulated into compositions for administration to subjects with a pre-disposition for or suffering from pre-term labor. Therefore, the present invention also relates to a composition comprising one or more PLM Markers or PLM Polynucleotides, or a fragment thereof, and a pharmaceutically acceptable carrier, excipient or diluent. A method for treating or preventing pre-term labor in a subject is also provided comprising administering to a patient in need thereof, one or more PLM Markers or PLM Polynucleotides, an agent or compound identified using a method of the invention, or a composition of the invention.

The invention further provides a method of preventing, inhibiting, or reducing pre-term labor in a patient comprising:
(a) obtaining a sample comprising tissue or cells associated with or diagnostic for pre-term labor from the patient;
(b) separately maintaining aliquots of the sample in the presence of a plurality of test agents;
(c) comparing levels of one or more PLM Markers, and/or PLM Polynucleotides in each aliquot;
(d) administering to the patient at least one of the test agents which alters the levels of the PLM Markers, and/or PLM Polynucleotides in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. antenatal glucocorticoids or tocolysis). The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The methods of the invention for use on subjects/individuals/patients contemplate prophylactic as well as therapeutic or curative use. Typical subjects for treatment include persons susceptible to, suffering from or that have suffered pre-term labor. In embodiments of the invention, the methods and compositions described herein are used prophylactically to prevent development of pre-term labor.

The following non-limiting example is illustrative of the present invention:

### EXAMPLE

A cDNA micro-array analysis was used to define gene expression profiles from peripheral blood leukocytes of symptomatic women in threatened pre-term labor. A novel “non-biased” approach was used that surveys the whole genome for potential markers of imminent pre-term birth. The cDNA micro-array is a non-biased screen of the entire genome or at least the number of genes represented on the cDNA chipset (for example, 19,200 expressed sequence tags—University Health Network human single spot 19Kv7 micro-array).

This technique makes no predictions as to which gene or more likely gene clusters expressed by maternal mononuclear leukocytes will be predictive of imminent pre-term delivery. For the development of a diagnostic test there is also no requirement that the role of the gene product be known or even that it be identified beyond its sequence within the genome database, merely that the marker predicts imminent pre-term delivery with a high degree of sensitivity and specificity. In many ways this is an ideal use of micro-array technology. Moreover, unlike fetal fibronectin, this unbiased approach has the potential to discriminate between pre-term labors resulting from different etiologies (e.g. infection vs. fetal stress vs. myometrial activation) based on the differential gene expression profiles that might be induced. This may ultimately provide the opportunity for the provision of an individualized therapeutic strategy that is likely to be a more effective approach to the management of pre-term labor.

A diagnostic test based on the gene expression profiles identified in the peripheral blood leukocytes of symptomatic women in threatened pre-term labor will have a better ability to predict imminent pre-term birth ability than fetal fibronectin for a number of reasons. First, it is likely that a diagnostic test based on an expression profile of a specifically selected gene cluster will have a higher positive predictive value than that reported with fetal fibronectin (14%) when it is assessed in symptomatic women in threatened pre-term labor. Complex statistical analysis and predictive modeling will allow the selection of a gene cluster that possesses the best combination of sensitivity, specificity, and positive and negative predictive values for the dataset available from the study. Second, a test based on a maternal blood sample is unlikely to have the multiple contraindications that are a practical issue with FFN testing (cervical dilation greater than 3 cm, pre-term premature rupture of fetal membranes, moderate or heavy bleeding, cervical examination, coitus or transvaginal ultrasound within the previous 24 hours, obstetric cream or KY jelly) which preclude its use in up to 80% of women who present to tertiary centers with threatened pre-term labor.

Rationale for Mononuclear Leukocytes as a Diagnostic Marker of True Pre-Term Labor

While the molecular information required to diagnose true pre-term labor likely resides within the myometrium itself, an acceptable diagnostic test must use a more non-invasive approach. The source of RNA for the micro-array analysis is maternal peripheral blood cells, essentially mononuclear leukocytes. Peripheral leukocytes can be used to monitor a variety of pathophysiologic situations, including the progression of labor (62) and myometrial responsiveness to β-adrenergic tocolysis (63). Mononuclear leukocytes from women in active labor exhibit a significant attenuation of β-adrenergic receptor function due to reduced adenyl cyclase activity. This effect could be induced in mononuclear leukocytes from non-laboring women by pre-incubation with PGE but not oxytocin or PGF. It has been shown that, (1) mononuclear leukocyte and myometrial β-receptor number are positively correlated, (2) the mechanism responsible for desensitization of mononuclear leukocytes from pregnant women involves a down-regulation of the β-receptor with post receptor mechanisms remaining fully functional, (3) the process of mononuclear leukocyte desensitization can be monitored temporally during administration of tocolytic therapy to women.

Evaluation of Ten Women Presenting with Threatened Pre-Term Labour:

Method

Blood samples were collected from 10 women presenting in threatened pre-term labour (T-PTL) between 24-36 weeks gestation. Threatened pre-term labour was defined as regular uterine contractions, cervical dilation 0-4 cm, intact fetal membranes, and a clinical decision to treat the mothers with corticosteroids and tocolytic medication. RNA was extracted (PAXgene blood RNA extraction kit; Qiagen), reverse transcribed to cDNA and labelled using an indirect amino-allyl protocol. To control for variation, 4 micro-arrays (human 19K cDNA, University Health Network, Toronto) were prepared on each patient comparing samples to human universal reference RNA (Stratagene). Gene expression data was analyzed using Axon Genepix 4000A, and Vector Xpression (v3.1.0) software was used for cluster analysis. Results: Of the 10 women with T-PTL, 5 progressed to pre-term delivery within 48 hours; the remaining 5 delivered at term gestations (p=0.004). Demographic data of the two groups of women is presented in Table 1.

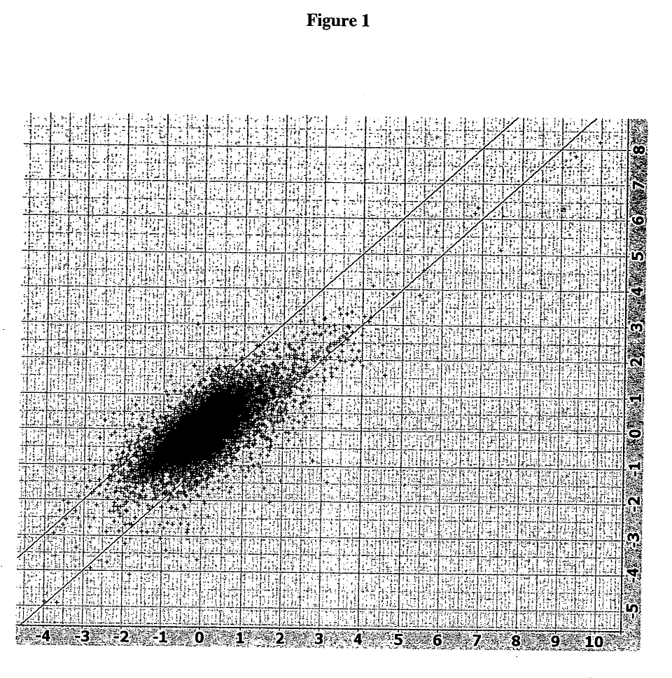

The 19200 spots on the micro-array lead to data on 18879 expression sequence tags (EST's) that correspond to approximately 15000 known genes, and 4000 unknown mRNA sequences. When the gene expression profiles of leukocytes from women with T-PTL who progressed to delivery within 48 hours (delivered group) were compared to women with T-PTL whose pregnancies continued to term (undelivered group), there were significant differences in gene expression between the two groups (FIG. 1). The leukocytes of the women who progressed rapidly to delivery demonstrated up regulation of 266 EST's (range 2-9 fold increase) and a down regulation of 561 EST's (range 2-25 fold). The lists of EST's and their corresponding genes are in Tables 2 and 3. These two lists of EST's can form the basis of the custom “pre-term labour micro-array”.

Evaluation of gene ontology of the known genes from the lists of up and down-regulated ESTs predicted the following functions: cell growth and maintenance (44%); protein metabolism (31%); nucleic acid metabolism (25%); signal transduction (19%); and cell death (12.5%).

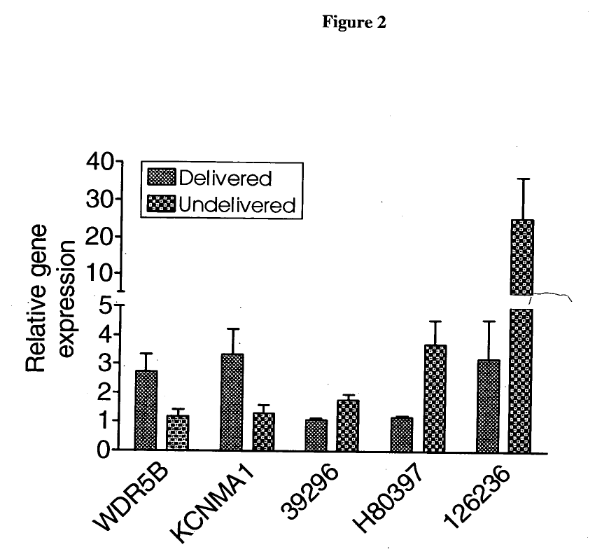

When statistical analysis of the gene expression was undertaken comparing the two groups (delivered vs. undelivered), there were 5 EST's where the difference in gene expression between the two groups was significant at p<0.001 (Table 5) and 44 EST's where the magnitude of the difference between the two groups was P<0.01 (Table 4). Of the 5 most highly significant EST's, 2 are known genes 3 are unknown EST's. The gene expression of these 5 EST's is presented in FIG. 2. The EST's are in Table 5.

The 5 most differentially expressed EST's are presented in FIG. 2. WDR5B is a gene that codes for a protein involved in protein-protein interactions. KCNMA1 is the gene for the MaxiK channels, which are large conductance, voltage and calcium sensitive potassium channels that are fundamental to the control of smooth muscle tone and neuronal excitability.

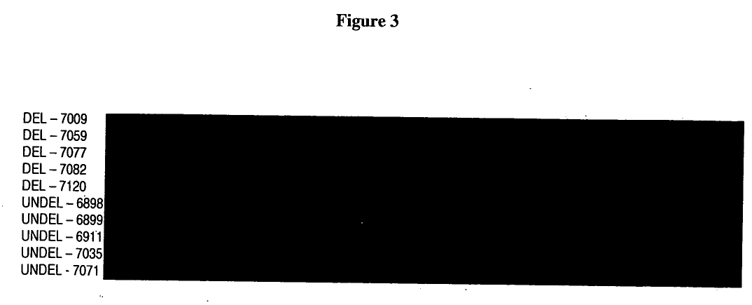

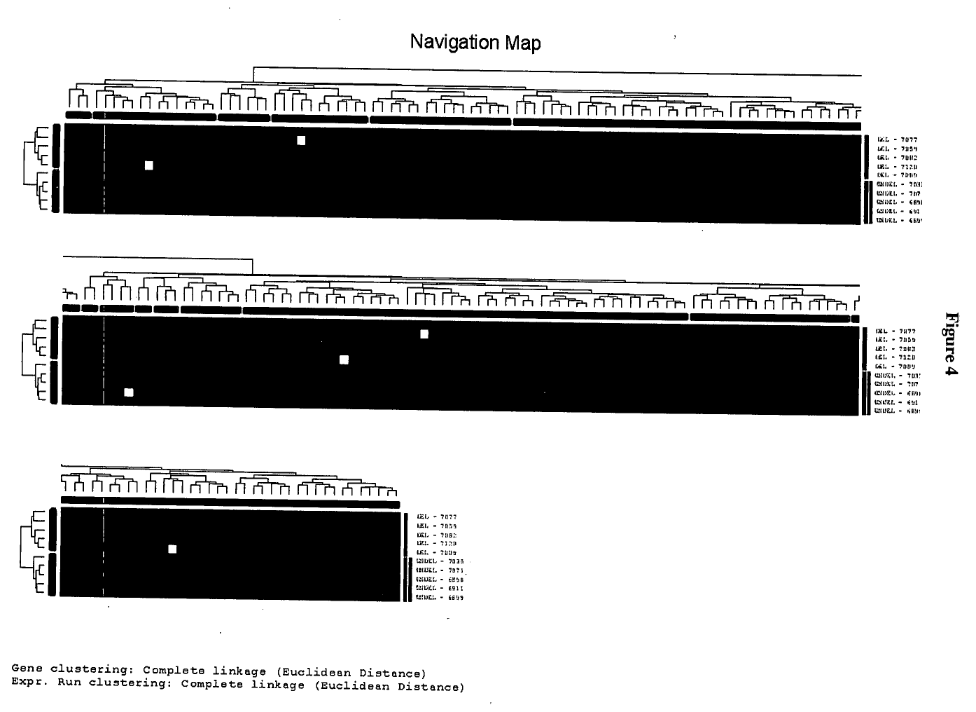

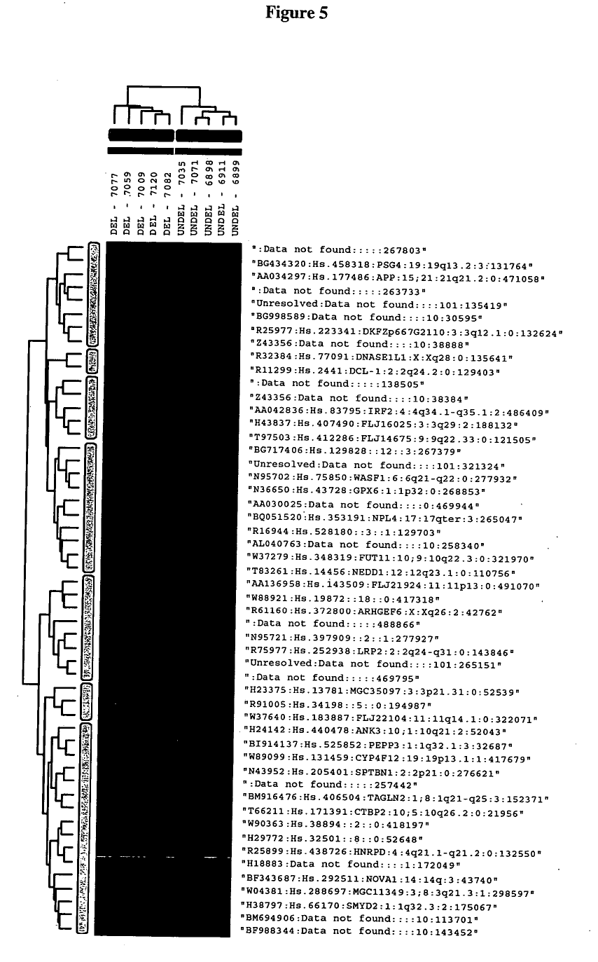

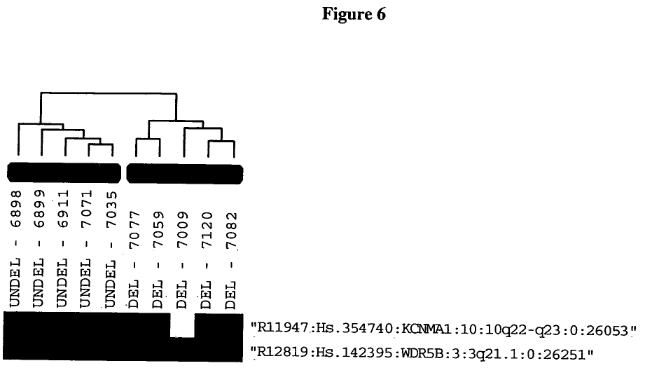

Evaluation of the gene expression profiles of each of the 10 women in this study identified a clear difference between the two outcome groups. This difference is clearly visible from the gene expression profiles of the ten women in the two study groups (FIG. 3). Both hierarchical and non-hierarchical unsupervised cluster analysis has been performed on the gene expression profiles obtained from the 10 women in this study. Subsets of EST's ranging from 2 to 216 EST's are capable of successfully clustering the gene expression profiles into delivered and undelivered groups. The ability to successfully predict this important outcome occurs at the first division of the dendogram and is reproducible using a variety of clustering techniques including analysis based on complete linkage of Euclidean distance, K-mean divisive, Batch K-mean and Batch 1d-SOM clustering algorithms. Examples of the cluster analysis are provided in FIG. 4 to FIG. 6 and the relevant EST's are listed in Table 6.

Biologic Rationale

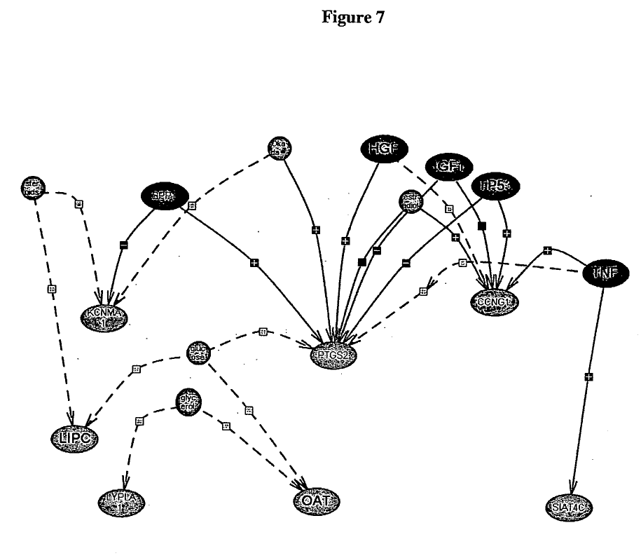

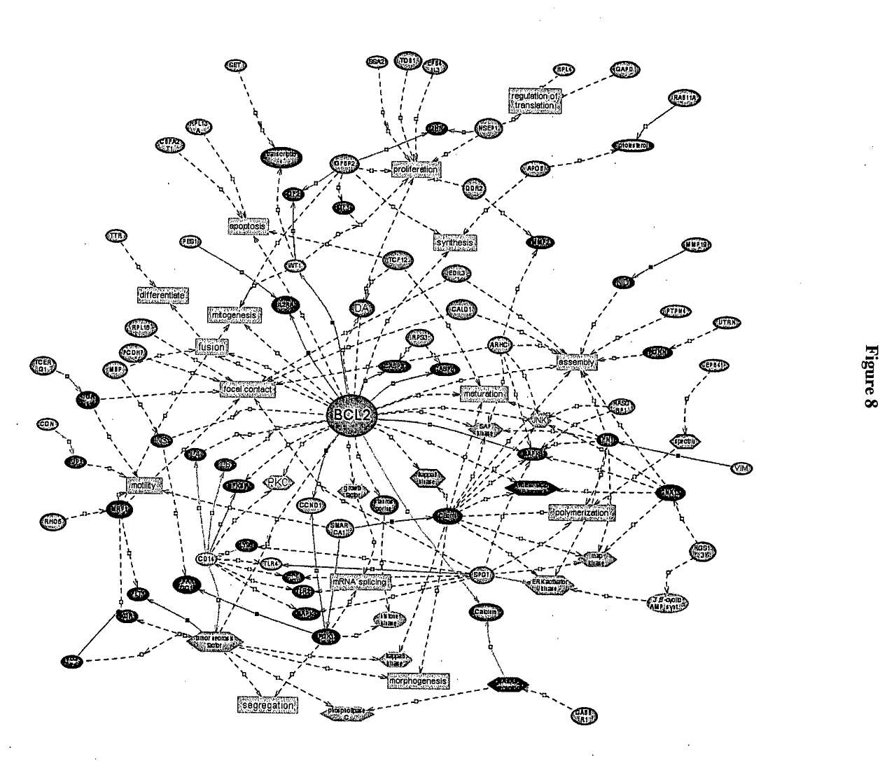

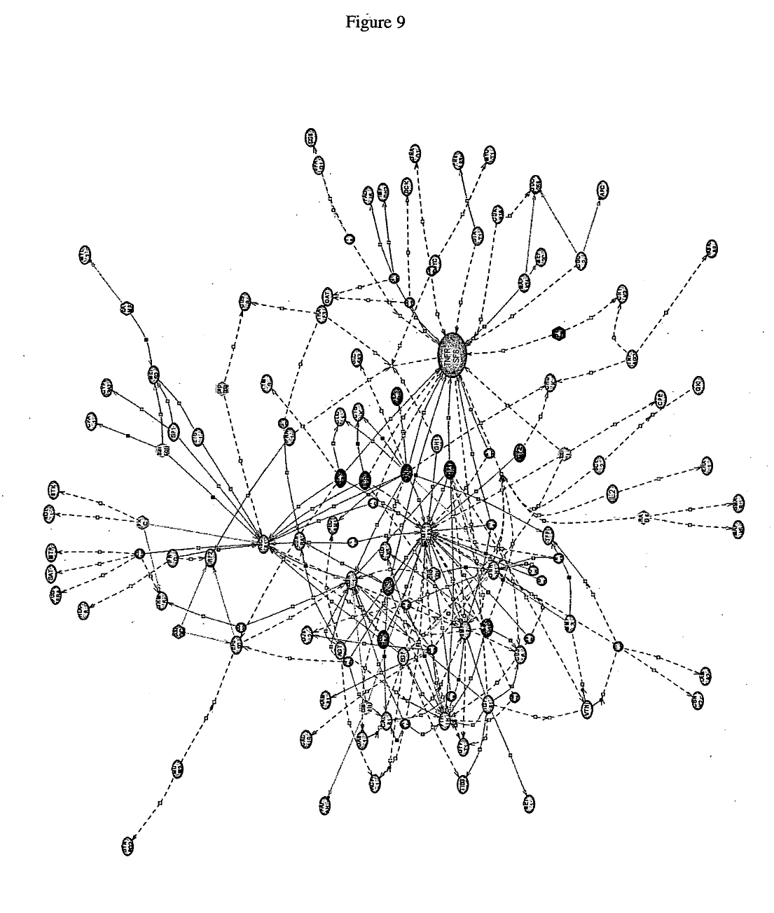

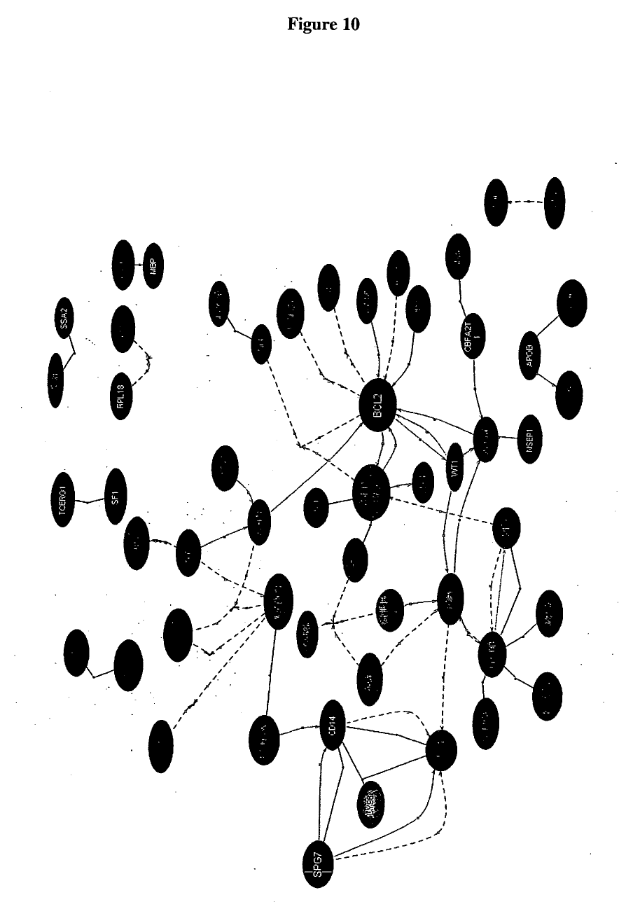

To further evaluate the biologic plausibility of this novel approach, pathway analysis was undertaken of the genes associated with the EST's that were different between the two groups of women. Of the 44 EST's that were significantly different between the two groups of women (p<0.01), 20 are currently known genes. Pathway analysis of common regulators of these 20 genes developed a pathway (FIG. 7) centering on PTGS2D, the gene coding for cyclo-oxygenase 2 (COX-2), which is known to be central to the prostaglandin activation that occurs during labour. Further, pathway analysis of common targets of the genes whose expression was increased greater than 2 fold in the women with T-PTL (FIG. 8) who progressed to delivery within 48 hours (as compared to those who delivered at term) developed a pathway around BCL2. B-cell CLL/lymphoma 2 codes for a protein that is an integral inner mitochondrial membrane protein that blocks apoptotic death of some cells such as lymphocytes. Similarly, pathway analysis of regulators of the genes whose expression was decreased greater than 2 fold in the women with T-PTL who progressed to delivery centres on apoptosis and cell proliferation (FIG. 9). The two nodes with the greatest number of links to other down regulated genes are TNFR-SF6 and IGFBP3. Tumour necrosis factor receptor super family (member 6) has been shown to play a central role in the physiological regulation of programmed cell death and insulin-like growth factor binding protein 3 plays a key role in regulating cell proliferation and apoptosis. The combined pathway (including both up and down regulated genes) results in a network that suggests a net down regulation of apoptosis and a net increase in genes associated with proliferation in leukocytes in women with T-PTL that progress to delivery (FIG. 10). The centering of the pathways around genes that block apoptotic cell death is consistent with the animal literature that has previously demonstrated reduced apoptosis in leukocytes of cows during labour.

Methods

Patient Recruitment:

All clinical samples for phase one were collected at Mount Sinai Hospital, Toronto. Blood samples were analyzed from women who present with symptoms of threatened idiopathic pre-term labour (approximately 20 who progress to pre-term delivery and 20 whose pregnancies continue to term). To select a homogenous group of women with idiopathic pre-term labour, detailed inclusion and exclusion criteria were determined. The inclusion criteria include: 1) 24 to 37 week's gestation; 2) regular uterine contractions; 3) cervical dilation <4 cm; 4) intact fetal membranes. The exclusion criteria include: 1) antepartum haemorrhage [abruptio placenta, placenta praevia]; 2) pre-term pre-labour rupture of membranes; 3) clinical chorioamnionitis [febrile (>37.5° C.), uterine tenderness, mother systemically unwell, fetal tachycardia]; 4) fetal anomaly; 5) preeclampsia; 6) intrauterine growth restriction; 7) diabetes or gestational diabetes; 8) maternal medical condition; 9) multi-fetal pregnancy.

Sample Processing

Detailed maternal data was collected from the clinical record. Maternal blood samples (12.5 mL) was collected into five PAXgene blood collection tubes (Qiagen, Mississauga, Ontario, Canada) designed specifically for the extraction of RNA from whole blood. Preliminary studies determined that approximately 53 μg of high quality RNA (OD 260:280 ratio 2.03±0.22 {mean±SD}) can be extracted from each 12.5 mL maternal blood sample. This amount was adequate to 1) prepare four micro-arrays on each sample, and 2) to confirm results using real time polymerase chain reaction (RT-PCR). RNA extraction samples were concentrated with MinElute columns (Qiagen, Mississauga, Ontario, Canada) to obtain optimum RNA concentrations for micro-array analysis and their integrity was assessed using the Agilent 2100 Bioanalyser system prior to micro-array preparation.

Maternal RNA samples from each woman in this study were used to generate four micro-arrays (i.e. 4 technical replicates). To control for variations during reverse transcription, four separate reverse transcriptions were performed. On each array the sample was compared to human universal reference RNA (Stratagene, Calif., USA). An indirect (amino-allyl) labeling protocol was utilized to reduce dye incorporation bias. Dye swapping was performed on one of the four micro-arrays performed on each sample to further reduce bias induced by variable dye incorporation. The micro-array protocol used in the study was validated. In brief, cDNA was prepared by reverse transcription of both sample RNA and reference RNA using amino allyl labeled dUTP. After purification of the labeled cDNA (Qiagen PCR purification kit), cDNA was resuspended in individually prepared Cy3 and Cy5 aliquots as appropriate (GE Healthcare, Quebec, Canada). The reaction was then quenched and unincorporated Cy dyes was removed (Qiagen PCR purification kit). Labeled sample cDNA and reference cDNA was then combined and hybridized onto the micro-arrays. After incubation overnight in hybridization chambers (BioRad, Mississauga, Ontario, Canada) the arrays were washed, dried and the fluorescent signals on the micro-array were imaged and scanned (Axon Genepix 4000A). Subsequently, a ratio of the fluorescence of the two fluors was obtained for each DNA spot on the array. The principal behind this approach was that this ratio was proportional to the relative expression of that RNA species in the tissue sample. Since human universal reference RNA was used as a control for each chip, ratios (relative RNA expression) could be compared across all chips. Statistical algorithms and pattern recognition techniques were then applied to ratio data to identify gene or gene clusters that were specific to experimental endpoints.

Primary Outcome Measures

The primary outcome measures in the study were relative measures of gene expression compared to gestation at delivery. The four time points for delivery that were considered in the study were: 1) delivery within 48 hours of clinical presentation; 2) delivery within 7 days of clinical presentation; 3) delivery prior to 34 weeks gestation; and 4) delivery prior to 37 weeks gestation. The first two time points (delivery within 48 hours and 7 days) were selected as they are clinically relevant for decision making relating to corticosteroid therapy and transfer to tertiary centers for potential delivery. The second two time points (delivery prior to 34 and 37 weeks) were considered to enable a direct comparison with previously published data relating to the predictive value of fetal fibronectin testing which reported predictive values for all four of the time points that were considered in the study.

Validation of Micro-Array Data:

A large number of genes (half up-regulated and half down-regulated) were identified with significantly different gene expression when compared to samples from women who deliver pre-term with samples from women whose pregnancies continue. From this subset of genes up to 50 genes can be identified where expression patterns reliably predict the timing of delivery. To validate the data obtained from the micro-array studies, real time polymerase chain reaction (RT-PCR) will be performed on up to 50 selected genes. The genes selected for RT-PCR confirmation will be those with the greatest ability to predict the outcome groups.

Complementary DNA is synthesized from each maternal RNA sample using the TaqMan Reverse Transcription Kit (Applied Biosystems, Foster City, Calif., USA). The total volume of the reverse transcription reaction is 100 μl, which contains 10 μl of 10× TaqMan RT buffer, 22 μl of 25 mM MgCl₂, 20 μl of 2.5 mM deoxyNTPs mixture, 5 μl of 50 uM random hexamers, 2 μl of 20 U/L RNase inhibitor, 2.5 ul of 50 U/ul MultiScribe Reverse Transcriptase, 26.5 μl of RNase-free water, and 10 μl of 100 mM DTT (Invitrogen, GmbH, Karlsruhe, Germany). To this mixture, 2 μg of total RNA is added for conversion into cDNA. The thermal cycling conditions include primer incubation step for 10 min at 25° C., reverse transcription for 30 min at 48° C., and reverse transcription inactivation for 5 min at 95° C.

Gene-specific primers are then designed using Primer Express (Applied Biosystems) to fulfill all criteria for real-time PCR primers. PCR is performed in an optical 96-well plate with an ABI PRISM 7900 HT Sequence Detection System (Applied Biosystems), using the SYBR Green detection chemistry. Reaction conditions for each gene including primer and template concentrations, and thermal profile will be optimized. Each reaction contains 12.5 μl of 2×SYBR Green master mix, optimized amount of primers and cDNA concentration, and water to make up a total reaction volume of 25 μl. A general thermal profile is 95° C. for 10 min, 40 cycles of 95° C. for 15 sec and 60° C. for 1 min. After PCR, a dissociation curve is constructed by increasing temperature from 65° C. to 95° C. for detection of PCR product specificity.

Data will be analyzed using the SDS 2.1 software (Applied Biosystems). The relative standard curve or the delta Ct method will be used for data analysis, depending on the number of genes that display significant expression changes from micro-array.

Development of the Custom Pre-Term Labor Micro-Array

The “pre-term birth genetic signature” determined as described herein will be the basis for the development of a custom pre-term labor micro-array. Custom micro-arrays can be produced using both cDNA and oligonucleotide technology. These are currently being produced by a large number of both commercial and academic institutions.

When developing a custom micro-array to validate a “gene expression signature” for particular conditions it is important that the number of genes represented on the array is not overly restrictive. The subset of genes for the custom array should include: all of the sequences with the highest ability to discriminate outcomes; all of the sequences with significant changes in expression (≧2-fold change) identified comparing the experimental groups and; an adequate sample of sequences (approximately one third of the total) where there is no change in expression between the study groups to enable reliable normalization and interpretation of data.

The custom pre-term labour micro-arrays can be used to investigate gene-environment interactions in pre-term birth. In addition the custom micro-array will have the ability to potentially discriminate between pre-term labors resulting from different etiologies (e.g. infection vs. fetal stress vs. myometrial activation) based on the differential gene expression profiles that might be induced. This may ultimately provide the opportunity for the provision of an individualized therapeutic strategy that is likely to be a more effective approach to the management of pre-term labor.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to the “antibody” is a reference to one or more antibodies and equivalents thereof known to those skilled in the art, and so forth.

Below full citations are set out for the references referred to in the specification.

TABLE 1
Patient demographic data

| | Pre-term delivery | | |
|---|---|---|---|
| | <48 hours | Term delivery | |
| | (n = 5) | (n = 5) | p-value |
| Gravidity | 3 [1-8] | 3 [1-3] | 0.88 |
| Parity | 1 [0-7] | 1 [0-2] | 0.57 |
| Cervical dilation (cm) | 3.0 [2-4] | 1.6 [1-3] | 0.03 |
| Effacement (100%) | 66 [50-100] | 38 [0-70] | 0.19 |
| Previous PTD | 2 | 1 | 1.00 |
| Gestation at presentation | 29.9 [24-36.3] | 31.6 [30.4-32.4] | 0.46 |
| Gestation delivery | 29.9 [24-36.3] | 38.6 [37.4-40.4] | 0.02 |

TABLE 2
2-Fold Upregulated EST's

| Original | Confirmed | Unigene | | | Chromosome |
|---|---|---|---|---|---|
| Clone ID | Clone ID | ID | Gene Name | Chromosome | location |
| 427897 | AA001336 | Hs.122408 | | 3 | |
| 428216 | AA001809 | Hs.469029 | | 2 | |
| 428944 | AA004445 | Hs.75627 | CD14 | 5 | 5q22-q32 |
| 429798 | AA009503 | Hs.446116 | | 13 | |
| 430083 | AA009869 | Hs.458444 | EPB41 | 16; 1 | 1p33-p32 |
| 430327 | AA010522 | Hs.250607 | UTRN | 6 | 6q24 |
| 428644 | AA033929 | Hs.19156 | | 14 | |
| 471830 | AA035749 | Hs.433326 | IGFBP2 | 2; 8 | 2q33-q34 |
| 485076 | AA039258 | Hs.375921 | RPL31 | 10; 12; 14; 18; 2 | 2q12.1 |
| 487358 | AA040656 | Hs.224843 | ZNF502 | 3 | 3p21.32 |
| 486881 | AA043087 | Hs.408747 | SPAG1 | 8 | 8q22.2 |
| 486832 | AA043301 | Hs.437173 | COL4A1 | 13; 2 | 13q34 |
| 486731 | AA044433 | Hs.433024 | | 9 | |
| 486698 | AA044450 | Hs.459562 | | 9 | |
| 488294 | AA088642 | Hs.480392 | | 4 | |
| 489800 | AA098903 | Hs.146246 | MGC45780 | 8 | 8p21.1 |
| 489898 | AA114919 | Hs.74497 | NSEP1 | 13; 14; 15; 1; 7; 9 | 1p34 |
| 491783 | AA115064 | Hs.465453 | | 18 | |
| 503327 | AA128246 | Hs.17834 | DONSON | 21 | 21q22.1 |
| 503441 | AA128257 | | | | |
| 502423 | AA135452 | Hs.444818 | CGGBP1 | 12; 3 | 3p12-p11.1 |
| 502845 | AA135502 | Hs.75618 | RAB11A | 15 | 15q21.3-q22.31 |
| 491169 | AA137140 | Hs.441044 | EDIL3 | 5 | 5q14 |
| 491388 | AA148531 | Hs.439202 | DPP8 | 15 | 15q22 |
| 502932 | AA151344 | Hs.24879 | PPAP2C | 19 | 19p13 |
| 504612 | AA152139 | In multiple clusters | | | |
| 504638 | AA152216 | In multiple clusters | | | |
| 446259 | AA203731 | Hs.434896 | PRODH2 | 19 | 19q13.1 |
| 36886 | AA809790 | Hs.9552 | BART1 | 16; 1 | 16q13 |
| 376316 | AA932087 | | | | |
| 376260 | AI681538 | Hs.293907 | FLJ23403 | 18 | 18p11.21 |
| 113979 | AI768894 | Hs.18376 | CGN | 1 | 1q21 |
| 42613 | AL598331 | | | | |
| 270300 | AL703266 | | | | |
| 502691 | AV682098 | | | | |
| 203939 | AV717735 | | | | |
| 152265 | AW297717 | Hs.5057 | CPD | 17; 19; 8 | 17p11.1-q11.2 |
| 43340 | AW953110 | | | | |
| 310074 | BE256276 | Hs.265174 | RPL32 | 16; 18; 22; 3; 6; 7; 9 | 3p25-p24 |
| 273485 | BE747712 | Hs.4791 | KIAA0376 | 22 | 22q11.23 |
| 36332 | BE879779 | | | | |
| 239053 | BE881603 | | | | |
| 501634 | BF336069 | | | | |
| 111612 | BG219008 | | | | |
| 266815 | BG529617 | Hs.356502 | RPLP1 | 11; 15; 19; 1; 2; 3; 5; | 15q22 |
| | | | | 6; 7; X | |
| 343563 | BG565169 | Hs.433670 | FTL | 10; 19; 1; 20; 21; 4; | 19q13.3-q13.4 |
| | | | | 8; X | |
| 198940 | BG566154 | Hs.387576 | RPS3 | 11; 12; 19; 3; 6; 9 | 11q13.3-q13.5 |
| 116445 | BG621623 | Hs.449631 | HBG1 | 11; 7 | 11p15.5 |
| 503601 | BG776239 | Hs.1145 | WT1 | 11 | 11p13 |
| 139515 | BG822880 | Hs.381184 | RPS19 | 14; 16; 19; 20; 4; 7 | 19q13.2 |
| 32105 | BI756203 | Hs.311765 | SPG7 | 12; 16 | 16q24.3 |
| 35919 | BI761144 | Hs.124940 | RHO6 | 12 | 12q12-q13 |
| 501717 | BI914643 | Hs.293836 | JPH1 | 8 | 8q21 |
| 488202 | BM009849 | Hs.381184 | RPS19 | 14; 16; 19; 20; 4; 7 | 19q13.2 |
| 502705 | BM508191 | Hs.441169 | SLC7A6 | 16 | 16q22.1 |
| 152922 | BM509122 | Hs.458262 | IGL@ | 22 | 22q11.1-q11.2 |
| 115000 | BM678693 | | | | |
| 375896 | BM704546 | | | | |
| 172911 | BM727279 | | | | |
| 195586 | BM783899 | | | | |
| 233938 | BM832831 | | | | |
| 60664 | BM888296 | | | | |
| 61283 | BM907545 | Hs.435800 | VIM | 10; 6 | 10p13 |
| 380727 | BM908669 | Hs.169476 | GAPD | 10; 12; 19; 22; 4; 6; | 12p13 |
| | | | | 8; 9; X | |
| 297656 | BM912969 | Hs.437594 | RPLP2 | 11; 17; 19 | 11p15.5-p15.4 |
| 230433 | BM914621 | Hs.5163 | MGC23280 | 17 | 17q11.2 |
| 501516 | BM931534 | | | | |
| 491648 | BM969982 | | | | |
| 383201 | BM974828 | Hs.409634 | RPL18 | 17; 19; 1; 2 | 19q13 |
| 220049 | BM998885 | | | | |
| 376152 | BQ048895 | Hs.436687 | SET | 10; 12; 16; 1; 3; 9 | 9q34 |
| 234306 | BQ055308 | Hs.478508 | RPL4 | 10; 15; 3; 9 | 15q22 |
| 297421 | BQ055926 | Hs.7517 | MGC4730 | 1; 9 | 9p24.1 |
| 299783 | BQ072807 | Hs.449070 | RPL13A | 10; 12; 13; 14; 19; 1; | 19q13.3 |
| | | | | 21; 2; 7 | |
| 27769 | BQ108662 | Hs.282883 | MGC4251 | 17; 4 | 17q21.31 |
| 149932 | H01247 | Hs.154057 | MMP19 | 12 | 12q14 |
| 45269 | H08285 | Hs.449641 | LOC92170 | 10 | 10q26.3 |
| 47378 | H09086 | Hs.288178 | SSA2 | 1 | 1q31 |
| 48177 | H12180 | Hs.28088 | LOC51308 | 5 | 5q31 |
| 148686 | H12790 | Hs.156304 | ASXL2 | 2 | 2p24.1 |
| 163466 | H14103 | Hs.408543 | MBP | 18; 2 | 18q23 |
| 48404 | H14396 | Hs.239147 | GDA | 9 | 9q21.11-21.33 |
| 159428 | H15023 | Hs.406330 | DKFZp686B2197 | 10; 19 | 19q13.43 |
| 49250 | H15096 | Hs.443020 | PCDH7 | 4 | 4p15 |
| 51976 | H23312 | Hs.107010 | | 12 | |
| 52538 | H23374 | Hs.514719 | | 18; 7 | |
| 162117 | H25740 | In multiple clusters | | | |
| 52738 | H29424 | Hs.288368 | FLJ22728 | 11 | 11p15.2 |
| 190254 | H29950 | Hs.178137 | TOB1 | 17 | 17q21 |
| 190185 | H30816 | Hs.32452 | DKFZp566D234 | 4 | 4q32.3 |
| 163393 | H39539 | Hs.356861 | | 22 | |
| 176371 | H45653 | Hs.179852 | DC-UbP | 5 | 5q35.2 |
| 193546 | H47450 | Hs.177861 | P14 | 2 | 2pter-p25.1 |
| 193713 | H47769 | Hs.523780 | | 1 | |
| 179453 | H51356 | Hs.167017 | GABBR1 | 10; 6 | 6p21.31 |
| 202410 | H52618 | Hs.36679 | | 16 | |
| 207302 | H59667 | Hs.469497 | | 2 | |
| 236155 | H61757 | Hs.129969 | ELK4 | 17; 1 | 1q32 |
| 209388 | H64092 | Hs.131315 | BAL | 3 | 3q13-q21 |
| 211005 | H65772 | Hs.117835 | | 12 | |
| 212090 | H68961 | Hs.499532 | | 10 | |
| 212401 | H69475 | Hs.95111 | FLJ35936 | 18 | 18p11.31-p11.23 |
| 239446 | H70046 | Hs.17165 | RGS13 | 1 | 1q31.1 |
| 232601 | H72582 | Hs.35992 | | 8 | |
| 234598 | H77585 | Hs.271014 | | 12 | |
| 229369 | H79396 | In multiple clusters | | | |
| 239666 | H80492 | Hs.90858 | CBFA2T1 | 8 | 8q22 |
| 239939 | H82031 | Hs.480722 | | 4 | |
| 198975 | H83239 | Hs.406751 | | 12 | |
| 222188 | H83491 | Hs.40507 | | 7 | |
| 222111 | H84877 | Hs.396358 | FLJ11273 | 7 | 7p21.3 |
| 252238 | H87518 | Hs.179735 | ARHC | 1 | 1p13.1 |
| 253560 | H89547 | Hs.290356 | MESDC1 | 15 | 15q13 |
| 240937 | H90997 | Hs.1191 | KIAA0073 | 5 | 5q12.3 |
| 241066 | H91404 | Hs.6083 | | 6 | |
| 253670 | H91586 | Hs.473392 | | 21 | |
| 220695 | H95520 | Hs.152292 | SMARCA1 | 1; X | Xq25 |
| 254418 | N22200 | Hs.119275 | | 7 | |
| 254030 | N22317 | In multiple clusters | | | |
| 261836 | N23578 | Hs.438166 | TCF12 | 15 | 15q21 |
| 266521 | N31180 | Hs.94891 | FLJ22729 | 17 | |
| 267591 | N35730 | Hs.279819 | MAGEH1 | X | Xp11.22 |
| 270548 | N42169 | Hs.293865 | | 7 | |
| 271718 | N43838 | Hs.103839 | EPB41L3 | 18 | 18p11.32 |
| 273918 | N46500 | | | | |
| 233989 | N49986 | Hs.509692 | | 14 | |
| 285487 | N66410 | Hs.167767 | | 9 | |
| 285058 | N67543 | Hs.49352 | | 18 | |
| 255836 | N71721 | Hs.26208 | COL16A1 | 1 | 1p35-p34 |
| 246497 | N73236 | Hs.37636 | C10orf24 | 10 | 10q22.1 |
| 287707 | N75892 | Hs.368672 | | 17 | |
| 289457 | N76698 | Hs.268012 | FACL3 | 2 | 2q34-q35 |
| 290300 | N77571 | Hs.404215 | AP1GBP1 | 17; 4 | 17q21.1 |
| 292865 | N91077 | Hs.27258 | SIP | 12; 1; 2 | 1q24-q25 |
| 308590 | N95419 | Hs.405913 | GRCC10 | 12; 4 | 12p13.31 |
| 294997 | N99554 | Hs.288959 | FLJ20920 | 17; 2 | 17q21.33 |
| 290760 | N99671 | Hs.5555 | MGC5347 | 15 | 15q15.1 |
| 124890 | R06111 | In multiple clusters | | | |
| 127410 | R08756 | Hs.311968 | | 6 | |
| 127462 | R08772 | Hs.512696 | FLJ12806 | 13; 1 | 1q42.12 |
| 128725 | R09977 | Hs.20495 | DKFZP434F011 | 6 | 6p25.2-p25.1 |
| 113527 | R10236 | Hs.503863 | FMNL2 | 2 | 2q24.1 |
| 128230 | R10430 | Hs.5978 | LMO7 | 13 | 13q21.33 |
| 129008 | R10465 | Hs.464896 | ZNF397 | 18 | 18q12.2 |
| 128490 | R10654 | Hs.221785 | | 19 | |
| 26053 | R11947 | Hs.354740 | KCNMA1 | 10 | 10q22-q23 |
| 26251 | R12819 | Hs.142395 | WDR5B | 3 | 3q21.1 |
| 27823 | R13127 | Hs.334045 | SKD3 | 11 | 11q13.3 |
| 26578 | R13806 | Hs.132037 | PES1 | 22 | 22q12.1 |
| 28684 | R14317 | Hs.244624 | | 2 | |
| 29330 | R14592 | | | | |
| 29702 | R15335 | Hs.411865 | IPO4 | 14 | 14q11.2 |
| 30905 | R18265 | | | | |
| 40473 | R18433 | Hs.4817 | OPCML | 11 | 11q25 |
| 31448 | R21456 | Hs.21945 | C6orf152 | 6 | 6q14.3 |
| 131880 | R24654 | Hs.271568 | | 20 | |
| 133919 | R28674 | | | | |
| 152936 | R50257 | Hs.142245 | HHLA3 | 1 | 1p31.2 |
| 43040 | R60440 | Hs.317466 | PTPN4 | 2 | 2q14.2 |
| 141606 | R69183 | Hs.189527 | RASGRP1 | 15; 19 | 15q15 |
| 155452 | R71966 | Hs.300052 | TCERG1 | 5 | 5q31 |
| 155443 | R72001 | Hs.321576 | TRIM3 | 11 | 11p15.5 |
| 156287 | R73484 | Hs.415312 | GLYCTK | 3 | 3p21.31 |
| 156718 | R73794 | Hs.147644 | ZNF331 | 19 | 19q13.3-q13.4 |
| 143221 | R73965 | Hs.76781 | ABCD3 | 1 | 1p22-p21 |
| 158873 | R75776 | Hs.128073 | CETN3 | 5 | 5q14.3 |
| 194489 | R86224 | Hs.84087 | KIAA0143 | 8 | 8q24.22 |
| 186803 | R88104 | Hs.458262 | IGL@ | 22 | 22q11.1-q11.2 |
| 195346 | R88887 | Hs.367690 | FLJ00005 | 15 | 15q23 |
| 195117 | R91258 | Hs.445447 | | 12 | |
| 195853 | R92310 | Hs.388438 | FLJ11457 | 2 | 2q31.1 |
| 196350 | R92545 | Hs.34590 | | 5 | |
| 195917 | R92625 | Hs.14039 | | 12 | |
| 198582 | R94810 | Hs.475896 | | 3 | |
| 200158 | R97892 | Hs.108002 | | 22 | |
| 206794 | R98282 | Hs.427202 | TTR | 11; 18; 22; 2 | 18q12.1 |
| 206857 | R98898 | Hs.27004 | | 9 | |
| 201458 | R99656 | Hs.459501 | | 15 | |
| 61411 | T39957 | Hs.142074 | | 2 | 2q12.1 |
| 72769 | T50835 | Hs.78061 | TCF21 | 6 | 6pter-qter |
| 30758 | T65459 | Hs.185140 | PIP3-E | 6 | 6q25.2 |
| 21496 | T65600 | Hs.111539 | | 20 | |
| 29402 | T66043 | In multiple clusters | | | |
| 42057 | T66070 | Hs.440905 | DDR2 | 10; 1 | 1q12-q23 |
| 53293 | T66756 | Hs.381912 | SPRY3 | X; Y | Xq28 and Yq12 |
| 110279 | T71508 | Hs.13861 | | 8 | |
| 112434 | T85994 | Hs.227750 | NDUFB4 | 3 | 3q13.33 |
| 118088 | T92243 | | | | |
| 118129 | T92269 | Hs.268012 | FACL3 | 2 | 2q34-q35 |
| 121117 | T96469 | Hs.447988 | MGC22014 | 2 | 2p13.1 |
| 121587 | T97837 | Hs.511664 | | 15 | |
| 123296 | T99946 | Hs.431001 | | 4 | |
| 278608 | W01339 | Hs.17240 | | 7 | |
| 294425 | W01543 | Hs.440835 | SF1 | 11; 17; 20; 22; 4 | 11q13 |
| 295599 | W02188 | Hs.271736 | | 2 | |
| 298259 | W03697 | Hs.362806 | GPR116 | 6 | 6p21.1 |
| 298551 | W04396 | Hs.443260 | C20orf20 | 20 | 20q13.33 |
| 300017 | W07144 | Hs.280226 | APOB | 2 | 2p24-p23 |
| 320288 | W31507 | Hs.353211 | CHRFAM7A | 15 | 15q13.1 |
| 321340 | W32160 | Hs.247734 | PCDHA5 | 11; 1; 5 | 5q31 |
| 327491 | W32727 | Hs.55410 | K6IRS3 | 12 | 12q13.3 |
| 327574 | W35163 | Hs.407934 | NAV2 | 11 | 11p15.1 |
| 322190 | W37882 | Hs.250112 | | 3 | 3p21.2 |
| 328591 | W39752 | Hs.73923 | PNLIPRP1 | 10 | 10q26.12 |
| 328721 | W40330 | Hs.2707 | GSPT1 | 16 | 16p13.1 |
| 324110 | W46660 | Hs.504998 | URKL1 | 20; 5 | 20q13.33 |
| 342181 | W61100 | Hs.79241 | BCL2 | 18 | 18q21.33 |
| 342523 | W68566 | Hs.274136 | BRF2 | 8 | 8p11.23 |
| 344490 | W72937 | Hs.443811 | CALD1 | 7 | 7q33 |
| 417363 | W88448 | Hs.16374 | | 14 | |
| 418152 | W90539 | Hs.175475 | ZNF490 | 19 | 19p13.2 |
| 415197 | W91959 | Hs.288801 | SSBP3 | 1; 6; 7 | 1p32.3 |
| 33839 | | | | | |
| 253147 | | | | | |
| 25099 | | | | | |
| 255277 | | | | | |
| 51773 | | | | | |
| 197626 | | | | | |
| 291525 | | | | | |
| 41775 | | | | | |
| 415250 | | | | | |
| 158318 | | | | | |
| 197755 | | | | | |
| 303192 | | | | | |
| 488934 | | | | | |
| 28147 | | | | | |
| 39518 | | | | | |
| 416095 | | | | | |
| 68500 | | | | | |
| 345143 | | | | | |
| 37097 | | | | | |
| 286965 | | | | | |
| 359898 | | | | | |
| 328256 | | | | | |
| 322571 | | | | | |
| 469898 | | | | | |
| 195107 | | | | | |
| 356962 | | | | | |
| 509663 | | | | | |
| 53385 | | | | | |
| 328316 | | | | | |
| 249708 | | | | | |
| 229645 | | | | | |
| 665393 | | | | | |
| 510197 | | | | | |
| 165957 | | | | | |
| 509460 | | | | | |
| 365515 | | | | | |
| 376533 | | | | | |
| 338612 | | | | | |
| 119013 | | | | | |
| 510706 | | | | | |
| 28896 | | | | | |
| 484533 | | | | | |
| 126230 | | | | | |
| 491371 | | | | | |
| 270348 | | | | | |
| 380591 | | | | | |
| 682892 | | | | | |
| 47391 | | | | | |
| 239886 | | | | | |
| 415787 | | | | | |
| 50077 | | | | | |
| 130949 | | | | | |
| 202537 | | | | | |
| 272871 | | | | | |

TABLE 3
2-Fold Downregulated EST's

| Original | Confirmed | Unigene | | | Chromosome |
|---|---|---|---|---|---|
| Clone ID | Clone ID | ID | Gene Name | Chromosome | location |
| 427856 | AA001321 | Hs.18160 | MYCT1 | 6 | 6q25.1 |
| 428198 | AA001803 | Hs.486684 | | 6 | |
| 428067 | AA002233 | Hs.172685 | XPO7 | 8 | 8p21 |
| 125342 | AA007669 | Hs.20010 | | 10 | |
| 430172 | AA010246 | Hs.17283 | FLJ10890 | 11 | 11p11.2 |
| 430283 | AA010427 | Hs.445619 | | 3 | |
| 429589 | AA011356 | Hs.159494 | BTK | X | Xq21.33-q22 |
| 429735 | AA011519 | Hs.26267 | TOR3A | 1 | 1q24.3 |
| 469296 | AA026200 | Hs.114777 | | 11 | 11q23.3 |
| 469177 | AA026651 | Hs.24192 | SYNPO2 | 4 | 4q27 |
| 469377 | AA026844 | Hs.135260 | C14orf44 | 14 | 14q24.2 |
| 469886 | AA028188 | Hs.467724 | | 2 | |
| 366887 | AA029596 | Hs.170195 | BMP7 | 20 | 20q13 |
| 470599 | AA031920 | Hs.68877 | CYBA | 16; 9 | 16q24 |
| 470576 | AA032010 | Hs.449009 | WARP | 1 | 1p36.33 |
| 429895 | AA033783 | Hs.271787 | | 1 | |
| 471639 | AA035031 | Hs.158969 | C2orf3 | 2 | 2p11.2-p11.1 |
| 484765 | AA037249 | Hs.155433 | ATP5C1 | 10 | 10q22-q23 |
| 484915 | AA037589 | Hs.439480 | RBM5 | 19; 3 | 3p21.3 |
| 484939 | AA037633 | Hs.191422 | | 2 | |
| 484960 | AA037640 | Hs.61884 | LOC148898 | 1 | 1p36.11 |
| 485753 | AA040112 | Hs.443120 | CD36 | 7 | 7q11.2 |
| 485912 | AA040134 | Hs.21276 | COL4A3BP | 10; 5 | 5q13.3 |
| 375920 | AA040295 | Hs.95231 | FHOD1 | 16 | 16q22 |
| 485953 | AA040631 | Hs.26703 | CNOT8 | 5 | 5q31-q33 |
| 486046 | AA040917 | Hs.61912 | | 10 | |
| 376279 | AA041245 | Hs.103417 | PLCE1 | 10 | 10q23 |
| 486809 | AA043296 | Hs.12845 | MGC13159 | 4 | 4p16.2 |
| 486544 | AA043334 | Hs.380092 | SNAPC3 | 9 | 9p22.2 |
| 486617 | AA043451 | Hs.74615 | PDGFRA | 4 | 4q11-q13 |
| 487487 | AA043491 | Hs.381300 | MGC57858 | 6 | 6p21.31 |
| 487341 | AA043717 | Hs.211202 | EDNRA | 4 | 4q31.22 |
| 486406 | AA043769 | Hs.370545 | | 8 | |
| 487317 | AA043800 | Hs.62645 | | 11 | |
| 487338 | AA043806 | Hs.405465 | ITGB3BP | 1 | 1p31.3 |
| 487071 | AA044049 | Hs.82963 | GNRH1 | 8 | 8p21-p11.2 |
| 376675 | AA045274 | Hs.11355 | TMPO | 10; 12; 6 | 12q22 |
| 489282 | AA045730 | Hs.82173 | TIEG | 8 | 8q22.2 |
| 488619 | AA045809 | Hs.396404 | SMARCA2 | 9 | 9p22.3 |
| 487372 | AA046481 | Hs.371458 | CLCN6 | 1 | 1p36 |
| 376867 | AA047000 | Hs.129959 | IL17RC | 3 | 3p25.3 |
| 488499 | AA047396 | Hs.198998 | CHUK | 10; 2; X | 10q24-q25 |
| 488458 | AA047506 | Hs.165216 | | 15 | |
| 489374 | AA054296 | Hs.108338 | DKFZp586C1924 | 11; 8 | 11q14.1 |
| 377111 | AA055054 | | | | |
| 377252 | AA055349 | Hs.45743 | ADORA2B | 17; 4 | 17p12-p11.2 |
| 488149 | AA057266 | Hs.301526 | TRIM45 | 1 | 1p12 |
| 489369 | AA058476 | Hs.77318 | PAFAH1B1 | 17; 2 | 17p13.3 |
| 488100 | AA058617 | Hs.481179 | | 4 | |
| 489594 | AA099522 | Hs.61763 | ZCWCC2 | X | Xq22.3 |
| 489742 | AA099730 | Hs.74050 | FVT1 | 18 | 18q21.3 |
| 490628 | AA101704 | Hs.7503 | FLJ14153 | 3 | 3q25.32 |
| 489600 | AA101906 | Hs.15780 | ABCA6 | 17 | 17q24.3 |
| 489702 | AA101980 | Hs.135052 | DKFZp686N19164 | 19 | 19q13.13 |
| 490607 | AA102836 | Hs.459133 | | 15 | |
| 491756 | AA115294 | Hs.77367 | CXCL9 | 4 | 4q21 |
| 502184 | AA128133 | Hs.22370 | nexilin | 1 | 1p31.1 |
| 502041 | AA128184 | Hs.319095 | | 15 | |
| 502106 | AA130381 | Hs.180257 | MGC41917 | 19 | 19q13.43 |
| 503692 | AA131618 | Hs.406678 | | 4 | |
| 490970 | AA136782 | Hs.61814 | | 15 | |
| 505273 | AA143168 | Hs.25035 | CLIC4 | 1 | 1p36.11 |
| 505351 | AA147249 | Hs.463302 | | | |
| 505479 | AA147581 | Hs.8769 | TM4SF10 | X | Xp11.4 |
| 491242 | AA148778 | Hs.118738 | VprBP | 3 | 3p21.31 |
| 503293 | AA149547 | Hs.183390 | FLJ13590 | 19 | 19q13.41 |
| 505054 | AA149847 | Hs.459987 | ANP32B | 15; 9 | 9q22.32 |
| 491268 | AA150341 | Hs.10846 | SAT2 | 17; 2 | 17p13.2 |
| 504649 | AA150632 | | | | |
| 505063 | AA150920 | Hs.443257 | C21orf108 | 21 | 21q22.11 |
| 505060 | AA151020 | Hs.83381 | GNG11 | 13; 7 | 7q31-q32 |
| 504927 | AA151092 | Hs.431099 | MAP17 | 1 | 1p33 |
| 503051 | AA151535 | Hs.448885 | | 17; X | 17q23.3 |
| 502253 | AA156809 | Hs.442709 | | 9 | |
| 502310 | AA156853 | Hs.181161 | KIAA1972 | 16 | 16q13 |
| 502516 | AA156925 | Hs.356079 | | 5 | |
| 502351 | AA156936 | Hs.350631 | AKAP13 | 15 | 15q24-q25 |
| 665672 | AA194200 | Hs.85908 | | 6 | |
| 665308 | AA195098 | Hs.504900 | | 12 | |
| 446456 | AA203190 | Hs.308022 | | 4 | |
| 446498 | AA203216 | Hs.365592 | | 5 | |
| 446795 | AA203380 | | | | |
| 446552 | AA203444 | Hs.446492 | COPS5 | 8 | 8q13.1 |
| 446779 | AA203463 | Hs.476164 | | 1 | |
| 446807 | AA203562 | | | | |
| 446196 | AA203735 | | | | |
| 469345 | AA227799 | Hs.61438 | | X | |
| 272580 | AA253204 | In multiple clusters | | | |
| 322797 | AA284268 | Hs.180178 | FLJ23749 | 8 | 8p23.1 |
| 298850 | AA393271 | Hs.54347 | LOC139231 | 17; X | Xq22.2 |
| 36627 | AA682925 | Hs.13245 | PRG1 | 1 | 1p21.3 |
| 113554 | AA707037 | Hs.443542 | | 19 | |
| 70206 | AI275977 | | | | |
| 147728 | AI368607 | Hs.442818 | FAM13A1 | 3; 4 | 4q22.1 |
| 141755 | AI819863 | Hs.106243 | | 18 | |
| 113707 | AI934407 | Hs.501565 | DHTKD1 | 10 | 10p14 |
| 486219 | AL079948 | | | | |
| 114786 | AL532234 | | | | |
| 503572 | AL533313 | | | | |
| 296232 | AL552613 | | | | |
| 501678 | AL566877 | | | | |
| 165952 | AL579734 | | | | |
| 31638 | AU139675 | | | | |
| 125698 | AV747676 | | | | |
| 127360 | AW296131 | Hs.193228 | AGXT2 | 5 | 5p13 |
| 487391 | AW371974 | | | | |
| 278730 | AW835275 | | | | |
| 470560 | AW949701 | | | | |
| 40183 | AW953295 | | | | |
| 114461 | BE003375 | | | | |
| 488478 | BE513417 | Hs.24178 | EML2 | 19 | 19q13.32 |
| 166049 | BE566623 | Hs.29899 | LOC285636 | 5 | 5p13.1 |
| 116177 | BF058993 | Hs.233240 | COL6A3 | 2 | 2q37 |
| 140444 | BG120386 | Hs.107149 | C1orf25 | 1 | 1q25.2 |
| 502320 | BG428958 | Hs.21330 | ABCB1 | 7 | 7q21.1 |
| 296779 | BG548742 | Hs.458462 | ALTE | X; Y | Xp22.33 |
| 471217 | BG548895 | Hs.82359 | TNFRSF6 | 10 | 10q24.1 |
| 114109 | BG571413 | Hs.502092 | PSG9 | 19 | 19q13.2 |
| 114041 | BG577269 | Hs.4096 | FLJ32452 | 12 | 12q13.13 |
| 446483 | BG616960 | Hs.2257 | VTN | 17 | 17q11 |
| 501492 | BG676028 | Hs.89626 | PTHLH | 12 | 12p12.1-p11.2 |
| 44258 | BG764810 | Hs.75432 | IMPDH2 | 3 | 3p21.2 |
| 40630 | BG900936 | | | | |
| 470892 | BG994719 | | | | |
| 34512 | BI755380 | Hs.380621 | EPI64 | 22 | 22q12.1-qter |
| 32837 | BM126848 | Hs.437072 | MGC50896 | 11 | 11q13.1 |
| 39903 | BM128395 | In multiple clusters | | | |
| 503601 | BM465343 | Hs.425427 | LYAR | 4 | 4p16.2 |
| 488104 | BM473860 | Hs.180141 | CFL2 | 14 | 14q12 |
| 162789 | BM479478 | Hs.33191 | KIAA1976 | 5 | 5q35.3 |
| 114982 | BM549305 | Hs.349121 | DDX17 | 22 | 22q13.1 |
| 116386 | BM552356 | Hs.211612 | SEC24A | 17; 5 | 5q31.2 |
| 23684 | BM687827 | | | | |
| 39589 | BM713585 | | | | |
| 32973 | BM718815 | | | | |
| 345954 | BM720154 | | | | |
| 289224 | BM724312 | | | | |
| 125109 | BM765259 | | | | |
| 240871 | BM782272 | | | | |
| 491221 | BM789783 | | | | |
| 298803 | BM793520 | | | | |
| 207215 | BM793706 | Hs.77961 | HLA-B | 10; 17; 2; 6 | 6p21.3 |
| 418004 | BM804755 | Hs.418271 | MEG3 | 14 | 14q32 |
| 109311 | BM903943 | Hs.171292 | EPRS | 1 | 1q41-q42 |
| 197485 | BM974232 | | | | |
| 665580 | BM976213 | | | | |
| 249614 | BM976477 | | | | |
| 136441 | BM978832 | | | | |
| 31055 | BM978991 | | | | |
| 134272 | BM980948 | | | | |
| 136255 | BM996267 | | | | |
| 273428 | BM998773 | Mm.24788 | 2310047M15Rik | 13 | 13 A3.3 |
| 490962 | BM999387 | Mm.92904 | AV016528 | 5 | 5 B1 |
| 253223 | BQ003252 | Hs.368950 | MEF2C | 5 | 5q14 |
| 276680 | BQ016937 | | | | |
| 306759 | BQ028891 | Hs.155376 | HBB | 11; 7 | 11p15.5 |
| 40300 | BQ052067 | Hs.20013 | P29 | 1 | 1p36.13-p35.1 |
| 375929 | BQ063621 | Hs.29665 | CLSTN1 | 16; 1; 3 | 1p36.22 |
| 504999 | BQ068637 | Hs.284464 | | 10; 7 | |
| 488797 | BQ071673 | Hs.32989 | RAMP1 | 2 | 2q36-q37.1 |
| 238991 | BQ073106 | Hs.398636 | HBA2 | 16; 3 | 16p13.3 |
| 43842 | F12220 | | | | |
| 150370 | H00846 | Hs.25274 | C11ORF4 | 11 | 11cen-q22.3 |
| 150735 | H02366 | Hs.527367 | | 7 | |
| 152226 | H03112 | Hs.28491 | SAT | X | Xp22.1 |
| 151676 | H03242 | Hs.409708 | | 14 | |
| 151526 | H03750 | Hs.386481 | | 3 | |
| 44095 | H06336 | Hs.13480 | | 8 | |
| 45049 | H08344 | Hs.106234 | | 21 | 21q22.3 |
| 45764 | H08476 | Hs.112049 | SBF1 | 22 | 22q13.33 |
| 46844 | H10129 | In multiple clusters | | | |
| 47297 | H10622 | Hs.302498 | RAB40B | 17 | 17q25.3 |
| 47799 | H11825 | Hs.22293 | | 1 | 1p36.22 |
| 49362 | H12153 | Hs.407520 | CHN2 | 7 | 7p15.3 |
| 48269 | H12154 | Hs.15744 | SH2B | 16 | 16p12.1 |
| 47204 | H12279 | In multiple clusters | | | |
| 148425 | H12367 | Hs.155376 | HBB | 11; 7 | 11p15.5 |
| 43847 | H12953 | Hs.433597 | ARHGAP15 | 2 | 2q22.3 |
| 163564 | H14060 | In multiple clusters | | | |
| 48660 | H14983 | Hs.507077 | | 12 | |
| 159403 | H15021 | Hs.190161 | LR8 | 2; 7 | 7q36.1 |
| 49218 | H15334 | Hs.81848 | RAD21 | 7; 8 | 8q24 |
| 32102 | H15482 | In multiple clusters | | | |
| 48471 | H15983 | Hs.173119 | DP1 | 1; 5 | 5q22-q23 |
| 47861 | H16463 | In multiple clusters | | | |
| 49144 | H16558 | Hs.21213 | MYO5A | 15 | 15q21 |
| 49252 | H16609 | Hs.286261 | FLJ20195 | 17; 5 | 5q31.3 |
| 50465 | H16916 | Hs.26479 | LSAMP | 3 | 3q13.2-q21 |
| 50475 | H17029 | Hs.434924 | | 1 | |
| 172087 | H18810 | Hs.316977 | IPO8 | 12 | 12p11.22 |
| 172774 | H19691 | Hs.282331 | SIRT5 | 6 | 6p23 |
| 172471 | H20256 | Hs.124142 | | 8 | |
| 172944 | H20386 | Hs.400801 | C12orf10 | 12; 17 | 12q13 |
| 172483 | H20435 | Hs.268788 | | 10 | |
| 51321 | H20640 | Hs.268016 | MRPS6 | 21 | 21q21.3-q22.1 |
| 160505 | H21970 | | | | |
| 173228 | H22652 | Hs.151413 | GMFB | 14 | 14q22.2 |
| 52052 | H23040 | In multiple clusters | | | |
| 51969 | H23114 | Hs.22870 | | 15 | |
| 52169 | H24253 | | | | |
| 161967 | H26216 | Hs.443518 | BPAG1 | 6 | 6p12-p11 |
| 161769 | H26454 | Hs.32234 | PPIL6 | 6 | 6q21 |
| 163170 | H27377 | Hs.151414 | DKFZp434O0515 | 2 | 2q31.3 |
| 162333 | H27657 | Hs.369441 | GIT1 | 17 | 17p11.2 |
| 186349 | H28750 | Hs.288773 | ZNF294 | 21 | 21q22.11 |
| 184156 | H30779 | Hs.198037 | KIAA0599 | 14 | 14q23.3 |
| 191637 | H38158 | Hs.271630 | | 18 | |
| 190644 | H38584 | In multiple clusters | | | |
| 175032 | H38790 | Hs.58785 | TMEM16D | 12 | 12q23.3 |
| 192289 | H39049 | Hs.155553 | CHST10 | 2; 7; 8 | 2q12.1 |
| 182411 | H42095 | Hs.326035 | EGR1 | 5 | 5q31.1 |
| 183281 | H43974 | Hs.32043 | | 2 | |
| 183177 | H44996 | Hs.431101 | | 1 | |
| 177814 | H46143 | Hs.435976 | BRUNOL4 | 18 | 18q12 |
| 193139 | H47397 | Hs.80720 | GAB1 | 4 | 4q31.1 |
| 193165 | H47409 | Hs.33922 | MGC9084 | 1 | 1q23.3 |
| 193277 | H47694 | Hs.310336 | | 19 | |
| 274353 | H49836 | Hs.441858 | | 2 | |
| 179068 | H50037 | Hs.407934 | NAV2 | 11 | 11p15.1 |
| 179357 | H50391 | Hs.130873 | ACP1 | 11; 2 | 2p25 |
| 179890 | H50910 | Hs.89499 | ALOX5 | 10; 22 | 10q11.2 |
| 203898 | H56545 | Hs.75360 | CPE | 4; 8 | 4q32.3 |
| 204465 | H58558 | Hs.291000 | DKFZp761G058 | 4 | 4q22.1 |
| 236390 | H61388 | Hs.408096 | FXR1 | 3 | 3q28 |
| 208463 | H62185 | Hs.8694 | L0C56965 | 15 | 15q22.33 |
| 238479 | H64555 | Hs.413843 | S100A2 | 1 | 1q21 |
| 233852 | H65941 | Hs.268887 | | 7 | |
| 212748 | H70085 | Hs.382199 | | 5 | 5q14.1 |
| 232692 | H73118 | In multiple clusters | | | |
| 214469 | H73833 | Hs.389898 | FNBP3 | 2 | 2q24.1 |
| 214832 | H74091 | In multiple clusters | | | |
| 29958 | H75354 | Hs.269257 | | 15 | |
| 239536 | H78273 | Hs.500367 | SPAG9 | 17 | 17q21.33 |
| 233592 | H78485 | Hs.385924 | MGC43690 | 6 | 6q27 |
| 230380 | H80354 | Hs.282050 | FLJ31265 | 3 | 3q22.1 |
| 230449 | H80397 | In multiple clusters | | | |
| 230501 | H81140 | Hs.303023 | TUBB1 | 20 | 20q13.32 |
| 240100 | H82674 | Hs.365365 | | 18 | |
| 223123 | H84131 | In multiple clusters | | | |
| 222711 | H84323 | Hs.12929 | HLC-8 | 17 | 17q25.1 |
| 220023 | H84591 | Hs.421377 | | 18 | |
| 223331 | H86636 | | | | |
| 252663 | H87934 | Hs.65425 | CALB1 | 8 | 8q21.3-q22.1 |
| 252858 | H88465 | Hs.129952 | AQR | 15 | 15q13.3 |
| 253143 | H89047 | Hs.154336 | | 9 | |
| 253245 | H89297 | Hs.416630 | SDS3 | 12 | 12q24.23 |
| 253544 | H89535 | Hs.241385 | IL1RAPL1 | X | Xp22.1-p21.3 |
| 253625 | H89878 | | | | |
| 142417 | N/A | | | | |
| 273411 | N/A | | | | |
| 266129 | N28870 | In multiple clusters | | | |
| 266520 | N31174 | Hs.102276 | FLJ12584 | 1; 2 | 2q37.1 |
| 265499 | N31254 | Hs.117865 | SLC17A5 | 6 | 6q14-q15 |
| 266838 | N31417 | Hs.450230 | IGFBP3 | 10; 7 | 7p13-p12 |
| 269957 | N40354 | Hs.15440 | | 10 | |
| 270908 | N42522 | Hs.174312 | TLR4 | 9 | 9q32-q33 |
| 271690 | N43822 | Hs.44690 | | 8 | |
| 273287 | N44976 | Hs.434957 | | 8 | |
| 277824 | N45479 | Hs.14945 | FACL6 | 5 | 5q31 |
| 273254 | N46054 | Hs.466377 | | 19 | |
| 273582 | N46270 | Hs.709 | DCK | 4 | 4q13.3-q21.1 |
| 277056 | N46727 | Hs.44979 | | 9 | |
| 277714 | N46885 | Hs.29680 | | 14 | |
| 279157 | N47159 | Hs.2998 | CNTN2 | 11; 1 | 1q32.1 |
| 276465 | N48424 | Hs.435947 | RBM15 | 1 | 1p13 |
| 276914 | N48558 | Hs.271730 | EP300 | 22 | 22q13.2 |
| 279619 | N49032 | Hs.283477 | CD99 | 2; X; Y | Xp22.32 |
| 281960 | N54209 | Hs.27021 | RIOK2 | 5 | 5q15 |
| 285769 | N69323 | Hs.323733 | GJB2 | 13 | 13q11-q12 |
| 287434 | N69787 | Hs.105642 | FLJ21125 | 22 | 22q11.21 |
| 284451 | N75104 | Hs.436350 | ZNF302 | 19; 20 | 19q13.12 |
| 254017 | N75196 | Hs.10715 | | 13 | |
| 244310 | N75713 | Hs.31297 | CYBRD1 | 2 | 2q31.1 |
| 287830 | N75919 | Hs.518521 | | 3 | |
| 244955 | N76256 | Hs.9884 | TUBGCP3 | 13 | 13q34 |
| 289676 | N77030 | Hs.409677 | MOBP | 3 | 3p21.33 |
| 289939 | N77149 | Hs.43913 | PIBF1 | 13 | 13q21.33 |
| 245413 | N77203 | Hs.35100 | | 17 | |
| 245462 | N77278 | In multiple clusters | | | |
| 287772 | N79343 | Hs.48499 | VPS54 | 2 | 2p13-p14 |
| 290017 | N80091 | In multiple clusters | | | |
| 254337 | N81181 | Hs.94210 | EYA1 | 8 | 8q13.3 |
| 293606 | N94120 | In multiple clusters | | | |
| 293637 | N94167 | Hs.132652 | | 4 | |
| 293751 | N94211 | Hs.430541 | SON | 21 | 21q22.1-q22.2 |
| 278736 | N98252 | Hs.440643 | | 19; 8 | |
| 123232 | R00205 | Hs.146957 | PRC | 10 | 10q24.32 |
| 123539 | R01464 | Hs.389415 | CACNA2D2 | 3 | 3p21.3 |
| 123926 | R01515 | Hs.83942 | CTSK | 19; 1 | 1q21 |
| 125280 | R05785 | Hs.194121 | | 9 | |
| 125294 | R05876 | Hs.75081 | APC | 5 | 5q21-q22 |
| 126327 | R06482 | | | | |
| 126402 | R06562 | | | | |
| 126414 | R06576 | Hs.113614 | ADD2 | 2 | 2p14-p13 |
| 126415 | R06581 | Hs.133130 | | 10 | |
| 126541 | R06810 | Hs.375119 | C21orf90 | 21 | 21q22.3 |
| 126674 | R06958 | Hs.482577 | | 5 | |
| 126675 | R06966 | Hs.12999 | C9orf37 | 9 | 9q34.3 |
| 126748 | R07083 | Hs.173220 | PB1 | 3 | 3p21 |
| 126713 | R07114 | Hs.271224 | PH-4 | 3 | 3p21.31 |
| 126766 | R07137 | Hs.109445 | HIC2 | 22 | 22q11.21 |
| 126829 | R07236 | Hs.6019 | DNAJC3 | 13 | 13q32 |
| 126848 | R07242 | Hs.424986 | | 1 | |
| 125697 | R07554 | In multiple clusters | | | |
| 125769 | R07679 | Hs.17949 | | 3 | |
| 125787 | R07689 | Hs.27262 | NDUFB2 | 7 | 7q34 |
| 125806 | R07738 | Hs.20028 | NDST3 | 4 | 4q27 |
| 127293 | R08418 | Hs.14799 | | 9 | |
| 127453 | R08682 | Hs.16537 | ZNF364 | 1 | 1q21.2 |
| 128775 | R10007 | Hs.194146 | | 2 | |
| 128889 | R10213 | Hs.431156 | PPP2R1B | 11 | 11q23.2 |
| 130047 | R11594 | Hs.19795 | | 1 | |
| 25402 | R11719 | Hs.15243 | NOL1 | 11; 12 | 12p13 |
| 25649 | R11920 | Hs.472868 | | 20 | |
| 29339 | R12883 | In multiple clusters | | | |
| 26583 | R14004 | Hs.412327 | SATB2 | 2 | 2q33 |
| 26611 | R14035 | In multiple clusters | | | |
| 28397 | R14205 | Hs.110457 | WHSC1 | 15; 1; 4 | 4p16.3 |
| 29555 | R15292 | Hs.14202 | ARHT1 | 17; 21; 8 | 17q12 |
| 53096 | R15789 | Hs.4992 | TSSC1 | 2 | 2p25.3 |
| 53236 | R15917 | Hs.142570 | | 3 | |
| 30315 | R16314 | Hs.20999 | SES3 | 2 | 2q31.1 |
| 30635 | R18166 | Hs.459514 | | | |
| 30498 | R18539 | Hs.511752 | MXD4 | 1; 4 | 4p16.3 |
| 30608 | R18610 | | | | |
| 27020 | R18784 | Hs.200016 | NUDT11 | X | Xp11.23 |
| 32983 | R18944 | Hs.22583 | SIN3A | 15 | 15q23 |
| 33196 | R18950 | Hs.12251 | LOC151963 | 3 | 3q29 |
| 33633 | R18967 | Hs.454533 | KIAA0930 | 12; 22 | 22q13.31 |
| 33475 | R18976 | Hs.22595 | FLJ10637 | 12 | 12p12.1 |
| 27262 | R19087 | Hs.343667 | ELOVL5 | 17; 6 | 6p21.1-p12.1 |
| 32993 | R19105 | Hs.283851 | | 7 | |
| 34379 | R19685 | Hs.64004 | | 5 | 5q14.3 |
| 34495 | R19715 | In multiple clusters | | | |
| 34949 | R19766 | Hs.459842 | | 16 | |
| 31625 | R19839 | Hs.162189 | TRAD | 3 | 3q21.2 |
| 34432 | R20173 | | | | |
| 32448 | R20215 | In multiple clusters | | | |
| 130243 | R22632 | Hs.263876 | FLJ11036 | 3 | 3p25.2 |
| 131380 | R23093 | Hs.148767 | RQCD1 | 2 | 2q35 |
| 34400 | R24553 | | | | |
| 35133 | R24735 | Hs.145431 | ATF7IP2 | 16 | 16p13.2 |
| 34315 | R25006 | Hs.79981 | GRM5 | 11 | 11q14.2 |
| 131830 | R25119 | In multiple clusters | | | |
| 36682 | R25310 | Hs.4268 | | 10 | |
| 36355 | R25701 | Hs.12346 | | 12 | |
| 132876 | R27500 | | | | |
| 133908 | R28095 | | | | |
| 133895 | R28525 | Hs.10784 | C6orf37 | 6 | 6q14 |
| 134003 | R30888 | Hs.443120 | CD36 | 7 | 7q11.2 |
| 134256 | R31161 | Hs.412253 | | 12 | |
| 134229 | R31965 | Hs.24321 | | 11; 16 | |
| 136006 | R34105 | Hs.1674 | GFPT1 | 2 | 2p13 |
| 37521 | R34737 | Hs.168241 | C14orf103 | 14; 15; 4 | 14q32.31 |
| 37464 | R35310 | Hs.362805 | MEIS2 | 15 | 15q13.3 |
| 38377 | R35343 | Hs.54347 | LOC139231 | 17; X | Xq22.2 |
| 37509 | R35539 | Hs.482730 | | 5 | |
| 33543 | R35619 | Hs.309684 | | 2 | |
| 136983 | R35752 | Hs.2465 | GPR105 | 3 | 3q21-q25 |
| 137298 | R36593 | Hs.343748 | FLJ20445 | 10 | 10q23.33 |
| 152429 | R46283 | Hs.327389 | | 15 | |
| 153283 | R50329 | Hs.111554 | ARL7 | 2 | 2q37.2 |
| 38733 | R50911 | Hs.388289 | KCNQ3 | 8 | 8q24 |
| 38748 | R51027 | Hs.153355 | QKI | 6 | 6q26-27 |
| 39529 | R52470 | Hs.4290 | | 5 | |
| 41551 | R52844 | Hs.380144 | | 2; 7; 9 | |
| 32852 | R53267 | In multiple clusters | | | |
| 40069 | R53680 | In multiple clusters | | | |
| 40175 | R53688 | Hs.173933 | NFIA | 1 | 1p31.3-p31.2 |
| 40022 | R54062 | Hs.171342 | CRNKL1 | 20 | 20p11.2 |
| 41825 | R54165 | Hs.125293 | LOC221002 | 10 | 10q11.21 |
| 41552 | R54194 | Hs.242947 | DGKI | 7 | 7q32.3-q33 |
| 39682 | R54282 | Hs.166563 | RFC1 | 4; 7 | 4p14-p13 |
| 154969 | R55448 | Hs.26213 | DNTTIP1 | 20 | 20q13.12 |
| 41486 | R59266 | Hs.22998 | NRXN1 | 2 | 2p21 |
| 42773 | R59802 | | | | |
| 42477 | R59887 | Hs.388715 | | 6 | |
| 41964 | R60395 | Hs.82201 | CSNK2A2 | 16; 18 | 16p13.3-p13.2 |
| 37743 | R60657 | Hs.138294 | | | |
| 42080 | R61018 | Hs.26815 | THAP10 | 15 | 15q22.32 |
| 42639 | R61092 | In multiple clusters | | | |
| 42660 | R61230 | In multiple clusters | | | |
| 139858 | R64240 | Hs.40719 | KIAA1164 | 15 | 15q21.3 |
| 139750 | R64647 | In multiple clusters | | | |
| 138271 | R65676 | Hs.28625 | | 8 | |
| 140455 | R65928 | Hs.154248 | ALS2CR3 | 2 | 2q33 |
| 140875 | R67239 | Hs.438778 | RBMS2 | 12 | 12q13.13 |
| 142565 | R70830 | Hs.29792 | | 17 | |
| 142573 | R70834 | Hs.12899 | FAF1 | 1 | 1p33 |
| 143000 | R71157 | Hs.15921 | FLJ10759 | 1 | 1p34.3 |
| 156269 | R72667 | In multiple clusters | | | |
| 156087 | R73175 | Hs.78788 | LZTR1 | 22 | 22q11.21 |
| 144221 | R76995 | Hs.155376 | HBB | 11; 7 | 11p15.5 |
| 146771 | R80441 | Hs.372288 | KIAA1238 | 12 | 12p13.31 |
| 146567 | R80870 | | | | |
| 196730 | R83560 | Hs.484789 | | 6 | |
| 275589 | R84745 | | | | |
| 197329 | R86677 | Hs.435302 | ZNF3 | 7 | 7q22.1 |
| 166024 | R87411 | Hs.226133 | GAS7 | 17; X | 17p |
| 166094 | R87552 | Hs.241431 | GNAO1 | 16 | 16q13 |
| 199139 | R87819 | Hs.19339 | | 10; 3 | |
| 195070 | R91227 | Hs.7179 | RAD1 | 5 | 5p13.2 |
| 196321 | R92536 | | | | |
| 198520 | R94856 | Hs.35381 | | 6 | |
| 199241 | R95866 | Hs.186544 | | 1 | |
| 199678 | R96720 | Hs.387183 | BLMH | 17 | 17q11.2 |
| 200260 | R96774 | Hs.308638 | CYP3A7 | 7 | 7q21-q22.1 |
| 200121 | R97845 | Hs.445120 | LAMA2 | 6 | 6q22-q23 |
| 32760 | T26460 | | | | |
| 73643 | T55782 | Hs.76894 | DCTD | 4 | 4q35.1 |
| 72273 | T58094 | Hs.344478 | FLJ32440 | 8 | 8q24.13 |
| 21538 | T65503 | In multiple clusters | | | |
| 109864 | T66795 | Hs.191117 | | 7 | |
| 67036 | T70401 | Hs.268581 | | 18 | |
| 110207 | T71398 | Hs.429695 | | X | |
| 22568 | T74308 | Hs.271980 | MAPK6 | 15 | 15q21 |
| 23596 | T77481 | Hs.3850 | NDEL1 | 17 | 17p13.1 |
| 108782 | T77698 | Hs.429511 | | 17 | |
| 24504 | T80481 | Hs.51649 | | 9 | |
| 109042 | T80685 | Hs.270050 | | 12 | |
| 127207 | T81682 | Hs.177894 | | 16 | |
| 113327 | T83911 | Hs.11881 | TM4SF4 | 3 | 3q25 |
| 111377 | T84468 | Hs.23643 | MST4 | X | Xq26.2 |
| 111839 | T84986 | Hs.270074 | | 18 | |
| 117105 | T87920 | Hs.51515 | | 4 | |
| 110060 | T89355 | Hs.104746 | EPB41L4A | 5 | 5q22.2 |
| 118235 | T92415 | Hs.178703 | LOC199675 | 19 | 19p13.3 |
| 118792 | T92527 | Hs.159087 | RAD23B | 9 | 9q31.2 |
| 118695 | T93186 | Hs.433151 | LOH3CR2A | 1; 3 | 3p24-26 |
| 117266 | T93721 | Hs.12797 | DHX16 | 13; 6 | 6p21.3 |
| 120042 | T94831 | Hs.102301 | TFPI | 2 | 2q31-q32.1 |
| 119435 | T94862 | Hs.188572 | | 2 | |
| 120036 | T94968 | Hs.443755 | | 10 | |
| 121306 | T96743 | Hs.471127 | | 2 | |
| 121326 | T96757 | Hs.194424 | | 2 | |
| 121389 | T96900 | Hs.13768 | CACNA2D4 | 12; 2 | 12p13.33 |
| 121142 | T97034 | Hs.408623 | KIAA0877 | 7 | 7p14.3-p14.2 |
| 121164 | T97049 | Hs.78146 | PECAM1 | 17 | 17q23 |
| 120288 | T97103 | Hs.17962 | | 1 | |
| 121938 | T97960 | | | | |
| 121715 | T98069 | | | | |
| 122329 | T99133 | Hs.301985 | | 19; 6 | |
| 122345 | T99191 | | | | |
| 122809 | T99689 | Hs.82283 | MTR | 1 | 1q43 |
| 47391 | W00433 | Hs.443012 | SEMA6A | 2; 5 | 5q23.1 |
| 278777 | W01758 | Hs.5085 | DPM1 | 20 | 20q13.13 |
| 291571 | W03390 | Hs.42151 | HNMT | 2 | 2q22.1 |
| 295446 | W04294 | In multiple clusters | | | |
| 295401 | W04472 | Hs.10724 | MRPS35 | 12 | 12p11 |
| 298365 | W04859 | Hs.402201 | | 8 | |
| 300096 | W07088 | Hs.293685 | | 11 | |
| 300683 | W07576 | Hs.18471 | WBSCR18 | 16; 7 | 7q11.23 |
| 300953 | W07809 | Hs.40608 | | 11 | |
| 300620 | W15151 | Hs.410378 | FKBP7 | 2 | 2q31.3 |
| 301768 | W16484 | Hs.77100 | GTF2E2 | 8 | 8p21-p12 |
| 301723 | W16557 | Hs.276770 | CDW52 | 1 | 1p36 |
| 302190 | W16724 | Hs.258855 | MLL | 11 | 11q23 |
| 301788 | W17098 | Hs.75636 | MYL7 | 7 | 7p21-p11.2 |
| 306462 | W20339 | Hs.3781 | LRRN3 | 7 | 7q31.1 |
| 307761 | W21187 | Hs.87491 | TYMS | 18 | 18p11.32 |
| 307308 | W21227 | Hs.333738 | BBS2 | 16 | 16q21 |
| 306860 | W23999 | Hs.14968 | PLAG1 | 6; 8 | 8q12 |
| 320392 | W31757 | Hs.372571 | MBNL2 | 13 | 13q32.2 |
| 327627 | W35189 | Hs.46743 | MKKS | 11; 20 | 20p12 |
| 305132 | W38744 | Hs.30148 | HIPK3 | 11; 5 | 11p13 |
| 323823 | W46196 | Hs.16537 | ZNF364 | 1 | 1q21.2 |
| 338457 | W51918 | Hs.2879 | CPA1 | 7 | 7q32 |
| 342038 | W60281 | Hs.12940 | ZHX1 | 8 | 8q24.13 |
| 342216 | W61167 | Hs.2839 | NDP | X | Xp11.4 |
| 344176 | W70006 | Hs.153752 | CDC25B | 20 | 20p13 |
| 344494 | W72939 | Hs.201253 | ch-TOG | 11 | 11p11.2 |
| 415669 | W78854 | Hs.193725 | PSMD5 | 9 | 9q34.11 |
| 415648 | W78914 | Hs.122591 | PILRA | 7 | 7q22.1 |
| 347414 | W81245 | Hs.75485 | OAT | 10 | 10q26 |
| 347751 | W81591 | Hs.1422 | FGR | 1 | 1p36.2-p36.1 |
| 347779 | W84339 | Hs.246310 | ATP5J | 17; 21 | 21q21.1 |
| 416019 | W85798 | Hs.22383 | FLJ11336 | 11 | 11p13 |
| 415961 | W85811 | Hs.430081 | | | |
| 416424 | W86923 | Hs.31323 | IKBKAP | 9 | 9q31 |
| 416856 | W87319 | Hs.438583 | FLJ10613 | X | Xp11.22 |
| 417140 | W87641 | Hs.375085 | | 3 | |
| 415691 | W88578 | In multiple clusters | | | |
| 417741 | W88715 | Hs.418271 | MEG3 | 14 | 14q32 |
| 418112 | W90197 | Hs.25155 | NET1 | 10 | 10p15 |
| 418206 | W90265 | In multiple clusters | | | |
| 418103 | W90498 | Hs.309316 | SSB | 2 | 2q31.1 |
| 418156 | W90540 | Hs.28102 | | 2 | |
| 418251 | W90777 | Hs.83795 | IRF2 | 4 | 4q34.1-q35.1 |
| 415212 | W91936 | Hs.442801 | DKFZP564B1162 | 1; 4 | 4q22.1 |
| 418402 | W92838 | Hs.58919 | | 18 | |
| 52521 | Z44365 | | | | |
| 504350 | | | | | |
| 469411 | | | | | |
| 665391 | | | | | |
| 134157 | | | | | |
| 127224 | | | | | |
| 415565 | | | | | |
| 491645 | | | | | |
| 126736 | | | | | |
| 127238 | | | | | |
| 119255 | | | | | |
| 166049 | | | | | |
| 127283 | | | | | |
| 429690 | | | | | |
| 121865 | | | | | |
| 150662 | | | | | |
| 485744 | | | | | |
| 488304 | | | | | |
| 161919 | | | | | |
| 31549 | | | | | |
| 627215 | | | | | |
| 238885 | | | | | |
| 126582 | | | | | |
| 193925 | | | | | |
| 279284 | | | | | |
| 683239 | | | | | |
| 41961 | | | | | |
| 278788 | | | | | |
| 359159 | | | | | |
| 328947 | | | | | |
| 116247 | | | | | |
| 118563 | | | | | |
| 357990 | | | | | |
| 359931 | | | | | |
| 142906 | | | | | |
| 347131 | | | | | |
| 363695 | | | | | |
| 366701 | | | | | |
| 72084 | | | | | |
| 487986 | | | | | |
| 195429 | | | | | |
| 357885 | | | | | |
| 505249 | | | | | |
| 273063 | | | | | |
| 347227 | | | | | |
| 328903 | | | | | |
| 253114 | | | | | |
| 682788 | | | | | |
| 114444 | | | | | |
| 147872 | | | | | |
| 126236 | | | | | |
| 377203 | | | | | |
| 35074 | | | | | |
| 28221 | | | | | |
| 502636 | | | | | |
| 125796 | | | | | |
| 488596 | | | | | |
| 50591 | | | | | |
| 198896 | | | | | |
| 469847 | | | | | |
| 187166 | | | | | |
| 501695 | | | | | |
| 665075 | | | | | |
| 175915 | | | | | |
| 109654 | | | | | |
| 429726 | | | | | |
| 490600 | | | | | |
| 39427 | | | | | |
| 121390 | | | | | |
| 176362 | | | | | |
| 485950 | | | | | |

TABLE 4
Significantly Different Genes EST's p < 0.01

| Original Clone | | | | | Chromosome | Seq ID |
|---|---|---|---|---|---|---|
| ID | Accession ID | Unigene ID | Gene Name | Chromosome | location | No |
| 26251 | R12819 | Hs.142395 | WDR5B | 3 | 3q21.1 | 1 |
| 26053 | R11947 | Hs.354740 | KCNMA1 | 10 | 10q22-q23 | 2 |
| 39296 | | | | | | 3 |
| 230449 | H80397 | | | | | 4 |
| 126236 | | | | | | 5 |
| 430088 | AA009864 | Hs.508253 | | 13 | | 6 |
| 489898 | AA114919 | Hs.74497 | NSEP1 | 13; 14; 15; 1; 7; 9 | 1p34 | 7 |
| 491591 | AA115138 | In multiple | | | | 8 |
| | | clusters | | | | |
| 502423 | AA135452 | Hs.444818 | CGGBP1 | 12; 3 | 3p12-p11.1 | 9 |
| 298850 | AA393271 | Hs.54347 | LOC139231 | 17; X | Xq22.2 | 10 |
| 381854 | AL522995 | | | | | 11 |
| 488040 | AW021004 | Hs.75639 | LOC91137 | 5; 8 | 5q22.2 | 12 |
| 140376 | AW955751 | | | | | 13 |
| 131764 | BG434320 | Hs.458318 | PSG4 | 19 | 19q13.2 | 14 |
| 281866 | BM755877 | | | | | 15 |
| 150082 | H01737 | Hs.499891 | | 10 | | 16 |
| 48670 | H15967 | Hs.79101 | CCNG1 | 5 | 5q32-q34 | 17 |
| 52539 | H23375 | Hs.13781 | MGC35097 | 3 | 3p21.31 | 18 |
| 204968 | H57391 | Hs.9994 | LIPC | 15 | 15q21-q23 | 19 |
| 222188 | H83491 | Hs.40507 | | 7 | | 20 |
| 252856 | H88463 | Hs.446676 | LYPLA1 | 6; 8 | 8q11.23 | 21 |
| 238711 | H89047 | Hs.154336 | | 9 | | 22 |
| 266982 | N31667 | In multiple | | | | 23 |
| | | clusters | | | | |
| 277492 | N47773 | | | | | 24 |
| 128921 | R10272 | Hs.20580 | SOAT2 | 12 | 12q13.13 | 25 |
| 40473 | R18433 | Hs.4817 | OPCML | 11 | 11q25 | 26 |
| 132876 | R27500 | | | | | 27 |
| 142664 | R70980 | Hs.75268 | SIAT4C | 11 | 11q23-q24 | 28 |
| 155406 | R71939 | Hs.172084 | PYGO2 | 1; X | 1q22 | 29 |
| 147050 | R80322 | Hs.196384 | PTGS2 | 1 | 1q25.2-q25.3 | 30 |
| 166094 | R87552 | Hs.241431 | GNAO1 | 16 | 16q13 | 31 |
| 21956 | T66211 | Hs.171391 | CTBP2 | 10; 5 | 10q26.2 | 32 |
| 110268 | T71491 | Hs.185084 | MSI2 | 17; 19 | 17q23.2 | 33 |
| 309830 | W23600 | Hs.62314 | | 10 | | 34 |
| 347414 | W81245 | Hs.75485 | OAT | 10 | 10q26 | 35 |
| 416053 | W85949 | Hs.500495 | | 17; 2 | 17q24.2 | 36 |
| 267803 | | | | | | |
| 491729 | | | | | | |
| 127810 | | | | | | |
| 150662 | | | | | | |
| 30405 | | | | | | |
| 121390 | | | | | | 37 |
| 193802 | | | | | | 38 |
| 501695 | | | | | | 39 |

TABLE 5
Significantly Different Genes
P < 0.001

| Original | Acces- | | | Chro- | | Seq |
|---|---|---|---|---|---|---|
| Clone | sion | Unigene | Gene | mo- | Chromosome | ID |
| ID | ID | ID | Name | some | location | No. |
| 26251 | R12819 | Hs.142395 | WDR5B | 3 | 3q21.1 | 1 |
| 26053 | R11947 | Hs.354740 | KCNMA1 | 10 | 10q22-q23 | 2 |
| 39296 | | | | | | 3 |
| 230449 | H80397 | | | | | 4 |
| 126236 | | | | | | 5 |

TABLE 6
Genes that form the basis of clustering to predict outcome

| Original | Confirmed | | | | Chromosome | |
|---|---|---|---|---|---|---|
| Clone ID | Clone ID | Unigene ID | Gene Name | Chromosome | location | Seq No |
| 430088 | AA009864 | Hs.271776 | | 13 | | 40 |
| 428641 | AA011705 | Unknown | UXS1 | 2 | 2q12.3 | 41 |
| 470184 | AA028975 | Hs.246970 | MAP4K5 | 14 | 14q11.2-q21 | 42 |
| 470117 | AA029861 | Hs.12272 | | | | 43 |
| 469944 | AA030025 | Unknown | | | | 44 |
| 470402 | AA031292 | Hs.25333 | IL1R2 | 2 | 2q12-q22 | 45 |
| 470567 | AA031836 | Hs.131714 | | 14 | | 46 |
| 470655 | AA032034 | Hs.296426 | | | | 47 |
| 471058 | AA034297 | Hs.177486 | APP | 15; 21 | 21q21.2 | 48 |
| 486409 | AA042836 | Hs.83795 | IRF2 | 4 | 4q34.1-q35.1 | 49 |
| 486169 | AA043607 | Hs.62601 | DKFZp434N2030 | 12 | 12q21.33 | 50 |
| 376799 | AA046886 | Hs.9096 | FLJ20473 | 3 | 3q21.2 | 51 |
| 488002 | AA054673 | Unknown | ZNF229 | 19 | 19q13.2 | 52 |
| 488018 | AA054744 | Hs.8379 | | 2 | | 53 |
| 488097 | AA058640 | Hs.130315 | KCNE4 | 2 | 2q36.3 | 54 |
| 488263 | AA088634 | Hs.75741 | ABP1 | 7 | 7q34-q36 | 55 |
| 490193 | AA116099 | Hs.4943 | MAGED2 | X | Xp11.2 | 56 |
| 502909 | AA128587 | Hs.301094 | | 16; 7 | | 57 |
| 504165 | AA130223 | Hs.12820 | USP39 | 2 | 2p11.2 | 58 |
| 491070 | AA136958 | Hs.26433 | FLJ21924 | 11 | 11p13 | 59 |
| 505292 | AA152025 | Hs.19545 | FZD4 | 11 | 11q14.2 | 60 |
| 376780 | AI668645 | Hs.356717 | MYL4 | 11; 17 | 17q21-qter | 61 |
| 258340 | AL040763 | Hs.146393 | | | | 62 |
| 257360 | AL558028 | Hs.90035 | | | | 63 |
| 263942 | AL575253 | Hs.183986 | | | | 64 |
| 293442 | AV751900 | Unknown | | | | 65 |
| 488040 | AW021004 | Hs.75639 | LOC91137 | 5; 8 | 5q22.2 | 66 |
| 294244 | BE833937 | Hs.3807 | | | | 67 |
| 114586 | BF083948 | Hs.11101 | | | | 68 |
| 278303 | BF194709 | Hs.12950 | | 2 | | 69 |
| 43740 | BF343687 | Hs.31588 | NOVA1 | 14 | 14q | 70 |
| 308160 | BF359869 | Hs.30732 | | | | 71 |
| 109410 | BF846191 | Hs.9645 | | | | 72 |
| 143452 | BF988344 | Unknown | | | | 73 |
| 259161 | BG122385 | Unknown | PRKAG2 | 7 | 7q35-q36 | 74 |
| 36809 | BG260688 | Hs.7750 | LOC64744 | 19; 1 | 1p35.3-p34.1 | 75 |
| 131764 | BG434320 | Hs.251850 | PSG4 | 19 | 19q13.2 | 76 |
| 271368 | BG527412 | Hs.155433 | ATPSC1 | 10 | 10q22-q23 | 77 |
| 267379 | BG717406 | Hs.129828 | | 12 | | 78 |
| 40159 | BG740145 | Hs.77910 | HMGCS1 | 5 | 5p14-p13 | 79 |
| 30595 | BG998589 | Hs.6107 | | | | 80 |
| 51366 | BI597712 | Hs.112237 | | 15 | | 81 |
| 32687 | BI914137 | Hs.343666 | PEPP3 | 1 | 1q32.1 | 82 |
| 264528 | BM313556 | Hs.7476 | | | | 83 |
| 183688 | BM455686 | Hs.324342 | FBXO33 | 14 | 14q13.3 | 84 |
| 138346 | BM562200 | Hs.86437 | PIK3AP1 | 10 | 10q24.2 | 85 |
| 249289 | BM687578 | Hs.215595 | | | | 86 |
| 113701 | BM694906 | Hs.112049 | | | | 87 |
| 249227 | BM717054 | Hs.18449 | | | | 88 |
| 262054 | BM801234 | Hs.154332 | EDEM1 | 3 | 3p26.1 | 89 |
| 152371 | BM916476 | Hs.75725 | TAGLN2 | 1; 8 | 1q21-q25 | 90 |
| 259220 | BM921829 | Hs.9527 | 38080 | 12; 2 | 2p23.3 | 91 |
| 121557 | BM924902 | Hs.13128 | ZNF205 | 14; 16 | 16p13.3 | 92 |
| 188272 | BM925337 | Hs.170238 | SCN1B | 19 | 19q13.1 | 93 |
| 265047 | BQ051520 | Hs.164256 | NPL4 | 17 | 17qter | 94 |
| 150129 | H04482 | Hs.163724 | | 6 | | 95 |
| 48262 | H12126 | Hs.170307 | | | | 96 |
| 148437 | H12419 | Hs.152818 | USP8 | 15; 6 | 15q15.3 | 97 |
| 159395 | H14932 | Hs.183 | | | | 98 |
| 49272 | H16646 | Hs.118666 | PP591 | 1 | 1q22 | 99 |
| 50416 | H17201 | Hs.21814 | IL20RA | 6 | 6q22.33-q23.1 | 100 |
| 50149 | H17703 | Hs.334775 | MGC20255 | 19; 6 | 19q13.31 | 101 |
| 50141 | H17788 | Hs.31066 | | X | | 102 |
| 172049 | H18883 | AMBIGUOUS | | | | 103 |
| 172504 | H21214 | Hs.211914 | NDUFS7 | 19 | 19p13.3 | 104 |
| 174878 | H21835 | Unknown | | | | 105 |
| 173587 | H22418 | AMBIGUOUS | SZF1 | 3 | 3p21 | 106 |
| 52539 | H23375 | Hs.13781 | MGC35097 | 3 | 3p21.31 | 107 |
| 52043 | H24142 | Hs.75893 | ANK3 | 10; 1 | 10q21 | 108 |
| 158110 | H26552 | AMBIGUOUS | MGC5395 | 11 | 11q12.2 | 109 |
| 52648 | H29772 | Hs.32501 | | 8 | | 110 |
| 190079 | H30637 | Hs.27362 | MRPS30 | 5 | Sq11 | 111 |
| 192729 | H38504 | Hs.78605 | SUMF2 | 7 | 7q11.1 | 112 |
| 175067 | H38797 | Hs.66170 | SMYD2 | 1 | 1q32.3 | 113 |
| 191858 | H40404 | Hs.101383 | | 2 | | 114 |
| 188132 | H43837 | Hs.256972 | FLJ16025 | 3 | 3q29 | 115 |
| 178533 | H46579 | Hs.141269 | | 1 | 1p13.3 | 116 |
| 179800 | H50885 | Hs.137732 | TRIM35 | 8 | 8p21.1 | 117 |
| 179848 | H50895 | Hs.239298 | MAP4 | 2; 3 | 3p21 | 118 |
| 203560 | H55982 | Hs.75655 | | | | 119 |
| 204526 | H58631 | AMBIGUOUS | | | | 120 |
| 208424 | H62935 | Hs.9688 | CMRF-35H | 17 | 17q25.2 | 121 |
| 206377 | H63586 | Hs.338207 | FRAP1 | 1 | 1p36.2 | 122 |
| 210782 | H66920 | Hs.283732 | FLJ10460 | 15; 18 | 15q14 | 123 |
| 210921 | H70961 | Hs.301183 | MAIL | 3 | 3p12-q12 | 124 |
| 233401 | H79911 | Hs.110099 | CBFA2T3 | 16 | 16q24 | 125 |
| 248977 | H82392 | Unknown | GUCY1B3 | 4 | 4q31.3-q33 | 126 |
| 256186 | H94541 | AMBIGUOUS | C20orf64 | 20 | | 127 |
| 242569 | H95467 | Hs.36353 | MIDORI | 15 | 15q25.2 | 128 |
| 249245 | N/A | N/A | | | | 129 |
| 281931 | N/A | N/A | | | | 130 |
| 268674 | N23942 | Hs.11039 | MEP50 | 1 | 1p13.2 | 131 |
| 263896 | N25434 | Hs.89404 | MSX2 | 5 | 5q34-q35 | 132 |
| 265701 | N28655 | Hs.75478 | ATP11B | 3 | 3q27 | 133 |
| 267778 | N34196 | Unknown | | 16 | | 134 |
| 270023 | N36043 | Hs.90375 | | | | 135 |
| 268239 | N36345 | Hs.159629 | MYO9B | 19 | 19p13.1 | 136 |
| 268853 | N36650 | Hs.43728 | GPX6 | 1 | 1p32 | 137 |
| 270086 | N40627 | Hs.171917 | KIAA1434 | 20 | 20p13 | 138 |
| 270515 | N41933 | Hs.5158 | KIAA0409 | 11 | 11p15.4 | 139 |
| 271741 | N43858 | Hs.42212 | | 2 | | 149 |
| 276621 | N43952 | Hs.236443 | SPTBN1 | 2 | 2p21 | 141 |
| 273062 | N44283 | AMBIGUOUS | | | | 142 |
| 273061 | N44287 | Hs.44754 | LOC55831 | 17; 3 | 3p25.3 | 143 |
| 281902 | N48163 | Hs.16034 | MGC13186 | 1 | 1q42.2 | 144 |
| 276462 | N48417 | AMBIGUOUS | GABPA | 21; 7 | 21q21-q22.1 | 145 |
| 282695 | N52737 | Hs.75297 | FGF1 | 5 | 5q31 | 146 |
| 281993 | N53337 | Hs.256398 | ADAM22 | 7 | 7q21 | 147 |
| 281844 | N54092 | Hs.152213 | WNT5A | 3 | 3p21-p14 | 148 |
| 283577 | N55030 | Hs.109276 | | | | 149 |
| 247145 | N59051 | Hs.198793 | MICAL2 | 11 | 11p15.3 | 150 |
| 277932 | N95702 | Hs.75850 | WASF1 | 6 | 6q21-q22 | 151 |
| 277927 | N95721 | AMBIGUOUS | | 2 | | 152 |
| 278650 | N99208 | Hs.356590 | PPP1R8 | 16; 1 | 1p35 | 153 |
| 123614 | R01499 | Hs.19002 | C20orf55 | 20; X | 20p13 | 154 |
| 128544 | R10227 | AMBIGUOUS | MRPL12 | 17 | 17q25 | 155 |
| 129403 | R11299 | Hs.2441 | DCL-1 | 2 | 2q24.2 | 156 |
| 129703 | R16944 | AMBIGUOUS | | 3 | | 157 |
| 31759 | R17802 | Hs.22302 | GTF3C4 | 9 | 9q34.3 | 158 |
| 32953 | R19493 | Hs.348872 | RIMS1 | 6 | 6q12-q13 | 159 |
| 33720 | R19554 | Hs.106440 | FLJ10156 | 17 | 17p13.2 | 160 |
| 35000 | R19995 | Hs.169161 | | | | 161 |
| 30884 | R24295 | Hs.23648 | LSM11 | 4; 5 | 5q33.3 | 162 |
| 36581 | R25307 | Hs.23838 | CACNA1D | 3 | 3p14.3 | 163 |
| 132550 | R25899 | Hs.303627 | HNRPD | 4 | 4q21.1-q21.2 | 164 |
| 132624 | R25977 | Hs.23294 | DKFZp667G2110 | 3 | 3q12.1 | 165 |
| 133326 | R27073 | Hs.24115 | | 13 | | 166 |
| 133386 | R27212 | Hs.238996 | DKFZP434K0427 | 12 | 12q24.11 | 167 |
| 134713 | R28285 | Hs.327078 | | | | 168 |
| 135641 | R32384 | Hs.77091 | DNASE1L1 | X | Xq28 | 169 |
| 153309 | R47867 | Hs.165636 | DIRAS2 | 9 | 9q22.31 | 170 |
| 39249 | R51773 | Hs.64096 | KIAA0427 | 18; 7 | 18q21.1 | 171 |
| 41548 | R52841 | Unknown | | | | 172 |
| 41670 | R52871 | Hs.20021 | VAMP1 | 12 | 12p | 173 |
| 41495 | R54119 | Hs.281348 | FLJ10895 | 10 | 10pter-q26.12 | 174 |
| 154461 | R54929 | Hs.301496 | ABCA9 | 17 | 17q24.2 | 175 |
| 154632 | R55131 | AMBIGUOUS | | 22 | | 176 |
| 41968 | R60838 | Unknown | | | | 177 |
| 42762 | R61160 | Hs.79307 | ARHGEF6 | X | Xq26 | 178 |
| 138533 | R63267 | Hs.28399 | | 5 | | 179 |
| 140791 | R66209 | Hs.194714 | SNAP29 | 22 | 22q11.21 | 180 |
| 34717 | R67169 | Hs.75238 | CHAF1B | 21 | 21q22.13 | 181 |
| 142562 | R70822 | Hs.171501 | USP11 | 2; 7; X | Xp11.23 | 182 |
| 142664 | R70980 | Hs.75268 | SIAT4C | 11 | 11q23-q24 | 183 |
| 155406 | R71939 | Hs.172084 | PYGO2 | 1; X | 1q22 | 184 |
| 156164 | R72784 | AMBIGUOUS | CARS | 11 | 11p15.5 | 185 |
| 143425 | R74572 | Hs.146668 | TDE2 | 6 | 6q22.32 | 186 |
| 143846 | R75977 | Hs.153595 | LRP2 | 2 | 2q24-q31 | 187 |
| 144659 | R76138 | Unknown | UCHL3 | 13 | 13q21.33 | 188 |
| 145073 | R77382 | AMBIGUOUS | FLJ10276 | 1 | 1p34.3 | 189 |
| 145539 | R78065 | Hs.226770 | DKFZp566C0424 | 1 | 1p36.13 | 190 |
| 146850 | R80719 | Hs.9280 | PSMB9 | 6 | 6p21.3 | 191 |
| 146939 | R81026 | Hs.160244 | MOB | 10 | 10q11.2 | 192 |
| 149105 | R82447 | AMBIGUOUS | SMURF1 | 7 | 7q21.1-q31.1 | 193 |
| 187156 | R83139 | Hs.153261 | | | | 194 |
| 187620 | R83613 | Hs.153227 | GAK | 4 | 4p16 | 195 |
| 194987 | R91005 | Unknown | | 5 | | 196 |
| 381842 | R93847 | Hs.85112 | IGF1 | 12 | 12q22-q23 | 197 |
| 198693 | T41346 | Unknown | | | | 198 |
| 21956 | T66211 | Hs.356286 | CTBP2 | 10; 5 | 10q26.2 | 199 |
| 109064 | T80698 | Hs.38085 | MGC15937 | 11 | 11q12.2 | 200 |
| 110756 | T83261 | Hs.14456 | NEDD1 | 12 | 12q23.1 | 201 |
| 112577 | T86030 | Hs.12621 | | 3 | | 202 |
| 121505 | T97503 | Hs.18075 | FLJ14675 | 9 | 9q22.33 | 203 |
| 121574 | T97820 | Unknown | PSME4 | 2 | 2p16.3 | 204 |
| 298597 | W04381 | AMBIGUOUS | MGC11349 | 3; 8 | 3q21.3 | 205 |
| 307665 | W21429 | Hs.57549 | DJ473B4 | X | Xq26.3 | 206 |
| 309829 | W23823 | Hs.75617 | COL4A2 | 13 | 13q34 | 207 |
| 310097 | W24228 | Hs.55158 | TTC8 | 14 | 14q31.3 | 208 |
| 310149 | W24360 | Hs.237868 | IL7R | 5 | 5p13 | 209 |
| 308548 | W24939 | Hs.274313 | IGFBP6 | 12 | 12q13 | 210 |
| 309583 | W30772 | Hs.82547 | RARRES1 | 3 | 3q25.32 | 211 |
| 309613 | W30787 | Hs.279884 | DNAJD1 | 13 | 13q14.1 | 212 |
| 321655 | W32940 | Hs.293902 | FLJ32115 | 12 | 12p13.1 | 213 |
| 321749 | W33066 | Hs.288936 | MRPL9 | 1 | | 214 |
| 327657 | W35195 | Hs.356779 | LLGL1 | 17; 22 | 17p11.2 | 215 |
| 327772 | W35269 | Hs.165175 | | 5 | | 216 |
| 321970 | W37279 | Hs.268231 | FUT11 | 10; 9 | 10q22.3 | 217 |
| 322035 | W37328 | Unknown | | 5 | | 218 |
| 322071 | W37640 | Hs.183887 | FLJ22104 | 11 | 11q14.1 | 219 |
| 415608 | W78830 | Hs.5212 | SMUG1 | 12 | 12q13.11-q13.3 | 220 |
| 417318 | W88921 | Hs.19872 | | 18 | | 221 |
| 417679 | W89099 | AMBIGUOUS | CYP4F12 | 19 | 19p13.1 | 222 |
| 418197 | W90363 | Unknown | | 2 | | 223 |
| 415294 | W92124 | Hs.104203 | MGC12981 | 2 | 2q21.2 | 224 |
| 38384 | Z43356 | Hs.101375 | | | | 225 |
| 38888 | Z43356 | Hs.101375 | | | | 226 |
| 682875 | Unresolved | Unresolved | | | | 227 |
| 249286 | Unresolved | Unresolved | | | | 228 |
| 265151 | Unresolved | Hs.77271 | | | | 229 |
| 258616 | Unresolved | Hs.90462 | | | | 230 |
| 135419 | Unresolved | Hs.24545 | | | | 231 |
| 321324 | Unresolved | Hs.289092 | | | | 232 |
| 491729 | No_seq | | | | | |
| 416037 | No_seq | | | | | |
| 682696 | No_seq | | | | | |
| 359630 | No_seq | | | | | |
| 488866 | No_seq | | | | | |
| 682980 | No_seq | | | | | |
| 291218 | No_seq | | | | | |
| 182422 | No_seq | | | | | |
| 469795 | No_seq | | | | | |
| 257442 | No_seq | | | | | |
| 73685 | No_seq | | | | | |
| 469754 | No_seq | | | | | |
| 124727 | No_seq | | | | | |
| 256880 | No_seq | | | | | |
| 299654 | No_seq | | | | | |
| 418427 | No_seq | | | | | |
| 279530 | No_seq | | | | | |
| 138505 | No_seq | | | | | |
| 154656 | No_seq | | | | | |
| 265210 | No_seq | | | | | |
| 267803 | No_seq | | | | | |
| 263733 | No_seq | | | | | |
| 140863 | No_seq | | | | | |

### REFERENCES

1. GOLDENBERG R L. The management of preterm labor. Obstet Gynecol 2002;100:1020-37.
2. HACK M, FANAROFF A A. Outcomes of children of extremely low birthweight and gestational age in the 1990's. Early Hum Dev 1999;53:193-218.
3. WARD R M, BEACHY J C. Neonatal complications following preterm birth. Bjog 2003; 110 Suppl 20:8-16.
4. MARTIN J A, HAMILTON B E, SUTTON P D, VENTURA S J, MENACKER F, MUNSON M L. Births: final data for 2002. Natl Vital Stat Rep 2003;52:1-113.
5. Agency for Healthcare Research and Quality. 2001 Nationwide Inpatient Sample: March of Dimes Perinatal Data Center, 2003.
6. ANANTH C V, MISRA D P, DEMISSIE K, SMULIAN J C. Rates of preterm delivery among Black women and White women in the United States over two decades: an age-period-cohort analysis. Am J Epidemiol 2001;154:657-65.
7. Health Canada. Canadian Perinatal Health Report, 2003. Ottawa: Minister of Public Works and Government Services Canada, 2003.
8. Births in Victoria 2001-2002. Perinatal Data Collection Unit: Department of Human Services, Victoria, 2003.
9. OLSEN P, LAARA E, RANTAKALLIO P, JARVELIN M R, SARPOLA A, HARTIKAINEN A L. Epidemiology of preterm delivery in two birth cohorts with an interval of 20 years. Am J Epidemiol 1995;142:1184-93.
10. PETROU S. Economic consequences of preterm birth and low birthweight. Bjog 2003;110 Suppl 20:17-23.
11. PETROU S, MEHTA Z, HOCKLEY C, COOK-MOZAFFARI P, HENDERSON J, GOLDACRE M. The impact of preterm birth on hospital inpatient admissions and costs during the first 5 years of life. Pediatrics 2003;112:1290-7.
12. LIPPER E G, ROSS G S, AULD P A, GLASSMAN M B. Survival and outcome of infants weighing less than 800 grams at birth. Am J Obstet Gynecol 1990;163:146-50.
13. KRAMER M S, DEMISSIE K, YANG H, PLATT R W, SAUVE R, LISTON R. The contribution of mild and moderate preterm birth to infant mortality. Fetal and Infant Health Study Group of the Canadian Perinatal Surveillance System. Jama 2000;284:843-9.
14. GONIK B, CREASY R K. Preterm labor: its diagnosis and management. Am J Obstet Gynecol 1986; 154:3-8.
15. IAMS J D, CREASY R K. Preterm labor and delivery. In: Creasy R K, Resnik R, eds. Maternal-Fetal Medicine. Philadelphia: Saunders, 2004.
16. COPPER R L, GOLDENBERG R L, DAVIS R O, et al. Warning symptoms, uterine contractions, and cervical examination findings in women at risk of preterm delivery. Am J Obstet Gynecol 1990; 162:748-54.
17. LAMS J D, JOHNSON F F, PARKER M. A prospective evaluation of the signs and symptoms of preterm labor. Obstet Gynecol 1994;84:227-30.
18. COLOMBO D F, IAMS J D. Cervical length and preterm labor. Clin Obstet Gynecol 2000;43:73545.
19. BARTNICKI J, CASAL D, KREADEN U S, SALING E, VETTER K. Fetal fibronectin in vaginal specimens predicts preterm delivery and very-low-birth-weight infants. Am J Obstet Gynecol 1996;174:971-4.
20. BENATTAR C, TAIEB J, FERNANDEZ H, LINDENDAUM A, FRYDMAN R, VILLE Y. Rapid fetal fibronectin swab-test in preterm labor patients treated by betamimetics. Eur J Obstet Gynecol Reprod Biol 1997;72:131-5.
21. COLEMAN M A, MCCOWAN L M, PATTISON N S, MITCHELL M. Fetal fibronectin detection in preterm labor: evaluation of a prototype bedside dipstick technique and cervical assessment. Am J Obstet Gynecol 1998;179:1553-8.
22. SENDEN I P, OWEN P. Comparison of cervical assessment, fetal fibronectin and fetal breathing in the diagnosis of preterm labour. Clin Exp Obstet Gynecol 1996;23:5-9.
23. LASHAY N, GILSON G, JOFFE G, QUALLS C, CURET L. Will cervicovaginal interleukin-6 combined with fetal fibronectin testing improve the prediction of preterm delivery? J Matem Fetal Med 2000;9:336-41.
24. GILES W, BISITS A, KNOX M, MADSEN G, SMITH R. The effect of fetal fibronectin testing on admissions to a tertiary maternal-fetal medicine unit and cost savings. Am J Obstet Gynecol 2000;182:43942.
25. LOPEZ R L, FRANCIS J A, GARITE T J, DUBYAK J M. Fetal fibronectin detection as a predictor of preterm birth in actual clinical practice. Am J Obstet Gynecol 2000;182:1103-6.
26. IAMS J D, CASAL D, MCGREGOR J A, et al. Fetal fibronectin improves the accuracy of diagnosis of preterm labor. Am J Obstet Gynecol 1995;173:141-5.
27. LEESON S C, MARESH M J, MARTINDALE E A, et al. Detection of fetal fibronectin as a predictor of preterm delivery in high risk asymptomatic pregnancies. Br J Obstet Gynaecol 1996; 103:48-53.
28. LUKES A S, THORP J M, JR., EUCKER B, PAHEL-SHORT L. Predictors of positivity for fetal fibronectin in patients with symptoms of preterm labor. Am J Obstet Gynecol 1997; 176:639-41.
29. MALAK T M, SIZMUR F, BELL S C, TAYLOR D J. Fetal fibronectin in cervicovaginal secretions as a predictor of preterm birth. Br J Obstet Gynaecol 1996;103:648-53.
30. MCKENNA D S, CHUNG K, IAMS J D. Effect of digital cervical examination on the expression of fetal fibronectin. J Reprod Med 1999;44:796-800.
31. PEACEMAN A M, ANDREWS W W, THORP J M, et al. Fetal fibronectin as a predictor of preterm birth in patients with symptoms: a multicenter trial. Am J Obstet Gynecol 1997;177:13-8.
32. HONEST H, BACHMANN L M, GUPTA J K, KLEIJNEN J, KHAN K S. Accuracy of cervicovaginal fetal fibronectin test in predicting risk of spontaneous preterm birth: systematic review. Bmj 2002;325:301.
33. LEE S K, MCMILLAN D D, OHLSSON A, et al. The benefit of preterm birth at tertiary care centers is related to gestational age. Am J Obstet Gynecol 2003;188:617-22.
34. FISK N M, CHAN J. The case for tocolysis in threatened preterm labour. Bjog 2003;110 Suppl 20:98-102.
35. GYETVAI K, HANNAH M E, HODNETT E D, OHLSSON A. Tocolytics for preterm labor: a systematic review. Obstet Gynecol 1999;94:869-77.
36. Effectiveness and safety of the oxytocin antagonist atosiban versus beta-adrenergic agonists in the treatment of preterm labour. The Worldwide Atosiban versus Beta-agonists Study Group. Bjog 2001;108:13342.
37. SAWDY R J, LYE S, FISK N M, BENNETT P R. A double-blind randomized study of fetal side effects during and after the short-term maternal administration of indomethacin, sulindac, and nimesulide for the treatment of preterm labor. Am J Obstet Gynecol 2003;188:1046-51.
38. NORTON M E, MERRILL J, COOPER B A, KULLER J A, CLYMAN R I. Neonatal complications after the administration of indomethacin for preterm labor. N Engl J Med 1993;329:1602-7.
39. CROWLEY P. Prophylactic corticosteroids for preterm birth. Cochrane Database Syst Rev 2000:CD000065.
40. CROWLEY P. Antenatal corticosteroids—current thinking. Bjog 2003;110 Suppl 20:77-8.
41. MACARTHUR B A, HOWIE R N, DEZOETE J A, ELKINS J. School progress and cognitive development of 6-year-old children whose mothers were treated antenatally with betamethasone. Pediatrics 1982;70:99-105.
42. DESSENS A B, HAAS H S, KOPPE J G. Twenty-year follow-up of antenatal corticosteroid treatment. Pediatrics 2000;105:E77.
43. HARDING J E, DALZIEL S R, WALKER N K, et al. Long term effects of antenatal exposure to betamethasone: thiry year follow-up of a randomised controlled trial. J Soc Gynecol Investig 2004;11:408.
44. SLOBODA D M, MOSS T J, GURRIN L C, NEWNHAM J P, CHALLIS J R. The effect of prenatal betamethasone administration on postnatal ovine hypothalamic-pituitary-adrenal function. J Endocrinol 2002;172:71-81.
45. BROCKLEHURST P, GATES S, MCKENZIE-MCHARG K, ALFIREVIC Z, CHAMBERLAIN G. Are we prescribing multiple courses of antenatal corticosteroids? A survey of practice in the UK. Br J Obstet Gynaecol 1999;106:977-9.
46. IKEGAMI M, JOBE A H, NEWNHAM J, POLK D H, WILLET K E, SLY P. Repetitive prenatal glucocorticoids improve lung function and decrease growth in preterm lambs. Am J Respir Crit Care Med 1997;156:178-84.
47. NEWNHAM J P, EVANS S F, GODFREY M, HUANG W, IKEGAMI M, JOBE A. Maternal, but not fetal, administration of corticosteroids restricts fetal growth. J Matern Fetal Med 1999;8:81-7.
48. HUANG W L, BEAZLEY L D, QUINLIVAN J A, EVANS S F, NEWNHAM J P, DUNLOP S A. Effect of corticosteroids on brain growth in fetal sheep. Obstet Gynecol 1999;94:213-8.
49. FRENCH NP, HAGAN R, EVANS S F, GODFREY M, NEWNHAM J P. Repeated antenatal corticosteroids: size at birth and subsequent development. Am J Obstet Gynecol 1999; 180:114-21.
50. FRENCH N P, HAGAN R, EVANS S F, MULLAN A, NEWNHAM J P. Repeated antenatal corticosteroids: effects on cerebral palsy and childhood behavior. Am J Obstet Gynecol 2004; 190:588-95.
51. MERCER B M, GOLDENBERG R L, DAS A, et al. The preterm prediction study: a clinical risk assessment system. Am J Obstet Gynecol 1996;174:1885-93; discussion 1893-5.
52. NORWITZ E R, ROBINSON J N. A systematic approach to the management of preterm labor. Semin Perinatol 2001;25:223-35.
53. COLTON T, KAYNE H L, ZHANG Y, HEEREN T. A metaanalysis of home uterine activity monitoring. Am J Obstet Gynecol 1995;173:1499-505.
54. MAULDIN J G, NEWMAN R B. Preterm birth risk assessment. Semin Perinatol 2001;25:215-22.
55. SONEK J D, IAMS J D, BLUMENFELD M, JOHNSON F, LANDON M, GABBE S. Measurement of cervical length in pregnancy: comparison between vaginal ultrasonography and digital examination. Obstet Gynecol 1990;76:172-5.
56. WATSON W J, STEVENS D, WELTER S, DAY D. Observations on the sonographic measurement of cervical length and the risk of premature birth. J Matem Fetal Med 1999;8:17-9.
57. HEINE R P, MCGREGOR J A, DULLIEN V K. Accuracy of salivary estriol testing compared to traditional risk factor assessment in predicting preterm birth. Am J Obstet Gynecol 1999;180:S214-8.
58. DOWD J, LAHAM N, RICE G, BRENNECKE S, PERMEZEL M. Elevated interleukin-8 concentrations in cervical secretions are associated with preterm labour. Gynecol Obstet Invest 2001;51:165-8.
59. LEITICH H, KAIDER A. Fetal fibronectin—how useful is it in the prediction of preterm birth? Bjog 2003;110 Suppl 20:66-70.
60. JOFFE G M, JACQUES D, BEMIS-HEYS R, BURTON R, SKRAM B, SHELBURNE P. Impact of the fetal fibronectin assay on admissions for preterm labor. Am J Obstet Gynecol 1999;180:581-6.
61. LOWE M P, ZIMMERMAN B, HANSEN W. Prospective randomized controlled trial of fetal fibronectin on preterm labor management in a tertiary care center. Am J Obstet Gynecol 2004; 190:358-62.
62. LYE S J, BERNSTEIN P, OSKAMP M. Is the attenuation of beta-adrenergic agonist efficacy during labor caused by elevated prostaglandin E levels? Am J Obstet Gynecol 1998;179:1168-74.
63. MICHEL M C, PINGSMANN A, NOHLEN M, SIEKMANN U, BRODDE O E. Decreased myometrial beta-adrenoceptors in women receiving beta 2-adrenergic tocolytic therapy: correlation with lymphocyte beta-adrenoceptors. Clin Pharmacol Ther 1989;45:1-8.
64. CASEY M L, MACDONALD P C. Biomolecular processes in the initiation of parturition: decidual activation. Clin Obstet Gynecol 1988;31:533-52.
65. CHALLIS J R G, MATTHEWS S G, GIBB W, LYE S J. Endocrine and paracrine regulation of birth at term and preterm. Endocr Rev 2000;21:514-50.
66. JOKHI P P, CHUMBLEY G, KING A, GARDNER L, LOKE Y W. Expression of the colony stimulating factor-1 receptor (c-fms product) by cells at the human uteroplacental interface. Lab Invest 1993;68:308-20.
67. APERLO C, SEVILLA L, GUERIN S, POGNONEC P, BOULUKOS K E. Synergistic effects of colony-stimulating factor 1 and leukemia inhibitory factor in inducing early myeloid cell differentiation. Cell Growth Differ 1998;9:929-37.
68. ALBERT DH, SNYDER F. Release of arachidonic acid from 1-alkyl-2-acyl-sn-glycero-3-phosphocholine, a precursor of platelet-activating factor, in rat alveolar macrophages. Biochim Biophys Acta 1984;796:92-101.
69. BAN C, BILLAH M M, TRUONG C T, JOHNSTON J M. Metabolism of platelet-activating factor (1-O-alkyl-2-acetyl-sn-glycero-3-phosphocholine) in human fetal membranes and decidua vera. Arch Biochem Biophys 1986;246:9-18.
70. MADSEN S A, CHANG L C, HICKEY M C, ROSA G J, COUSSENS P M, BURTON J L. Microarray analysis of gene expression in blood neutrophils of parturient cows. Physiol Genomics 2004; 16:212-21.
71. LYE S J, OU C-W, TEOH T-G, ERB G, STEVENS Y, CASPER R. The molecular basis of labour & tocolysis. Fetal and Maternal Medicine Review 1998;10:121-136.
72. CHALLIS J R, SLOBODA D M, ALFAIDY N, et al. Prostaglandins and mechanisms of preterm birth. Reproduction 2002;124:1-17.
73. MCKEOWN K J, CHALLIS J R, SMALL C, et al. Altered fetal pituitary-adrenal function in the ovine fetus treated with RU486 and meloxicam, an inhibitor of prostaglandin synthase-II. Biol Reprod 2000;63:1899-904.

What is claimed is:

1. A method for detecting one or more pre-term labor marker polypeptide or pre-term labor polynucleotides in a subject comprising:
(a) obtaining a sample from a subject;
(b) detecting in polypeptides or polynucleotides extracted from the sample one or more pre-term labor polypeptide or pre-term labor polynucleotide that are associated with pre-term labor; and
(c) comparing the detected amount with an amount detected for a standard.

2. A method of detecting pre-term labor in a subject, the method comprising comparing:
(a) levels of one or more pre-term labor polypeptides or pre-term labor polynucleotides markers that are extracted from a sample from the subject; and
(b) normal levels of expression of the markers in a control sample, wherein a significant difference in levels of markers, relative to the corresponding normal levels, is indicative of pre-term labor.

3. A method as claimed in claim 1 or 2 comprising:
(a) contacting a biological sample obtained from a subject with one or more binding agent that specifically binds to pre-term labor polypeptide markers or parts thereof; and
(b) detecting in the sample amounts of polypeptides that bind to the binding agents, relative to a predetermined standard or cut-off value, and therefrom determining the presence or absence of pre-term labor in the subject.

4. A method as claimed in claim 3 wherein the binding agent is an antibody.

5. A method for screening a subject for pre-term labor comprising (a) obtaining a biological sample from a subject; (b) detecting in polypeptides extracted from the sample the amount of one or more pre-term labor polypeptide markers; and (c) comparing the amount of markers detected to a predetermined standard, where detection of a level of markers different than that of a standard is indicative of pre-term labor.

6. A method as claimed in any preceding claim which further comprises detecting multiple pre-term labor polypeptide markers.

7. A method for determining the presence or absence of one or more pre-term labor marker in a subject comprising detecting one or more pre-term labor polynucleotide in a sample from the subject and relating the detected amount to the presence of pre-term labor.

8. A method as claimed in claim 7 wherein the polynucleotide detected is mRNA.

9. A method of claim 8 wherein the polynucleotide is detected by
(a) contacting the sample with oligonucleotides that hybridize to the polynucleotides; and
(b) detecting in the sample levels of nucleic acids that hybridize to the polynucleotides relative to a predetermined standard or cut-off value, and therefrom determining the presence or absence of pre-term labor in the subject.

10. A method as claimed in claim 9 wherein the mRNA is detected using an amplification reaction.

11. A method as claimed in claim 10 wherein the amplification reaction is a polymerase chain reaction employing oligonucleotide primers that hybridize to the polynucleotides, or complements of such polynucleotides.

12. A method as claimed in claim 9 wherein the mRNA is detected using a hybridization technique employing oligonucleotide probes that hybridize to the polynucleotides or complements thereof, wherein the mRNA is detected by (a) isolating mRNA from the sample and combining the mRNA with reagents to convert it to cDNA; (b) treating the converted cDNA with amplification reaction reagents and primers that hybridize to the polynucleotides, to produce amplification products; (d) analyzing the amplification products to detect an amount of mRNA encoding one or more markers; and (e) comparing the amount of mRNA to an amount detected against a panel of expected values for normal tissue derived using similar primers.

13. A method for diagnosing and monitoring pre-term labor in a subject comprising isolating polynucleotides in a sample from the subject; and detecting polynucleotides encoding pre-term labor polypeptide markers in the sample wherein the presence of higher or lower levels of polynucleotides encoding pre-term labor polypeptide markers in the sample compared to a standard or control is indicative of pre-term labor.

14. A method for monitoring the progression of pre-term labor in a subject, the method comprising: (a) detecting in a sample from the subject at a first time point, one or more pre-term labor polypeptide or polynucleotide markers; (b) repeating step (a) at a subsequent point in time; and (c) comparing levels detected in steps (a) and (b), and thereby monitoring the progression of pre-term labor.

15. A diagnostic composition comprising an agent that binds to a pre-term labor polypeptide marker or hybridizes to a polynucleotide encoding a pre-term labor polypeptide marker.

16. A method for assessing the potential efficacy of a test agent for preventing, inhibiting, or reducing pre-term labor in a subject, the method comprising comparing: (a) levels of one or more pre-term labor polypeptide or polynucleotide markers, in a first sample obtained from a subject and exposed to the test agent, and (b) levels of the markers in a second sample obtained from the subject, wherein the sample is not exposed to the test agent, wherein a significant difference in the levels of expression of the markers in the first sample, relative to the second sample, is an indication that the test agent is potentially efficacious for preventing, inhibiting or reducing pre-term labor in the subject.

17. A method of assessing the efficacy of a therapy for preventing, inhibiting, or reducing pre-term labor in a subject, the method comprising comparing: (a) levels of one or more pre-term labor polypeptide or polynucleotide markers in a first sample obtained from the subject; and (b) levels of the markers in a second sample obtained from the subject following therapy, wherein a significant difference in the levels of expression of the markers in the second sample, relative to the first sample, is an indication that the therapy is efficacious for preventing, inhibiting, or reducing pre-term labor in the subject.

18. A method of selecting an agent for preventing, inhibiting or reducing pre-term labor in a subject the method comprising (a) obtaining a sample containing one or more pre-term labor polypeptide or polynucleotides from the subject; (b) separately exposing aliquots of the sample in the presence of a plurality of test agents; (c) comparing levels of one or more pre-term labor polypeptide or polynucleotide markers in each of the aliquots; and (d) selecting one of the test agents which alters the levels of markers in the aliquot containing that test agent, relative to other test agents.

19. A method of preventing, inhibiting, or reducing pre-term labor in a subject, the method comprising (a) obtaining a sample containing one or more pre-term labor polypeptide or polynucleotides from the subject; (b) separately maintaining aliquots of the sample in the presence of a plurality of test agents; (c) comparing levels of one or more pre-term labor polypeptide or polynucleotide markers in each of the aliquots; and (d) administering to the subject at least one of the test agents which alters the levels of markers in the aliquot containing that test agent, relative to other test agents.

20. A method of assessing the potential of a test compound to cause pre-term labor, the method comprising: (a) maintaining separate aliquots of samples containing one or more pre-term labor polypeptide or polynucleotides in the presence and absence of the test compound; and (b) comparing expression of one or more pre-term labor polypeptide or polynucleotide markers, in each of the aliquots, and wherein a significant difference in levels of markers in the aliquot maintained in the presence of the test compound, relative to the aliquot maintained in the absence of the test compound, is an indication that the test compound potentially causes pre-term labor.

21. A method of any preceding claim wherein the markers are one or more of the polynucleotides or polypeptides encoded by the polynucleotides in Table 2, 3, 4, 5 and/or 6, or SEQ ID Nos. 1 through 232.

22. A method of any preceding claim wherein the sample is maternal peripheral blood cells, more particularly mononuclear leukocytes.


SEQUENCE LISTING (232 sequences)

SEQ ID NO: 1 - DNA, 200 bp, Homo sapiens
gcagtgcacacctgctggtacgagcaaaagtagccgacaggccagagtttccagtttcggtggcttcgtaggcttttagcttctgaaatccgctaaaagcggcggttcattcatcttcaagtacctagcctgactggtagggctgctcagcattccgaaacatttcgacctggggaggggggcggcggtgaaagaatact

SEQ ID NO: 2 - DNA, 502 bp, Homo sapiens
  misc_feature (58)..(58): n is a, c, g, or t
gattgcgaagcatttgaaaagctttcacgatttgtgtagatgattatgaaggacctgnttgntgcaagagaacatcagtgatttttttagttactcaccaaggccttttgtcccagagccagttccctctgggagttcttatgaacatttctcaccttaatatggaggagagaatagtattccaatcatggatgtatcaaattctagtcatttagtttaagtgaaaagaggtttgattgcatattaaattgttattctgtctccttatgttgccatatgaatagctattttttttctttcacttttgacatttgggatganaagccatatgtatcataaatatcagatgtaagtcattaaaaactgccttcctgggacttttacatcttttaaaaggtgaattacttaccttatgtacagaataaataatgctcaggaaagagcaagtatttttccatgcattctcaggggatcttttactcccctttgnttgatanttagg

SEQ ID NO: 3 - DNA, 667 bp, Homo sapiens
gcggccgcaggattttttttttttttttttgttagttaaatagtttaatcagagcggttttacagttctaaaattaacaagcagacttgtaataaaagagctccaatgtttgcaaaatgaccttgtttccattctgaatctgactttttaattgtaaaggttagcattacatttaaaaggactcaaaatggaaaaaatggagtgatgtatattatcatccgtgttttgtgacgcaactgatgaaatccctgaatggatattgcttcagctttgatattaaaaaatggcctgacaagaagtccttaatcaacaaaactgatcacaggtggctgaaagaatgagcaaaacccaaaactactgtcaagttcatattgtatcacattgttggtggatgacactggctccagatatacaattaagcccacgacactttattactatgaataattatcatgaaatttctctcacgttgatcaacttgtttctgaaggttttctttgttcatttaaacttgcataataaacatgcaatgaatggtttcttttgaaagtgtattttttgcagtctttttgttttcatacaaatatttcataaagttgcaatgttgcatcagagcattaccaaaataatagcaggagcatatgggggcgggggcggcggggggggg

SEQ ID NO: 4 - DNA, 83 bp, Homo sapiens
aaccgaaattcagtcttttcttcaactgtgaatgtaggcagcaagccacagtggaacttgcagaacctataaatttgtcactg

SEQ ID NO: 5 - DNA, 667 bp, Homo sapiens
cctcgaggccagaattcggcacgagccccattttaagacaggaaaatgggctcagagaaggatcaacttgcctgagcacaacagggccacagcctgtctcccagcccccagatcaggccattgaccaagctgaggatgcagagctgggatccttgccctttgttctgtgttgtcccctggatgtgatggttccttggaactgaagagggcctggactcagtggcaggcatacactccaggccccagtgccatcagtcacacacaggagctgggttagggacggggctttactctctctcgagtgagtgtctccagtaggctatgccatgaggggagggtctgttagaaccaagatgaggaagaaagaaagggagagagagcagttccaagccagagttgttgaacaacaaggccaaggttgcagaatgaggcaggggtgaatctgagaccagaacccaggctccaggttccaggcttccacgtgagatgcgcaaatatttgagaatcttgcactgtacaggctccaggctgtatttgtccccagccagcttcaacttgtctatctgatgggctgagtgtctatatgagtgtccttggcagtgatgacctagtggactgcccagccccagaagagctggctgtcacctgtgtgccaggatcccagtgg

SEQ ID NO: 6 - DNA, 470 bp, Homo sapiens
  misc_feature (466)..(466): n is a, c, g, or t
ccagtatacatttattagactctacatgcattttagaatcagcttgtcgattttgatagagaagtcagttatgattctggtagggattacattgaatctgtagattacatatgttacaattaagtaatctacattgatgtattatcacccaaaataaatagtgtacattaggattcactcttggtgttggcttttcatagcttttagacaaattcacaatgatacataaccactaatacactatcatacagaggtttttttttactgccttaaatcccctgtgctcttcctgttgatctctccctctccctaatcctggcaaccactactccttttacatttatagtcataattgctaaaagtttagaaacaaccaagatgttcttcagtaggtaaatggatgaagaaactgtggtgccttgagacaatggaatgttattaccactaaaagaaaaaaaaaaaaatnttta

SEQ ID NO: 7 - DNA, 466 bp, Homo sapiens
  misc_feature (438)..(438): n is a, c, g, or t
gcggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacataaagacgtctctttttggtaataacaaacgtgttttttaaaaaagnctggtttttctcaatacgcctttaaagg

SEQ ID NO: 8 - DNA, 275 bp, Homo sapiens
ggtaccgggctctgtgagctagatcggttgcttcaggaacttaatgccactcagttcaacatcacagatgaaatcatgtctcagttcccatctagcaaggtggcttcaggagagcagaaggaggaccagtctgaagataagaaaagacccagcctcccttccagcccgtctcctggcctcccaaaggcttctgccacctcagccactctggagctggatagactgatggcctcactctctgacttccgcgttcaaaaccatcttccagcctctgg

SEQ ID NO: 9 - DNA, 536 bp, Homo sapiens
  misc_feature (532)..(532): n is a, c, g, or t
gttttttttgcctgcaactatatacacattgcaaaactattctgcgtcacatgattttaaatgaaataaatacaaaaatgaaccacacaatcaacacataactttaatactccacatttatcttgatcacaatggtggtaacctcctagtcaacaatcttgtgagttgaggagttgattctcattctcatatccatcaggaagatatgccctccgtagctggtctgacttaggtatggagcctccattcttcacatggcgagataggaaagcacggactgctgggtgatcagccttctcaagtgggatgttggcttccaggcacattttcacaaagtcctggataacactgactttctctgtttgcgcagtactgttgcactgaagagatgcagttaggggcctctgcttctttctcacattctgctcttcaaattctgccttcctcttggtatgagtctttgacttgaggtggtcactaatggcagacttgcgaacatgattcagaacccattgcaagaagtgcagagaantttc

SEQ ID NO: 10 - DNA, 482 bp, Homo sapiens
  misc_feature (459)..(459): n is a, c, g, or t
gtataatggcaaattaaattttaatttgacactcataccgaactcagaaaagcttgtttgatcttctctaaattctgtatgtagtactttttttaattgtcacatttctttgttacatctttgtgtccaacaaaacctgtggaaatctgtgttcatttttgtggtgagggatgggggtgaaaaaggggagacttgctctaattatgcatatacatttgactatgtaaaggtagttgtagactatatgtataaataacaagttaacacttaatattctaaattgttctaaggaacagtggcctgaagccatgactagttttgtggagctgctggctcttttaacagtagctgacctattggatttaaactgagactgaaattgacatttgtggttaggttatctagctgaggcttttaacaatggagcagataatgtctgttcctgtagtggatccattnaatttcctatttgagtactctcc

SEQ ID NO: 11 - DNA, 517 bp, Homo sapiens
  misc_feature (514)..(514): n is a, c, g, or t
gccaatagcaaaaatgatcgaaggaaccggaagtttaaggaagctgagcggctcttcagtaaatcctcggttacctcagcagctgcagtaagtgcattggcaggagttcaggaccagctcattgaaaagagggagccaggaagtgggacggaaagtgacacttctccagacttccacaatcaggaaaatgagcccagccaggaggaccctgaggatctggatggatctgtgcagggagtgaaacctcagaaggctgcttcttctacttcctcagggagtcaccacagcagccataaaaagcgaaagaataaaaaccggcacagcccgtctggcatgtttgattatgactttgagattgatctgaagttaaacaaaaaaccacgagctgactattagaagacacattagtgcagaagcttccaggctgtagagccctgcttcccttctctgacctcacaaagataaacatccttcacctgagttcgtggccatccacctctgctctcccagaccngtg

SEQ ID NO: 12 - DNA, 431 bp, Homo sapiens
  misc_feature (16)..(16): n is a, c, g, or t
ggtcacttgaaaatgngtatttaataccttagctggtgttttcagaagcatacagaactggaaaggtaagaacttgcttactattgcagatatacctgaccactcacaactgtctttttagcagcagctttgttaaaactgcacaaagcattcactttattgattgagtacaaatgctttagtgtttctacctaagtattagtacatctgttcaggaaatggaaaaaaaaaatgctgcttaaatttgttttatcagctgaggttgtggaggatttttgtccactgatcttttgcaacagaaaagcatactttgtcagcttctaataagatagccaggtttgaattatttgcaaaatttatataagcaaataagatctcttggtaacaattgacattgcactanaatttgaatgatactgttgttggcattt

SEQ ID NO: 13 - DNA, 444 bp, Homo sapiens
gcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaatgtcatgattgccgggggtgaaggcaagagatgaattgcattatttatatattttttattaaaatt

SEQ ID NO: 14 - DNA, 324 bp, Homo sapiens
gggagaaaacctctacttgtcctgcttcgcggaatctaacccaccggcagagtatttttggacaattaatgggaagtttcagcaatcaggacaaaagctctctatcccccaaattactacaaagcatagagggctctatacttgctctgttcgtaactcagccactggcaaggaaagctccaaatccatgacagtcgaagtctctgctccttcaggaataggacgtcttcctctccttaatccaatatagcagccgtgaagtcatttctgtatttcaggaagactggcagacagttgctttgattcttcctcaaactacttaca

SEQ ID NO: 15 - DNA, 417 bp, Homo sapiens
  misc_feature (393)..(393): n is a, c, g, or t
gtaaccttttcctaaagattgaccaaacagcaactagtagttatagaaaatctactcatttgtagatacagagaaaaatgaagaagatggaaaaagactcaaaagaggctttttaagttattcttcaaagcacttttcacatttccccataccctttctcacaaaaaaagtgtcataattaagtaatggtattgtttactgtttaaaagttaaaagatcaaaaatttgcttttatcccagtttttaaccacaaaaaaaagcgtagggattatccatgaggacttcatgccaagcaagaacctcaaacaaactagacaaactttttttttgacagtgaatgactttttgtaggacctgtgcgtgcgaaacccatggcaattgtcacatcctctnggtatgctggcagattgcttctct

SEQ ID NO: 16 - DNA, 494 bp, Homo sapiens
  misc_feature (393)..(393): n is a, c, g, or t
gccatgttggccagactggtctcaaactcctgacctcaggtgatccgcccacttcggcctcccaaagtgctgggattacaggcataaaccaccaggcccagcctacttgacccattgttaaccagcttgcttaaccaatcttggctagaaagaccttttttcccctgtgaaggcaactttgttagatgattatcagtttcacaatgagaagacccagatgaaggaggagcctcaatactgattgatttcctaaaacatgttcctaaaacagcccagaccaaagaacccaagtataactgaagattccaacctggacatggagcaactgcaacctatcaattactactggaagcacaggaaacctttattcctaatgagagaaaattctcttangaaaaaggcccatagatacatttctaaaaacaagagttttcttaagacttttccaaagcagaattctttgaagaaaaaaagaaaagcatcccgatcagatc

SEQ ID NO: 17 - DNA, 414 bp, Homo sapiens
  misc_feature (363)..(363): n is a, c, g, or t
taaagcaaagccttctgtgttggcattgtctatcattgcattagagatccaagcacagaagtgtgtagagttaacagaaggaatagaatgtcttcagaaacattccaagataaatggcagagatctgaccttctggcaagagcttgtatccaaatgtttaactgaatattcatcaaataagtgttccaaaccaaatgttcagaagttgaaatggattgtttctgggcgtactgcacggcaattgaagcatagctactacagaataactcaccttccaacaattcctgaaatggtcccttaactggattattacagcaccaaaaaacttctctgaagcctttctccacaaccttgttctatggnattccataatgttacaatggatttaagctatgaagcctcaaaacatcacga

SEQ ID NO: 18 - DNA, 551 bp, Homo sapiens
  misc_feature (549)..(549): n is a, c, g, or t
gcctcgtgccagcggaacggcggaccccgtaccctggcagcatcggagcaccggcggatggtatggtgtatgctctggggggaatgggccctgacacggccccccaggcccaggtacgtgtgtatgagccccgtcgggactgctggctttcgctaccctccatgcccacaccctgctatggggcctccaccttcctgcacgggaacaagatctatgtcctggggggccgccagggcaagctcccggtgactgcttttgaagcctttgatctggaggcccgtacatggacccggcatccaagcctacccagccgtcgggcctttgctggctgcgccatggctgaaggcagcgtctttagcctgggtggcctgcagcagcctgggccccacaacttctactctcgcccacactttgtcaacactgtggagatgtttgacctggagcatgggtcctggaccaaattgccccgcagcctgcgcatgagggataagagggcagactttgtggttggggtccttgggggccacattgtggccattgggggcttgnaa

SEQ ID NO: 19 - DNA, 488 bp, Homo sapiens
  misc_feature (443)..(443): n is a, c, g, or t
agcctgggtgcacacgtgtcaggatttgccggcagttccatcggtggaacgcacaagattgggagaatcacagggctggatgccgcgggacctttgtttgagggaagtgcccccagcaatcgtctttctccagatgatgccagttttgtggatgccattcatacctttacccgggagcacatgggcctgagcgtgggcatcaaacagcccataggacactatgacttctatcccaacgggggctccttccagcctggctgccacttcctagagctctacagacatattgcccagcacggcttcaatgccatcacccagaccataaaatgctcccacgagcgatcggtgcaccttttcatcgactccttgctgcacgccggcacgcagagcatggcctacccgtgtggtgacatgaacagcttcagccagggcctgtgcctganctgcagaaaggccgctgcaacacgctgggctaccacgtcgccagg

SEQ ID NO: 20 - DNA, 254 bp, Homo sapiens
  misc_feature (252)..(252): n is a, c, g, or t
gcctttactaacatttattttgattttaaggtaatttttatattataggttacttgctgactttttttcttctctgttcttttctgaagttgcattcttgtgttgggtgggacacagttaagtgtgacatgaatttccttgccccaagttcttgatttgcccatgaaatgaagacttgtgcctcatcagatgtcttttaaattttttactctgcacattttattttaattatctctgaaggcattttctggntc

SEQ ID NO: 21 - DNA, 200 bp, Homo sapiens
gaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgct

SEQ ID NO: 22 - DNA, 490 bp, Homo sapiens
  misc_feature (434)..(434): n is a, c, g, or t
cccaacatttcctgttatgccgtcatacctccggtgttcagtttgttactcaaaagttcccagttcttaggctgaaaaacattcaagtaaaaatgtagcatagattacactgaaatattttcagtatcaatgatttaatatccacacaaactattaggaatggtatctggaacttgttacaaggtcagataaactgttacagaattttagaaataattatctctgtatgtgactgtgtattacatataaaaatgaatttttcaggaaaagtgcactttaaaaatactttctcttgctagttttttaaattgatgaaatagcctgtgctgctttggaatgcttattctttctttgatgaaaatacggtcattgattatgataattttaaaaatatctatttctgcatcgctgcaggatttcctacacttttctgntcctcatccctcttttatgtgtgcttggctcagttgcctggtacacagcccctgcc

SEQ ID NO: 23 - DNA, 445 bp, Homo sapiens
  misc_feature (444)..(444): n is a, c, g, or t
gtagcaaataccgaaacaatagttattgaccacagtataccaaatggaagagaccagcacctgggcgtggacccaacagagcatttatttgagaatggcagtgagtttccctcagagctggaggacggggacgacccagcagcctacgtcaccaacctgtcatattaccacctggtccccttcgagacagacatttgggactgaacctctctatcaggcctcccccgcctcagctgttcactgccagctctgatgctgtccactgtgctggcccccatggtcacagctgctgacccagctctggaactcccagacgtcccaggccccactgacgccaggaggcctgggtgtcctgcaagtggagcagcaggggctcctagtgggccaggctgaccgcagagtctccagcaagctgaggtcacagccggaggccacagggcctgng

SEQ ID NO: 24 - DNA, 357 bp, Homo sapiens
  misc_feature (356)..(356): n is a, c, g, or t
ggaaaatttaaaaaaaaactaaaaaaagcataatcgcaaaaatgacacaaatgtcaaaaaaatgtagacataaaagactggaaagataataacggccgagctgtcatagaaatcacagctaaaatgcgcaagttagcccatgttccagttcagacttgctccagataatccccggattccttgtttagaggatggttctcaacctttaaaagattcaaaaccctcattggtgggtttttgtttattcttggaatctgcctcatcctcccttgacttttacccctgtaagtaggagtatttgtcaactatagaggcagtggtaacccgacccactgccccgcagttgatggcattanc

SEQ ID NO: 25 - DNA, 200 bp, Homo sapiens
aatgttgaacttcatgatgcatgaccagcgcaccggcccggcatggaacgtgctgatgtggaccatgctgtttctaggccagggaatccaggtcagcctgtactgccaggagtggtacgcacggcggcactgccccttaccccaggcaactttctgggggctggtgacacctcgatcttggtcctgccatacctagaggt

SEQ ID NO: 26 - DNA, 209 bp, Homo sapiens
  misc_feature (144)..(144): n is a, c, g, or t
tgttgtgtcacaaagaggccccagtctctttcttcttacatccatgcctcttactagatgatacatttacagattgggcagtttgatctcaaaacctgggtgagaagactattcctaggactctagcaacttcaaaactgaggntggagtttcagaatctttttctgcatcaattcagtctattggccgttcaacaaagagaagtcagc

SEQ ID NO: 27 - DNA, 200 bp, Homo sapiens
ggtctgaccagtttcttcttcacccggcacgtatgggaaccgcgggcgctgaaggcgccggagtcgcgcgacggatttttgacggcggtatcgcgatcgatagcaaggacggcagggcagggccaggccgcgcaggaaaggtcagcacaaccatgagttcagataccatgcctgccatcgcgtttcccggtggtatcggc

SEQ ID NO: 28 - DNA, 279 bp, Homo sapiens
  misc_feature (17)..(17): n is a, c, g, or t
gggcagccgggatgacngctctccccaggaatcctgctgcctgctgagaaacatggtcagcaagtcccgctggaagctcctggccatgttggctctggtcctggtcgtcatggtgtggtattccatctcccgggaagacagtttttattttcccatcccagagaagaaggagccgtgcctccagggtgaggcagaggagcaaggcctctaagctctttggcaactactcccggggatcagcccatcttcctgcggcttgaggattatttctgggtcaga

SEQ ID NO: 29 - DNA, 531 bp, Homo sapiens
gagcaattctcattcctctcagccaaaggcctgcactgggctttatttgtctttttttattttttaagcactgcctgccagagatgggcctggggcctgatgaggaccttagcgctgctcgttctccttttctgttcatgcacacattcctccatggggtggggaaggcaggcatggggtgtggccctcggagaagttaggagtcccccagctcaagatacagtggcaaagacctagtggtcccctacccccacttctctcagttcctggcatgaggagagaagaccctgctctggtggagctgacaacctttgaggctgggaggagagcagcctctgggcatcgttcccagtgtccctcacactaaaacggcgtagatggcaaccccccacccccaccccgctgctcaactcttgtgtttgttgttctgtttgccccatttatctgttgctgtttttgtgttgtcttcccctgctccgcatttgtaaatggccccggggggaggtttttgctggatctgctcctctcgtt

SEQ ID NO: 30 - DNA, 200 bp, Homo sapiens
ggtttaagcctacaatcattgattttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgcta

SEQ ID NO: 31 - DNA, 482 bp, Homo sapiens
  misc_feature (297)..(297): n is a, c, g, or t
ggtcgcgctcagtggctatgaccaggtgctccacgaagacgaaaccacgaaccgcatgcacgagtctctcatgctcttcgactccatctgtaacaacaagttcttcatcgatacctccatcattctcttcctcaacaagaaagatctctttggcgagaagatcaagaagtcacctttgaccatctgctttcctgaatacacaggccccaatacctatgaagacgcagccgcctacatccaagcacaatttgaaagcaaaaaccgctcacccaacaaagaaatatattgtcacatganttgtgccacagacacgaataacatccaggtggtgttcgacgccgtcaccgacatcttctttgccaacaacctccggggctgcggcttgtactgacctcttgtcctggatagcaacctatttgactgcttcacggactctttgctgttgacgttgatctcctggtagcatgaccttttggcctttg

SEQ ID NO: 32 - DNA, 200 bp, Homo sapiens
tggtgagattcgttggtggctgcgcgattgtggcggagttgcaagcaaacgggtttcatcaccttaaatggttttgaaccaaagaagctgtattcccttaaaaagacggacaacccatcgtgtgaactatagagtttgtgaacaaatttatattgggttcatagtggcgtcatgcacgcagactcctgcgagttccccta

SEQ ID NO: 33 - DNA, 400 bp, Homo sapiens
cacgttagtccccccatccatctagaagtccattttgaaagatttttgtaaattcttttaacactgatgtttcagcctcgtctttgtttcagttaagctcaatggcgaacatgggaaccacctttcgccttccctgggggagaaaccctcttggctgatggcttttccccggaattatcaaacagccaccgggtgactttctggcttccagatccatctgcctgagacccccgaactcctctcctcccaagcagaggcgagtgagtggcattagctcccggacccattcccggtcctagctgggcatggggctgacggagatgaccaagccttggtctgctctctagcagcttccacagacttggctcgtggccttccttatatccactgggaacaaaca

SEQ ID NO: 34 - DNA, 485 bp, Homo sapiens
  misc_feature (453)..(453): n is a, c, g, or t
ggggtgctttctcacatgtcttagtaggctgtgcaggctggaagtgctcagaagtcacacccccagggagcagcctcagccaacagcaccttggctgtaaatgccccagctccctcgccctcaggtaagcattgctgaggcacacgttccatactcttttccacagttcctccgtgggactgagcaccacccagccacccacaggagcagctaacctgataaccaccagcctcaccctccctgccttacttccccgctcccctttaccacatgctgacctcccagatgcatttcttgctttccggtctctgtctcaggattggctcctggatgaacacaaactaacactatgttcacaaatatatttgggaaatgctggatgaataattatacacatcagacagattactagaaattctcaccaaagggatgcacatgttacctctgcatggngagatctcaggtgctttttaccccacatagct

SEQ ID NO: 35 - DNA, 443 bp, Homo sapiens
gtctacttgaaggacacaatgttttccaaactagcacatttgcagaggtttgctgtacttagtcgcggagttcattcttcagtggcttctgctacatctgttgcaactaaaaaaacagtccaaggccctccaacctctgatgacatttttgaaagggaatataagtatggtgcacacaactaccatcctttacctgtagccctggagagaggaaaaggtatttacttatgggatgtagaaggcagaaaatattttgacttcctgagttcttacagtgctgtcaaccaagggcattgtcaccccaagattgtgaatgctctgaagagtcaagtggacaaattgaccttaacatctagagctttctataataacgtacttggtgaatatgaggagtatattactaaacttttcaactacccaaagttcttcctatgaatacagga

SEQ ID NO: 36 - DNA, 466 bp, Homo sapiens
  misc_feature (450)..(450): n is a, c, g, or t
cccgagcccaaaagcttcaattacccattgctctcgtccccaggtgatcagtttgaaattcagctaacccagcagctgcagtccgttatccccaacaacaatgtgagaaggctcattgctcatgttatccggaccttgaagatggactgctctggggcccatgtgcaagtgacctgtgccaagctcgtctccaggacaggccacctgatgaagcttctcagtgggcagcaggaagtaaaggcatccaagatagaatgggatacggaccaatggaagactgagaactacattaatgagagcacagaagcccagagtgaacagaaagagaagtcgcttgagttcacaaaagaacttccaggatatggctataccaaaaaactcatcttggcgttaattgtgactggaatactaacgattttgattatacttctctgcctcattgagatctgntgtcaccgaaggtcat

SEQ ID NO: 37 - DNA, 482 bp, Homo sapiens
  misc_feature (448)..(448): n is a, c, g, or t
tcagaattagcctgggcgacaagagcgcaactccgtctcaaaaaataataataataaaataaaataaatttgtattttattacaatagcatttgcagataggaaactcgtgcttattattccttttatgaatttattgacttatggaatgcctaatatgtagcaggcactgaggtaggcactgaagacacacaggtaaaaccaatacggcgcctgacctcatcgagcacagatacatgcaagatgtgctatttattgtgtctacagacaatgctcacaggtggatgggagcaccctgtctctgactcctcagaattagcagttgcttccttctttagccagcaaacttctgctgtatacattataaacaaacacacaggtcaggaataatacttggttaccagtaatgtgataagtgtgaacacccaaatgcccttgacaatgaaagnactgatacgtgttgattggccctctaggtctgga

SEQ ID NO: 38 - DNA, 122 bp, Homo sapiens
  misc_feature (114)..(114): n is a, c, g, or t
gctaaatatataaaaagacttataaaaactagaattttatccaaacattttgtgcaactaatgctaaaatattttaagttaaattttctttttcttttttttttttttttaaangaaaaaaa

SEQ ID NO: 39 - DNA, 541 bp, Homo sapiens
  misc_feature (520)..(520): n is a, c, g, or t
gggggagtaaggtcagttcaagcagaaggagcattgctaccaaaggattggagaatgaatagcaatagggtatgtttcagtactggaagttgcaaatgattgtgttaaattgagggacagcaaccagtttcctcctgtgccatgctcttgagtgtggtttctgctttagtcactacaaatattagtaaatgtattcaacaactaatgctttctcagtgatgtagtaatagtaatgtttttctttttgacattttcatgtttttctttacttttttttctataggtatttggtgttcatttttttaaattttagagcttattatttatactctacctttcaaatcaccaaatatgatttcgttattataagtataaaatacatttagttcagtgtgttttgaagctgaagtttaacagtagtgctttaaagttgagtcttatagaaaataggatatcatgaacggctgatatatatttttaaaagttgaatttgtattggtaggtgttgtctacatctacnggcctaacaaaacagtgtata

SEQ ID NO: 40 - DNA, 470 bp, Homo sapiens; identical to SEQ ID NO: 6
  misc_feature (466)..(466): n is a, c, g, or t

SEQ ID NO: 41 - DNA, 375 bp, Homo sapiens
  misc_feature (7)..(7): n is a, c, g, or t
ctagcantgtgatgcaagtggtgtancttaatttcaaactggaagttaatttgtagaagaaattaaaacctgttttgccttatctgaaacatcagaatggggtgaaaggggttgagaactgtgtttttgctttaaaactgatttttgttattattgttctagatggttgaaccactaagagagaaaatcagagatttagaaaaaaggtatgtgtactttttgtcttaaagtcaatagttaaaataagtttgtctcaattgagtaattaattctcagtttgtttgctccaaactctgccaacctaaataaatgtctatatacgcatttcccccactttgagtgtaatacccagatagaacatggggagaagaatca

SEQ ID NO: 42 - DNA, 318 bp, Homo sapiens
  misc_feature (172)..(172): n is a, c, g, or t
gggtttccagaccgaatactaccaagaaaattcgctttaacaacaaagattcctgatacagaaaggctgccacaaatgttgcatagtcagaaacccttacacgggacataaatacctctgtggagctttacagtctggaattgttttacttcagtggtatgagccaatgcanaaattcatgttgataaagcactttgattttcctttgccaagtcctttgaatgtttttgaaatgctggtgatacctgaacaggaataccctatggtctgtgtagctattagcaaaggcactgaatcgaatcaggtagttcagnttga

SEQ ID NO: 43 - DNA, 533 bp, Homo sapiens
  misc_feature (508)..(508): n is a, c, g, or t
gaaaaaaagaaaaaatccacaaaagccactttattttaaaatatcatgtgacagatactttccagagctacaacatgccatctatagttgccagccctggtcagttttgattcttaaccccatggactcctttccctttcttctctgaaaaaaactaatttaaatttgcttttcttttttttaactgagttgaattgagattgatgtgttttcactggatttttatctctctcaacttcctgcacttaacaatatgaaatagaaacttttgtctttactgagatgaggatatgtttgagatgcacagttggataatgtgggaaaatgacatctaagctttacctggtcaccatgtgatgtgatcagatgcttgaaatttaacacttttcacttggttcttatactgaatgccgactctgctctgtgttagagatatgaaatggtgtttgatactgtttgagacattatggagagatttaattatttgtaataaaagatttgctgcagnctgaaaaaaaaaaaaaaaaaaaaaa

SEQ ID NO: 44 - DNA, 445 bp, Homo sapiens
  misc_feature (416)..(416): n is a, c, g, or t
gaggatattcaaggagttcttgtttggtctgactctttaacttttgttgtccagttgtatattatataagacccagtagcatgacaaaattgccagaattttgcttttccagattacatagtctttcctgtgggctctgcaatctccactctccctccaaaacaagtacatttttgtgaaattttaaatagaaaaatctctatatagatctaatgtcggtttcttaacatttacatttcagtgtcttgggatttgtgattacatcaacttgttttggaaaagaaataaattctcttaactttcttttaaaaaaatctattttaagttatagagatgacataacaatattatagacagtttaccaaaaatgtgataatcctgccacccagtcagaatagacattgccatttttctgnttcctttcagactttaggtgacaggacag

SEQ ID NO: 45 - DNA, 425 bp, Homo sapiens
  misc_feature (423)..(423): n is a, c, g, or t
ggagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggngg

SEQ ID NO: 46 - DNA, 475 bp, Homo sapiens
  misc_feature (20)..(20): n is a, c, g, or t
gggtaggtcagctctgaaangcacccggtgctgcggggcctgccttcctagtggcttcggcttgggcgccatcccaagccgggttggccagaactctgcctgtgcagccccgcccccaggcaagcaggaaacaatgcacctgagcaacctcctcccatctgcagaggaggaagctaaggtgtaggggggtcaaggcactggcctaaggtcccacagccaaggggtgggctgggaggtgcaatcaggtctgcctggcacagagtctggtctcctggaccacgatgccaaactggttaccgccgtcaccacaatagaatactaataatagttgtcgtaattactacttactggtggagggctgttctccaggcaggcaggtctgggttgctgcgtcactgggggccaggaggctggctagtgcccacaatgaggcaggggtcctggtcccaaacagcactaggaaaattgctggnag

SEQ ID NO: 47 - DNA, 481 bp, Homo sapiens
  misc_feature (295)..(295): n is a, c, g, or t
ggcccaatgcccaggcccagatgtaccggctgaccctgcgcaccagcaaggagcccgtctcccgtcacctgtgtgagctgctggcacagcagttctgagccctggactctgccccgggggatgtggccggcactgggcagccccttggactgaggcagttttggtggatgggggacctccactggtgacagagaagacaccagggtttgggggatgcctgggactttcctccggccttttgtatttttatttttgttcatctgctgctgtttacattctggggggttagggggantccccctccctccctttcccccccaagcacagaggggagaggggccagggnaagtggatgtctcctcccctcccaccccaccctgttgtagcccctcctaccccctccccatccaggggctgtgtattattgtgagcgaataaacagagagacgctaaaaaaaaaaaaaaaaaaanggggggggga

SEQ ID NO: 48 - DNA, 465 bp, Homo sapiens
  misc_feature (437)..(437): n is a, c, g, or t
gcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgnaacacaagtagatgcctgaacttgaatt

SEQ ID NO: 49 - DNA, 527 bp, Homo sapiens
  misc_feature (510)..(510): n is a, c, g, or t
gcccattggtagacgtgtgtttctagagcctgctgtgatctcccagggctactcactcaagttcaaggaccaacaagggcagtggaggtgctgcattgcctgcggtcaaggccagcaaggtggagtggatgcctcagaacggtcgaggtaatgtgaactagctggaattttttattcttgtgaatatgtacataggcagcactagcgacattgcagtctgcttctgcaccttatcttaaagcacttacagataggccttcttgtgatcttgctctatctcacagcacactcagcacccccttctctgcccattccccagcctctcttcctatcccatcccatcccatcccatcccatcccatcccatcccgctcttttcctacttttccttccctcatagcttccattccacatccggaggagaagaagtaatgaatttctctacagatgtcccattttcagactgctttaaaaaaatccttctaatctgctatgcttgaaggcccgcgntacaaaggaaaagtatc

SEQ ID NO: 50 - DNA, 447 bp, Homo sapiens
  misc_feature (425)..(425): n is a, c, g, or t
ggtaagttgtgttctagtctttgactaaaatttatcatctcttataatgggacttaatctttctctaaaagcatataagagcttgtcaatagagcaatcaatcaaaaagattttgtgattcataacattgaagttagtctggttaagagttttggtttagacttcatttatattttccttactaatatctaatatttaatgaataatgatcaattttttataaagttattaatatgatcagggaaacctttgggacttctgacaggcatctggtgaagagacaattcaagccttagtgactatttagaatagccagtgatcactagctaattctcatatccatgcctttttgtcctgtttacagtcttaaaagaggtaaaacagcaaatatttttttaggggaactataaccttaggattcctgnaaagaattcaaaaaaaaataaa

SEQ ID NO: 51 - DNA, 425 bp, Homo sapiens
  misc_feature (389)..(389): n is a, c, g, or t
gggggttgatacctgagcctgcaatagagccctgaaatcaagagcatagcttgagtgtgtgaatatgatgtgtgcacatgcttaatgagcgtgcaagtgtgcacacgtttgtggagaggagggtgttctggcctgagaaggtaaagaagaggcatgtccagtatgctttgcagggtgtgtttgctcttttccatgcccatgcaacccagattggggtggagcaggaaggagctcttttctgttcccaagcctcagaactcttgagctgtggcttacttgctgtcttcaccaggttcaagctccgtgggccacactgctgctgtgccaagaaggtgtacagcctccccaggatggggcctcatacaacccttcatctgcactcaacattnaatcgtgtccttgctgnctttttattttcctttttg

SEQ ID NO: 52 - DNA, 325 bp, Homo sapiens
  misc_feature (317)..(317): n is a, c, g, or t
ggcgtctttgaaatgctgattttttttcactttaaaaaaatgactttttttttaccactgatagtatttcatatggtccctcagtaatatttcttagagatggttaaaaataggttctgagtcatcttaagcatatttctaggaatttgttttattgctgtcatgaatataatggttttgctccagttcatttcatagtttaacatatcaaatattaggtatcttaaaaatcacaaatgctatttcaccctgcaggttagcagtattaaaatagaatcatcaaatcccccatcagtgggggcgggggggggggttgnaaaaacag

SEQ ID NO: 53 - DNA, 533 bp, Homo sapiens
  misc_feature (448)..(448): n is a, c, g, or t
ggatcttttagaaatgtttgcacctttggtggtggcacaggaaaagagaagaattcagctggagtgaattctagaagtagatatcagaaacggggcatgaagaacaggggaactgggtggcatcagactcctaaagaagtgagttaattttccttcccttccattcagattcatgccacagctccatatcttgagtatgtgtaagaggtgagttccttcttcagccaggggcggtggctcatgcctttaatcccaatgctttgggaggccaaggtgggaggatcacttgtgccttggggttcaaggttgcagtgaaccatgattgcaccactgcactccagcctgagtgacagagcaagaccctgtctctaaaaatatatataaaaagtaaaactaaagaacttcttgcctaaacctgaattaccgcaatttgctgagtgactttganaaaatcagactgnttagttcagtcgggatgaaaagcttgcgatgctnccacagaatgggcaatagtgacggctgcaaggtacttt

SEQ ID NO: 54 - DNA, 415 bp, Homo sapiens
gtgaaaattacaaagcgttaaatacaaaagacaattctatacaactcaacagaataagaaggaagtatatacatcactttttaataggtgcagagagacttgcaccaaacttcaggattaagcaatggaaaatgtgacaaggcacatgtccgtcgcagtgaaatttcagaattcaagtgattccaggcaagaggccaaacttaacttggcttctttcaacttaaacagccccctttggcataagtccacactgaattttaaagtgaacagagggcctttttcccccaaacataaatttcctttagctgtatgtcaaagcggactgataaaccagctcctacactgttaacccttccatggcaacagtagttattgctgtgtctccaatccatgcagagcatttttctttaaaaaa

SEQ ID NO: 55 - DNA, 236 bp, Homo sapiens
  misc_feature (157)..(157): n is a, c, g, or t
gacgcagtactcctgggagcgccaggcggccttccgcttcaaaaggaagctgcccaagtacctgctctttaccagcccccaggagaacccctggggccacaagcgcagctaccggctgcagatccactccatggccgaccaggtgctgcccccaggntggcaggaggagcaggccatcacctgggcaaggtaccccctggcagtgaccaagtaccgggagtcggagttgtgcagna

SEQ ID NO: 56 - DNA, 509 bp, Homo sapiens
  misc_feature (51)..(51): n is a, c, g, or t
gcgcaagttggggctgcgccctgggatacatcattcactctttggggacgngaagaagctcatcactgatgagtttgtgaagcagaagtacctggactatgccagagtccccaatagcaatccccctgaatatgagttcttctggggcctgcgctcttactatgagaccagcaagatgaaagtcctcaagtttgcctgcaaggtacaaaagaaggatcccaaggaatgggcagctcagtaccgagaggcgatggaagcggatttgaaggctgcagctgaggctgcagctgaagccaaggctagggccgagattagagctcgaatgggcattgggctcggctcggagaatgctgccgggccctgcaactgggacgaagctgatatcggaccctgggccaaagcccgnatccaggcgggagcanaagctaaagccaaagcccaagagagtggcagtgccagcactggtgccagtaccagtaccaatacagtggcngtgccagtgccagcac

SEQ ID NO: 57 - DNA, 514 bp, Homo sapiens
  misc_feature (491)..(491): n is a, c, g, or t
ggctaaggggcttggctcttgtacgggatggatgatgtgagcgcacagttcgtagggtcaggcgtcttgcggtaggaaaggggcagggttcgatacctctggggtgtggcgtgttctctcttgcccacctccccaagctccttttaaccaggagcttccctcccagagtgagctacgtcttgggaggattgaagcagggaggtggcagtggctagagttgctcagatcggctcagatgaaggcccaggggtcaaagcatttgctcaccacaactgagtctggaaggcactgtgcagccaaaccctcttgtccaatcacatgcaggccccaggcccctcagaaacgccctggtggagggaggagcccaacagatagggtcagggagtcagtggggaaagcagaggggagagcttagagtgaaacttgaaactgctatggacatggaggtcagatgggaacttggaactgggcatgaatcctgaatggggggnaacttgaagccatcacagccaga

SEQ ID NO: 58 - DNA, 482 bp, Homo sapiens
  misc_feature (461)..(461): n is a, c, g, or t
gggcaaatggtatgaattacaagacctccaggtgactgacatccttccccagatgatcacactgtcagaggcttacattcagatttggaagaggcgagataatgatgaaaccaaccagcagggggcttgaaggaggcgtctagggctttgctcccaagggctgtggctgatgatggtaaataagaacacagaagctgtagctgaacacaggctggctggtgggcttcctaggccagcccagcttgtatgggttctggctacaccagagcaccaagagcccacttgcctgggatggccccacactgtcactcagctgttctttgatcatttttttctagattgatgctcctttctcccatgcattgagctcccatctagcttcagcagggcagaacccttctccagatgtgtgtaacttatgtctgagtatctgggagtagttgaagaacagataattcctnccaacatcaagccttgggatt

SEQ ID NO: 59 - DNA, 510 bp, Homo sapiens
  misc_feature (485)..(485): n is a, c, g, or t
ggaattagcacagttaagttgacactagaaactgcccatttctgtattacactatcaaataggaaacattggaaagatggggaaaaaaatcttattttaaaatggcttagaaagttttcagattactttgaaaattctaaacttctttctgtttccaaaacttgaaaatatgtagatggactcatgcattaagactgttttcaaagctttcctcacatttttaaagtgtgattttccttttaatatacatatttattttctttaaagcagctatatcccaacccatgactttggagatatacctataaaaccaatataacagcagggttattgaagcagctttctcaaatgttgcttcagatgtgcaagttgcaaattttattgtatttgtagaatacaatttttgttttaaactgtatttcaatctatttctccaagatgcttttcatatagagtgaaatatcccaggatacctgcttctgtgncagtcgcatttgacgcataactgca

SEQ ID NO: 60 - DNA, 558 bp, Homo sapiens
  misc_feature (494)..(494): n is a, c, g, or t
gttttgtttttgtgtggttcttgagctctctgacatcaagatgcatgtaaagtcgattgtatgttttggaaggcaaagtcttggcttttgagactgaagttaagtgggcacaggtggcccctgctgctgtgcccagtctgagtaccttggctagactctaggtcaggctccaggagcatgagaattgatccccagaagaaccattttaactccatctgatactccattgcctatgaaatgtaaaatgtgaactccctgtgctgcttgtagacagttcccataactgtccacggccctggagcacgcacccaggggcagagcctgcccttactcacgctctgctctggtgtcttgggagttgtgcagggactctggcccaggcaggggaaggaagaccaggcggtaggggactggtcttgctgttagagtatagaggtttgtaatgcagttttcttcataatgtgtcagtgatggtgtgaccaggcagcatctancaaaagccagcatggagtaggtgaccgatacttgtcatgactaatatacaataaagagcctngg

SEQ ID NO: 61 - DNA, 399 bp, Homo sapiens
  misc_feature (392)..(392): n is a, c, g, or t
gcctatgaagactttgttgagggtctgcgtgtctttgacaaggagagcaatggcacggtcatgggtgctgagcttcggcacgtccttgccaccctgggagagaagatgactgaggctgaagtggagcagctgttagctgggcaagaggatgccaatggctgcatcaattatgaagcctttgtcaagcacatcatgtcagggtgaagcagagtcttccaggtgcctggcccttggctttagccataccagggtgagttaaagagaggccccggctgggtgagctgagatggagtcctcgacttatcaccacaccactgccccaaggaccttacaggccctccctgttaataaacagctctaacacggccaggctgggctctgggattctgaanaaaaaaa

SEQ ID NO: 62 - DNA, 393 bp, Homo sapiens
  misc_feature (374)..(374): n is a, c, g, or t
atcagattgtgcaagcttgaacgcttgtttccctcccataataacaatgccacatgttgcttgtgtgataaataatctttgtttactgtggacacttcggcaatttggttcatcttggtacagaaaatacaaagacaggactaatattttattcattttttatacttagtaactaggacagggatctgtatttagactatattcaagcctcgctctgtcacccatgcttgagtgcaacctctgcccactgtgttcaagcgattctcctgcctcagcctcctgagtagctgggagtacaggcgtgcatcaccacacccggctaatttttgtatttttagtagagacaaggttttgccatgttgaccaggctggtntcaaactcctgacctcggg

SEQ ID NO: 63 - DNA, 510 bp, Homo sapiens
  misc_feature (237)..(237): n is a, c, g, or t
gtgtatttttagtacagacggagtttcaccgtgttagccaggatggtctcgatctcctgacctcgtgatgcacctgccttgacctcccaaagtgctgggattacaggcgtgagccactgcgcccggccaataatttttttagtttaagttcatttttgtccccgctgatgaaagttagagctgcttcctccaggcagccctgggagtaggggcacagacttgtgcctcgcttcccancagctgcccagtgcctctgtccacacccacctccctggtgtacttgccccctacagcagcagcccagatcctcccctggattaaaattgcaactggtgccctaacccaaaacttgagaaaaattctctatcacatccactcttctggnatttgtagaatctctggccctgggtgtgtcttttcctgtagctgttaaaaatttcttaagcagatttcttagcagatggacatgtccagattcttgcctaaatatccagntgataactggaaatg

SEQ ID NO: 64 - DNA, 500 bp, Homo sapiens
  misc_feature (498)..(498): n is a, c, g, or t
gaggatgaggagggggaggaggaggaagagtatctggacaagatcaaccccatctatgatgctctgtcctatagcagcccctctgattcctaccagggcaaaggctttgtcatgtcccgggccatgtatgtgtgagctgccatgcgcctggcgtctcacatctcacctgttgatcccttagctttcttgccaaggatctagtgccccctgacctctggccaggccactgtcagttaacacatatgcattccatttgtgatgtctaccttggtggctccactatgacccctaacccatgagcccagagaaattcaccgtgataatggaatcctggcaaccttatctcatgaggcaggaggtggggaaggtgcttctgcacaacctctgatcccaaggactcctctcccagactgtgaccttagaccatacctctcacccccaatgcctcgactccccaaaatcacaaagaagaccctaaactataatttgtcttcaggnag

SEQ ID NO: 65 - DNA, 487 bp, Homo sapiens
  misc_feature (458)..(458): n is a, c, g, or t
ctaggaatagccccctttcacttctgagtcccagaggttgcccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggctggccccgctaacnggtttttgcccaatgggccatttcgaaga

SEQ ID NO: 66 - DNA, 431 bp, Homo sapiens; identical to SEQ ID NO: 12
  misc_feature (16)..(16): n is a, c, g, or t

SEQ ID NO: 67 - DNA, 477 bp, Homo sapiens
  misc_feature (221)..(221): n is a, c, g, or t
cctttccccaggagaagccaagaacttgtgtgtcccccaccctatcccctctaacaccattcctccacctgatgatgcaactaacacttgcctccccactgcagcctgcggtcctgcccacctcccgtgatgtgtgtgtgtgtgtgtgtgtgtgactgtgtgtgtttgctaactgtggtctttgtggctacttgtttgtggatggtattgtgtttgttagngaactgtggactcgctttcccaggcaggggctgagccacatggccatctgctcctccctgcccccgtggccctccatcaccttctgctcctaggaggctgcttgttgcccgagaccagccccctcccctgatttagggatgcgtagggtaagagcacgggcagtggtcttcagtcgtcttgggacctgggaangtttgcagcactttgtcatcattcttcatggactcctttcactctttaacaaaancttgcttc

SEQ ID NO: 68 - DNA, 178 bp, Homo sapiens
gaaaaactctgaagaatcccctccaaccattctcttgtgaacagaagaaatcaagtccctcttccctcctccctcttcattcctgtccccatggggagaacaaatgctactgttcttgtccccttttttagatatgtcaaaaaaattaagttttctagtcctgttctttttttttttt

SEQ ID NO: 69 - DNA, 566 bp, Homo sapiens
tttatttgtccatattgttattttctcttacatgtgctacttcaaaacaaataccaatctgctgatgacagtcccttgctgaaaatgcttcagtggctctccatggcccttaaagtcaagtacaaagtctacaagcggaaaagtgatctgcaaagcccttcaggatctgaccccaggtcacttgtctagtcaagggatggctattttttgttgttttgcttttcagttcctcaaatattcaaagctttcttttgcctctgagcatttgttcctactgttccttctgcttctgtatctcttcatcttggtccgattagcccttactcatatccttccagtttcagtctaaatgtcactcgttccagaaaccactactgacccccccaagtctatgtcatgcaccctttcttcttggcatctgacacttcttggcatctgacacttggcgaaacttccctgttttgcagcttttacccactttgccatagctgcctgtttacttgtgtgtattctttcaaatactctgatttttccagagggcaggcagtgactcttcttcaatattg

SEQ ID NO: 70 - DNA, 569 bp, Homo sapiens
  misc_feature (562)..(562): n is a, c, g, or t
gttataatacattaacagtaaataatatctatgtaaatttctttcaccatgctgtctcagagtcagcagtgaacaaaggcaaagattggggccccacttagtttgaaaaagggaaaggaagtgatctaggatgtgtactagaagcatttcaaatgttgggtctttaaaatgttttgttcagtcttagcaaatcttcgatcttttatttcaaaattccagtagtccactgtggagcccatgcatttcatctggaatttaatgttactttgaagccaaaccatttaaacaattatgtgtgttttattagctagtgttgctgaaaatgcacttagttcaattttaattgtaaatgggaattttaaacttttcctagttagcatggttatatgttagttaaaactgaaagctaaattgaagatattacattttaatttatggtgacagataagggtttgtatgcattatgtgtaaatcagcttcatgctgtttaaaatttattctctgaaatttatagaccatgtagaatgatttgtgatgtgatgctttatttattgcaagaaantagtatg

SEQ ID NO: 71 - DNA, 506 bp, Homo sapiens
  misc_feature (8)..(8): n is a, c, g, or t
gaaggacngtcctangaggagaaaagtaagacggcggcgagatcaggaagatgccctgaaccaaagataccctgagaacaaggaaaaaagaagcaaagaggacaaagaaattcacaacaagtacacagaaagggaggtgtcaacaaaagaagataagcccatacagtgcacacctcagaaagccaagccaatgcgggcagctgctgacctggggagggagaagatcctcaggccaccagtagaaaaatggaagagacaggatgacaaagacttaagagaaaaacgttgttttatttgtggaagagaagggcacattaaaaaggaatgcccacagtttaaaggctcttcaggtagcctttccagtaaatatatgactcagggaaaagcctcagcgaagaggacccagcaggaatcatgagggaaggaaaatgcagcactctaaatggccactcaggcgttnctattcactcggaaattaggttcatttcacaggacacagcagtgta

SEQ ID NO: 72 - DNA, 303 bp, Homo sapiens
ggaggaacctcagagccgccggacacggatgtcgttgggcaccaaggggctgaaagtcaacctctttcctggcctgagcccctcagccctgaaggccaagctgcgcccccggaatcgctcagctgaggagggagagctggctgagagcaagtcgagccagaaggagtccgcggtccagcgttcgaaatcctgcaaggtcccaggactgggaaagcccctcacgttacctcccaagccagagaaatcctcagggtcagaaggatcgtcgcccaactggcttcaagccctgaaactgaagaagaa

SEQ ID NO: 73 - DNA, 498 bp, Homo sapiens
gcgcagccacaggctcatcctctcctccaccagaagtgagggctcactggcttccaagcaccccatagtcatttattggctcaaaacctctcttcttaggcatccaaacctcagaggagtgagagaaaatggtcaactgattagtctgtactccagaactaaatcacctgcctcaaccatcagagtcagtggaaaaaacatgtccaggcctccccctcagatcttagcccccatcactgaacctgaaatcctgtgtttcccaaagtgtccatgtcactgtcatgagaggatcaaggagaggaccttttctttccccactcacaccctacaccagcacaggcccagcgagacagacacacactcaggagctctcacataacaaatgaaggaatgggaatgaaatgaatgatccaaaacctctttaaagactgaatttgcatgcagaatcctaggagtttctagccacacctgtccttgtcttcagaggctgacaccatg

SEQ ID NO: 74 - DNA, 451 bp, Homo sapiens
  misc_feature (412)..(412): n is a, c, g, or t
gagtggttaagaatgtatatcagggtttaacaataggtatttcttccagtgatgttgaaattaagcttaaaaaagaaagattttatgtgcttgaagattcaggcttgcattaaaagactgttttcagacctttgtctgaaggattttaaatgctgtatgtcattaaagtgcactgtgtcctgaagttttcattatttttcatttcaaagaattcactggtatggaacaggtgatgtggcataaggtgagtgcacggtatgttcagatcacagtgccttatgtccgaatacagcaatatgtcaccgccgcagccggggcgcacgcgtgtgaaacaacaccgagcttgaatgtggaagtctttgaaccttttaccaaatcagtttgttttctttagatttgtcaaaagttgtanttgaatataaataattactttaaattgtaatgacctttt

SEQ ID NO: 75 - DNA, 594 bp, Homo sapiens
  misc_feature (25)..(25): n is a, c, g, or t
gacagcctccacccctgacccccancctctnntcctacctctctgtttggtttagaaattgctcaataagtcatttggggtttggcatcctgcccagccacttcccaaacatgaagacctctctgttgctttatgttgtacatgccccatagccatcccaacgtcctccccagtcctctcctggcaccagcaccttagaagttgttggcagaaggcacttaaactgtgggagaagtgtgcacacctttgagtcccttccctcaaggttaaagctcctgtcagactctcagaagggtctgtgggtgttgtatattaggcaaacaggggaaagcttagaggtccttctatatgtgttaataagctgtttctaagtgtttaaatttgaaaagcatcatgttctcatgatttatgggaatgaagcaagtactgaaatcaaattaaatactccctgggtcctgggtcagtttgaccctagccctggggtgaggcaagccncctcctatgaggatgagcaaaaatactactctcttcgccctgagttgctttctggatctggggcttcaggattgctgcttcagtcagcctttatancac

SEQ ID NO: 76 - DNA, 324 bp, Homo sapiens; identical to SEQ ID NO: 14

SEQ ID NO: 77 - DNA, 614 bp, Homo sapiens
  misc_feature (471)..(471): n is a, c, g, or t
gctcgcgcgggtgtcgctgggctgtcggcctggaccttgcagccgcaatggattcaagttcgaaatatggcaactttgaaagatatcaccaggagactaaagtccatcaaaaacatccagaaaattaccaagtctatgaaaatggtagcggcagcaaaatatgcccgagctgagagagagctgaaaccagctcgaatatatggattgggatctttagctctgtatgaaaaagctgatatcaaggggcctgaagacaagaagaaacacctccttattggtgtgtcctcagatcgaggactgtgtggtgctattcattcctccattgctaaacagatgaaaagcgaggttgctacactaacagcagctgggaaagaagttatgcttgttggaattggtgacaaaatcagaggcatactttataggactcattctgaccagtttctggtggcattcaaagaagtgggaagaaancccccacttttggagatgcggcagtcattgcccttgaatactaattctggatatgaattgatgaagctcatcatctttataaatcaggctgnatctctatagaagagaaagccatctttccttatacgtggagggtgaagaga

SEQ ID NO: 78 - DNA, 307 bp, Homo sapiens
  misc_feature (208)..(208): n is a, c, g, or t
ggtcgccagtttctccagcactttcttcgtccagggacgtcaacctcccttgcatgctgtcctcagggactgacagcggggcctgctcagaagttctcgtcgggactctctacaggcctttgtgattaagtgctaggggtgaggagagacgtggcttatttatgtgtttttcagtttgttatgattttttttttttaattttacttcnccctgccctaaaattgagctccttgagagtaaaaacccgcctgggtcgccatcaccctaggcttggtgcagggcctgccaggcgctcnaatttgttgaa

SEQ ID NO: 79 - DNA, 667 bp, Homo sapiens
  misc_feature (635)..(635): n is a, c, g, or t
ggtgactgacatggagcctggatgactatcgtgtacttgggaaagtctctttgctctatttgctgacatgcttcctgttgtggtctggccaatgccaaatgtactcgaatgatgttaagggctctgtaaaacttcatacctctttggccatttgtatgcatgatgtttggtttttaaacatggtataatgaattgtgtacttctgtcagaagaaagcagatgtactaatctccaattaaaaaattttttaacatgtaagaattttgtactttgaacaacaagattacagaaagtacctgtggtttttggaaaacatttctagcttggggaatgtgacaacattccccagtgtggtaaaattggggtaaaatgtggtaaaatgtgatacgcacaaaccctttgaaaatagcaaaacaaacatgccctttttctaaaattgataaatcctaaagaggaaagaaagagctgggacaataaacactggctctggaatctggaatgttaagtccaggccagcagtgacaaaagttattgtatgactctgaacagagaacactgccatgaaaagctctggtatagaaacatggtcattcagagctgtgaatatagctctagggtgctctgatcgtctgganatagctgacacatgaagtatggagggggagag

SEQ ID NO: 80 - DNA, 200 bp, Homo sapiens
  misc_feature (114)..(114): n is a, c, g, or t
ggtcacccagcaggaatagaccggcctggcatgctctgcagtttccggatccctggtgcctggtcctgtgcctggtccctgaatatccaagcaaataactgcttcagtacaggnttgtctcggcgggtcctgttgaccaacgtggngacgggacaccggcagtcctttgggaccaacagcgatgtcttggcccagcagtt

SEQ ID NO: 81 - DNA, 625 bp, Homo sapiens
  misc_feature (621)..(621): n is a, c, g, or t
gggaggcttagtttacaacgtactttgtacctcctgccatttaaattattttgccactctgggcttctgaaaacacaaagggagatacagccctccaactatgagccttgcagagagagacaaaagggccagaggtcctgggatggaatactagagcccccaccttctcatgctttggaaacctgccagagcacttaccctgctggaagattaatgaagactccattctgtgtttcataacagtagcgttgttttctccatcaaaaacagagggtcacagaaatgtgctgaaaagatgtggtccagcaaggttctgtagtcccacctcagtgcctgtgggctgtgtttttcactgtaacaagcactttggtaaactagaaagaagaaaggattctcaggatgtgttcaaagctaagagagttttcattttaaaaagaaacatacatactcactccatggctttcctcatcctcctctcttttttcttcttggtgggaagctggatgcggggcatgcatcattctgtatggacaaatgtgttatgtcccccatactcttgtgacaactccccagcactgtcctgcaatacatgctggcttggtaaaaactcctgggatctgngaag

SEQ ID NO: 82 - DNA, 300 bp, Homo sapiens
tgctagactacccctcttcatcagcctgctggaaagagcagacagcgtaacagcagcctatgccaaacagcactaaggatgtcagtgtacgagacggtgcgaagcccagaggaggcggcctgggatcctctgggccatccctggccctgtagcacagacacatacaagatccttggacttgtcacactccttttcacttgatccctctctggcctgggtcacatttatgacatcccccctctgcaacctttctctctgggggctgtaccaaaggcatggtacctcagtctggtatccaga

SEQ ID NO: 83 - DNA, 544 bp, Homo sapiens
  misc_feature (509)..(509): n is a, c, g, or t
gggtggacgctgggacgcgtttgtagctccggccccgccgttccgacccccgccgccgtcgccgccatgacggggctagcactgctctactccggggtcttcgtggccttctgggcctgcgcgctggccgtgggagtctgctacaccatttttgatttgggcttccgctttgatgtggcatggttcctgacggagacttcgcccttcatgtggtccaacctgggcattggcctagctatctccctgtctgtggttggggcagcctggggcatctatattaccggctcctccatcattggtggaggagtgaaggcccccaggatcaagaccaagaacctggtcagcatcatcttctgtgaggctgtggccatctacggcatcatcatggcaattgtcattagcaacatggctgagcctttcagtgccacagaccccaaggccatcggccatcggaactaccatgcaggctactccatgtttggggctggcctcaccgtaggcctgtctanctcttctgtggagtctgcgtgggcatcgtgggcag

SEQ ID NO: 84 - DNA, 344 bp, Homo sapiens
ggctttatagaattatatatgtatattctacttcttgatcaaagaacgtagtcgggtattggtttagaagttcaaagtgacaatgtatagggctttcacggttaatggacttgttaccaaaccttaaggatatacagccgaagattgtctgaggttgctggctaactttatttttcactgagttactctgcctttttgacgtttttattctttgtgtgtcagagttcagagctcaggagccaaaatatttttatacatatatagatatatatccatagcctggtagatttatatgcaatgcactgcatccatgcattctgatagcatttcattaattttgattc

SEQ ID NO: 85 - DNA, 360 bp, Homo sapiens
gcagatgtagaaattcatcagtgcctattggtcttccagaattttccatcttccgtatctcccaggcatgagactaccaagtttgtttgttttctttccaatttgggaatttatacttcagtatggtttcaacgcagttatgtttccagagaacatctagaagtggctggaaaccagaagctggggattccagggaccccacttagtgctctatttcctttataggttttatttctggtcatagagagagaaggacctttgactttttcttcgttgaggcttctgaggaggaaaaacaaacctaaaatagaaatacagtcagcctttcaaatccatgggttctgtgtccgtggatcaacc

SEQ ID NO: 86 - DNA, 526 bp, Homo sapiens
  misc_feature (384)..(384): n is a, c, g, or t
ggacgacttcaactgcaacgtctgggatgcactcaaagccgaccgggcaggtgtcttggctgggcatgacaaccgcgtcagctgcctggaactaacgccagtagcatgtggatgccatggagactggaagaccattccaacttggacgcgttaccatgagagcatatcctatccaaccgtactaacgtggacaccctacacctcccctcagaacttcaaaagggcaagatcttttttccttcacttattgctgaaaccaagagcacaattcccattgagagaaagatctctgtgctgtaaactaaaacaaattgtgcattccttccggggccatcgtctttgttttcttttttgtcttgaatgaattttaaaaggaaatatatnataaaaatgttaaccagaaggtaaacttgagtgtaattgtcagacagacacacttttccaccagtgtatttgaatttnaaccagtgaccctgttttgtggcattcatgcaaacatgctganggcttgttcatctggtcatcg

SEQ ID NO: 87 - DNA, 457 bp, Homo sapiens
aaggactctacccaagaagagctttgaacagatcagtgccaatataactaagtttaacattcagggccttgtcatcattgggggctttgaggcttacacagggggcctggaactgatggagggcaggaagcagtttgatgagctctgcatcccatttgtggtcattcctgctacagtctccaacaatgtccctggctcagacttcagcgttggggctgacacagcactcaatactatctgcacaacctgtgaccgcatcaagcagtcagcagctggcaccaagcgtcgggtgtttatcattgagactatgggtggctactgtggctacctggctaccatggctggactggcagctggggccgatgctgcctacatttttgaggagcccttcaccattcgagacctgcaggcaaatgttgaacatctggtgcaaagatgaaacaactgtgaaagggct

SEQ ID NO: 88 - DNA, 490 bp, Homo sapiens
  misc_feature (152)..(152): n is a, c, g, or t
gattcagcccccctaacccaccttgaacattgaccaccacacactccctggcacgctcttctcaccctcaacttctgccacctctccctgggcaatactgccagcctttccctaatctcagagctgcagccctgccctgtcactcacctcanacctgccatgtccctggggctactgaaattccnggcagtgggtgaagaggacgaggaggatgaggagggggagagcctggactctgtgaaggcactgacngccaagctgcagctgcagactcggcggccctcatatctggagtggacagcccaggtccagagccaggcctggcgcagggcccaagccaaacctggaccagggggacctggggacatctgtggtttcgactcaatggactccgcccttgagtggctcccacgggagctgcgggagatgcaggcgcaggacaggcagcggcngggcagctgctgcggctgcgggcccagctgcaccgacg

SEQ ID NO: 89 - DNA, 474 bp, Homo sapiens
  misc_feature (473)..(473): n is a, c, g, or t
gggaaaatatccctcttaatgatttcgtagttcagtttactgaatgattaccacctgtaattcctctttggattgtgtagactcaacatgagacattcctttctgctttctggagggcaccaggggcctttctctttgataaattttttttgtctgttgacaaaaacaaaaatcttttttcaaatgtagtgctggtgaaaaggtagggctgagtgattaccttagccacagggtggctgagcaggaactttagaagaaaatcctgagctttcctgtccattcccagcatccagctcctattctagtgcctcttccctgcagggcagggaccccttgggaaatcgaggaggtgggacgggctgggccctgtgtcccaggtttcacagggctcagggttatgctcccgcttgaatctggacgtgaatctggtaaaatatcaagtacctgtggaactccctgatctataccctctnc

SEQ ID NO: 90 - DNA, 546 bp, Homo sapiens
ggcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggactattctgtctctccttcttccccctggcctgttctgtctctgagctctgtgtcctccgttcattccatggctgggagtcactgatgctgcctctgccttctgatgctggactggccttgcttctaaagtatgcttctccacagctgtggctgcagga

SEQ ID NO: 91 - DNA, 424 bp, Homo sapiens
  misc_feature (390)..(390): n is a, c, g, or t
gcgacccgggtagtcttacgaccctggtgccctgggctgccgccctgctcctcgctctgcgcgtggaaagggctctggcgctacccgagatatgcacccaatgtccagggagcgtgcaaaatttgtcaaaagtggccttttattgtaaaacgacacgagagctaatgctgcatgcccgttgctgcctgaatcagaagggcaccatcttggggctggatctccagaactgttctctggaggaccctggtccaaactttcatcaggcacataccactgtcatcatagacctgcaagcaaaccccctcaaaggtgacttggccaacaccttccgtggctttactcagctccagactctgatactgccacaacatgtcaactgtcctggaggantaatgcctggaatactatcacctcttatatagac

SEQ ID NO: 92 - DNA, 387 bp, Homo sapiens
gggcgccaagccgcacaagtgccccatctgcgccaagtgcttcacgcagagctcggcgctagtcacccaccagcgcacccacactggggtcaagccctatccgtgccccgagtgcggcaagtgcttcagccagcgttccaacctcatcgcgcacaaccgcacacacacaggcgagaagccctaccactgcctcgactgcggcaagagcttcagccacagctcgcacctcaccgcgcaccagcgcacccaccgtggcgtgcggccctacgcctgcccgttgtgtggcaagagcttcagccggcgctccaacctgcaccggcacgagaagatccacaccaccgggcccaaggccctggccatgctgatgctggggccggcggcggcggg

SEQ ID NO: 93 - DNA, 236 bp, Homo sapiens
  misc_feature (213)..(213): n is a, c, g, or t
ggcggcccgggaggggggcgcagcacgcgccgcgcagccatggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgaaaccttcaccgagtggacnttccgccaaaaggtcactgagga

SEQ ID NO: 94 - DNA, 456 bp, Homo sapiens
  misc_feature (389)..(389): n is a, c, g, or t
gccaccagcatttcccttactctgaagttccggcattcacatcattcatgttttcttttgtcttttagctaaaggaaaagcattggcgatttgtctgattctggttttgagttactctttgttcagtaatgcactttattttattgtccaaagagagtcagagctaagcatacaggcttgggggtgagccctgctgtgagagttcaggccctgggaggctcagccacctcctcttgtgggaaggaggtctcagccccacctcgcatcttcacctgcccttggtgtggacacaccctctcatgctaccagcaccataatccagtgggggtgactgggtgcacacctgcccaggtgaacacagcggctgccagtctcctggtcccgagagnaggtggggcctggccctggctccctcaaccagctgctcctggnacacagggctcctgcttcggtctg

SEQ ID NO: 95 - DNA, 499 bp, Homo sapiens
  misc_feature (146)..(146): n is a, c, g, or t
ttgactcctgagttcaagagatcctcctgccttagcctcccaaagtgctgggattactagcataagcaccacacctggctgaacatcagatattttaaagtgtagcccagagtctagttggtaaatgttacttatattgggactantattttctaatgtttctgggatatgctccctataaaatgaaatgttttcctggaacaatgctaattaggattgcattcctggccnaagacatgacntcngataagtaatagacatcaccagcaatgaattttgaaaagcaaaaaatactattaacctatataaccatttaaaatttaaaaaattatgttccaagtacctctattttctgagctagaagaaaccttanaggccatctgatgatagattcccagagctggatggatccctagggactacctaatctgactttctgtccactgcatggattcttctaaaaagtatgtcacttatttccctaccttattatgactaa

SEQ ID NO: 96 - DNA, 410 bp, Homo sapiens
  misc_feature (250)..(250): n is a, c, g, or t
gaaagatccaatcagatgaacaatattcttcgaataattgctgatttacaagtttcctgcagctatgatcacctcaccaccctgccccatgtgcagaagtacctgaagtccgtacgctacattgaagagctccagaagtttgtggaagacgacaactacaaactgtcgctcagaatcgaaccaggaagcagctctccaagactagtctccttccaaggaagatcttgcaggtccctctgctggctccggnttctgcgaggttcagccggaggcccacctgtcctgacacatctgttgctggcagcctccccacacctccagtccccagacacaggaagagccacagcctaggcaacaatatgatgtgtcagttgagtgtagttganagtaaaagtgcgacattcccatcg

SEQ ID NO: 97 - DNA, 321 bp, Homo sapiens
gttcaaatctacagtacagtgcctcacatgtcacaaaaagtctaggacatttgaggccttcatgtatttgtctctaccactagcatccacaagtaaatgtacattacaggattgccttagattattttccaaagaagaaaaactcacagataacaacagattttactgcagtcattgcagagctcgacgggattctctaaaaaagatagaaatctggaagttaccacctgtgcttttagtgcatctgaaacgtttttcctacgatggcaggtggaaacaaaattacagacatctgtggacttcccgttagaaatcttgact

SEQ ID NO: 98 - DNA, 439 bp, Homo sapiens
gcttggaagcacaggcgctgacagccgtcccagcccttctgtctgcgggcctgaaccaaacggtgccatggggaactgtctgcacagggcggagctctccccctcaactgagaactcaagtcagctggacttcgaagatgtatggaattcttcctatggtgtgaatgattccttcccagatggagactatggtgccaacctggaagcagctgccccctgccactcctgtaacctgctggatgactctgcactgcccttcttcatcctcaccagtgtcctgggtatcctagctagcagcactgtcctcttcatgcttttcagacctctcttccgctggcagctctgccctggctggcctgtcctggcacagctggctgtgggcaggccctcttcagcattgtggtgcccgtcttggccccagggctaggtagcactcgca

SEQ ID NO: 99 - DNA, 626 bp, Homo sapiens
gcccttcatccctacagtactgatggcctcatcttccccttcaacccccagggacacactcaggacaccaacaccttctttctgtgccggacactgcgctccctaggggtccaggtttgccgagtctcagttgtacctgatgaggtagccaccattgcagctgaggtcacttctttctccaaccgcttcacccatgtcctcacagcagggggcatcggccccactcatgatgatgtgacctttgaggcagtggcacaggcctttggagatgagctgaagccacaccccaagttggaagcagccaccaaagccctaggaggggaaggctgggagaagctatcattggtgccctcctctgcccgcctgcattatggcacagatccttgcactggtcaacctttcagattccctctggtctccgtccgaaacgtctacctcttcccaggcattccagagctgctgcggcgggtgctggaggggatgaaagggactattccaaaacccagctgttcagttccactcaagggagctatatgtggctgctgatgaagcctccatcgcccccatcctggctgaggccaggccacttggacgtaggttggcctgggttctacctgatggggcag

SEQ ID NO: 100 - DNA, 551 bp, Homo sapiens
  misc_feature (539)..(539): n is a, c, g, or t
ggtgctcacctggctggagccgaacactctttactgcgtacacgtggagtccttcgtcccagggccccctcgccgtgctcagccttctgagaagcagtgtgccaggactttgaaagatcaatcatcagagttcaaggctaaaatcatcttctggtatgttttgcccatatctattaccgtgtttcttttttctgtgatgggctattccatctaccgatatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcatcaggatatgagtttactgggaaaaagcagtgatgtatccagccttaatgatcctcagcccagcgggaacctgaggccccctcaggaggaagaggaggtgaacatttagggtatgcttcgcatttgatggaatttttgtgactctgaagaaacacggaagtacttctctcaccagnagagtcctcaca

SEQ ID NO: 101 - DNA, 545 bp, Homo sapiens
ggctctgtctccaccgcagggcccaaggtgaaagtcctccccttgccggaggccagctggcagggcctttcgtggctggaagtggccagtttggttccggtgctgacccctaggccccagcgcagctgcctcccgtgctgtctgtctccccctctctgtttatgtctgcgctgtgtctcacactcagagcctccttgcttctgttaggttcccatctctccttctgcctcactctgggcctcttctttctacttgtacatttccacctctctaggcctctgctctcactgtccctctctgtctgtctctcagcctttatctctgggttttgatcccccactccaggctctgggccccttcttcccccttccctcaaacctggctgctataggcagcagaaccctgaccactgagtattacagccccaggggagggaggaaacccaggcagggaggatgggggcagctctcttctctcccaggaccaggctgtggatcacgggcctgccagctgatggaaagggagggggatctaccttccagccc

SEQ ID NO: 102 - DNA, 592 bp, Homo sapiens
  misc_feature (540)..(540): n is a, c, g, or t
atgggccaaaatggtgggaaattagcttggtaaggcagctcatgaagatcttcacaacagaagagtttggcaattattgattgagcaactggacaccctgactgaactcttttgccaatagttttatggtggaaaaacaaaacctcctcagctcctctttcactccttctcttctgcaaggaaaacgattttcagactagaaaggacaaggtgaacaccagagagaaactcctgaaggcagtctagttcaagagagccgtaagctgccttgagtaactgcatctcattgtctgagtcagtagcatcccagggcacttaaggacttgcagctcaaattgcaggacaactggtggtggccctgaaaaccccatgaagaagggaagagatacgtgctgcagcctagagatgggtaaacatggccccactttgacagatgggaaaaggtaaactccacaaattatagatcaatacatttgatattgattgagggcaagatctaggacaaatgattaaaggatggttggtgagcactacaaaggnagcgacgatcactcagtcccccatggatcaggaaaacaatcatgccccctgg

SEQ ID NO: 103 - DNA, 344 bp, Homo sapiens
gcttggagaggctcttcctgggtgtgtctagttggccctcttttccctcctcttcttaggatactttttatttctttgctatcagttgtaacgtttcctctttcatttctgatttttgttattcgaatcttctttcttttttcttagtctggttaagaatttgctgaatttacctttaaaaaaccgaactttccattgtggaagacattgtggcgattcctcaaggatctagaactagaaataccatttgacccagcaatcccattactgggtatatagcaaaaggattataaatcatgctgctataaagacacatgcacacacgtatgtctattgtgaaaaag

SEQ ID NO: 104 - DNA, 559 bp, Homo sapiens
  misc_feature (553)..(553): n is a, c, g, or t
gggttgtctgaaggccgaggccaagatggcggtgctgtcagctcctggcctgcgcggcttccggatccttggtctgcgctccagcgtgggcctggctgtgcaggcacgaggtgtccatcagagcgtggccaccgatggcccaagcagcacccagcctgccctgccaaaggccagagccgtggctcccaaacccagcagccggggcgagtatgtggtggccaagctggatgacctcgtcaactgggcccgccggagttctctgtggcccatgaccttcggcctggcctgctgcgccgtggagatgatgcacatggcagcaccccgctacgacatggaccgctttggcgtggtcttccgcgccagcccgcgccagtccgacgtcatgatcgtggccggcacactcaccaacaagatggccccagcgcttcgcaaggtctacgaccagatgccggagccgcgctacgtggtctccatggggagctgcgccaacggaggaggctactaccactattcctactcggtggtgaggggctgcgacgcatcgtgcccgtgnaatcta

SEQ ID NO: 105 - DNA, 437 bp, Homo sapiens
  misc_feature (38)..(38): n is a, c, g, or t
aagtgccctgtgtgcctctctgtgcctctgggagttgngagggaggaatgaggcatgaggtgatacctggggattagtggcctgctgggacttctgctggagcctcttcataccagagccctgtagtgaggtatcttactgcgggggaaggaaggaggcttgcctgcagcattctcttgctgggaaagacagaagggcacgtggtaagaagagggtcattggatcgaagctcatgtgcaccacagctgtttccccatcctccagtggatggcacaggaccccagggttagcacactggcacttggganctataaggtgaggatctgggnaagttcttggggactggaaggccctcctctgctctagcctggccaacatggtgaaaaccctgtctactaaaaataaaaaaattaactgggcgtggtggtgggcgcctg

SEQ ID NO: 106 - DNA, 440 bp, Homo sapiens
gggatgggtgaatcgcttgagcccgggagttcgagactagcctgggcaacatggcggaactccgtctctgcaaaaaatacaaaaattcgtccagcatggtggagcatgcccgtagtcccagctgcttgggaggctcgggtgggaggatcgcctgagtcggggaggtggcggtgagcggagatcacgccgctgcattccagcctgggtgacagaacgggatctcgctgtcacccgggctggagagcagtggcacaatcacggctcactgcggcctccgccttccgggctcaggtgatcctcctgcctcggcctcccgggtagctgggactacagtgctctcctatcaaatctagaattggaagattgatggacagcaactgaggtgctaccaggataccaaaagtctgctggataaacacatctctattctatattatgga

SEQ ID NO: 107 - DNA, 551 bp, Homo sapiens; identical to SEQ ID NO: 18
  misc_feature (549)..(549): n is a, c, g, or t

SEQ ID NO: 108 - DNA, 538 bp, Homo sapiens
  misc_feature (492)..(492): n is a, c, g, or t
ggtattgagaaattcgtggaagaaatgtcagcaggaagtaaaaattcaccgagaagtgtgtgtgtgttcgctgcttccacacattaatggcatgattttttatgcaaaaagaaaagaaaagaaaaccacccacattttaatttagcatgagccaatttacagagcatggaaatcactttcatttccggattggcgcgtgtgcaattagcaatgcagtgtatatacaagaagccatgctgttaacagtttatctcaagtaatttggtgtcatttacaaaaggaagaaattcctgccatcagaagggacagaaggagatggaatgatcaaatcacagagaaacactaaaattactaaatctacaacagctcgccttatttttcttggacccaaactgtcagggtataaacactatttgtttcttttttaaaacatcgatagttttgctgtaaataatacactgtataaattctaacagaaaatagaataaatgntgataaggcactgcctcttaaacacaacagaatattgcaatgcatt

SEQ ID NO: 109 - DNA, 460 bp, Homo sapiens
gagatggagaaggaggagacaacccgggagctgctgctgcccaactggcagggtagtggctcccacgggctgaccatcgcccagagggacgacggcgtctttgtgcaggaggtgacgcagaactcccctgcggcccgcactggggtggtcaaggagggggaccagattgtgggtgccaccatctactttgacaacctgcagtcgggtgaggtgacccagctgctgaacaccatggggcaccacacggtgggcctgaagctgcaccgcaagggggaccgctctcccgagcctggccagacctggacccgtgaagtcttcagctcctgcagctctgaagtggttctgaacacaccacagccatcagcactgggaatgcaaagaccagaacaaacagaaggaagccagcagcccagccgggggcagtttcagtctccaccccaaatgcaggactgtaaaagcg

SEQ ID NO: 110 - DNA, 566 bp, Homo sapiens
  misc_feature (493)..(493): n is a, c, g, or t
gcttctggattaatagggccagatcatggcacacacagcttctcaagatcacatccctgctgatctctctggccctcctgatcactgctcccccacttcacaccctgtcctgcagccaggccctactgcttgctgttcacaaattgttccatgtgatgtcacttccttagctctggtctggctggtccctgaacctgacattcccttcctccccttgtgaccctctaatgaacgccaactttcatctttgaagggttagcttctattacctccttcatgaagccttctggataacattggccatatccttctaggcatcttctaagaatagtgtaataggctgaatagtggcccccaggagatacattcactgggaatctccacgtgtgacattatttggaaaaagtttttgcagatgaaatcaaggacctggaaatgagatcaacctgtagtagggtgggccctaaattcaatgactagctcttaaaaagangggaaatagaccacagagctgaagccatgggaagatggaggcagagataaagcaatgcatctatgagcaaana

SEQ ID NO: 111 - DNA, 444 bp, Homo sapiens
  misc_feature (440)..(440): n is a, c, g, or t
ggatttttgtatttttagtaaatgagttttcaccatgttggccaggctggtcttgaactcctgacctcaagtgagctgcctacctggcctcccaaagctctgggattataggtgtgagccactgtgccaggcctgaggttgggagaaattttgtccatttctttagaaccaaaattggcaaccagagagtatttggatgttacacaaaatatttagtttccctttctagcctaaattgggttgtttatagcacccgtctctccatttgagaaaaatggttaggatgctggtgcagggatgaggccttattaaaacagagaaggtacccaaatggtggttcaacacaggctggctaataagctgtgaggtgacttaccaaaaacaacaacaacaaaacccttgaacttctgctttcaacttctactttatgctagatattnggcc

SEQ ID NO: 112 - DNA, 377 bp, Homo sapiens
  misc_feature (23)..(23): n is a, c, g, or t
cgcggcagtcctgatggcccggnatgggttaccgctgctgcccctgctgtcgctcctggtcggcgcgtgcctcaagctaggaaatggacaggctactagcatggtccaactgcagggtgggagattcctgatggaaacaaattctccagacagnaaagatggtgaagggcctgtgcgggaggcgacagtgaaaccctttgccatcgacatatttcctgtcaccaacaaagatttcagggnattttgtcagggagaaaaagtatcggacagaagctgagatgtttgnatggagcttttgtctttggaggactttgtctctgatgagctgaaaaacaaagccacccagccnatgaagtctgtactctggtggcttccag

SEQ ID NO: 113 - DNA, 278 bp, Homo sapiens
ctgaatagctctctaggtgcaaagagaagagtaaggcatggtcccagatcagtggaacttaggttttaagaatgttcacttactatacactctgtgacgaagcctaaaataaacttagcctaccatctctataggttttataaaatttgcaaaagtaatccttttctcagtaaattcaagtaatggaaatgtatatgaaaaagtaaacttctttgttcttcaccagtcccactgcgtggagctaactgccataaacagtttgctttatttggtcccgg

SEQ ID NO: 114 - DNA, 391 bp, Homo sapiens
  misc_feature (287)..(287): n is a, c, g, or t
ttacaaaaaacagtgaagtaagagtactcctgaagactaaaatagagaggctggggtttgagccattttactgagtagcttagctggaacctgatatcagaagtagcctttaacaaaaagcctcttggcaattgtatggtactaacaactagagtactgaagtgtaagttgaaaccaagttgcagggggaaatcaaaggtgaggtagcttatttgaaaccagcaaatgagacaggttggacagttttaaaatctcttctaacaaagaaactgcccgggtagcaaggnactagcgnttctcaaagcccnttctttttcagtgttctcattcaccttggccacccaagtatgtttaacaggncatgcattaaaaaataatacaaaaattttta

SEQ ID NO: 115 - DNA, 430 bp, Homo sapiens
  misc_feature (429)..(429): n is a, c, g, or t
ggggaaagccatttcgaaaacccatctatacaaactatatattttcatttctgctgctagctgccttgggcctcacaattttcattctgttttctgactttcaagttatataccgtggaatggagttgatcccaaccataacatcgtggagggttttaattttggtggtagccctcacccaattctgtgtggctttctttgcagaggattccatccttcaaaatcatgaactctggctgttgatcaaaagagaatttggattctactctaaaagtcaatataggacttggcaaaagaagctagcagaagactcaacctggcctcccataaacaggacagattattcaggtgatggcaaaaatggattctacatcaacggaggctatgaaagccatgaacagattccaaaagaaactcaattgggaggcna

SEQ ID NO: 116 - DNA, 361 bp, Homo sapiens
  misc_feature (297)..(297): n is a, c, g, or t
gcagttctgaaattctgtttggtcagttctagatggtacgtcatgtgaatgcaaccaagcactgtagttgaaattgtgttatgccagtactcatatgttgtcttaggtactacgcataatgttaatagctgagatgttaaagaatttgaagtctaaaatataaaagatgaatatacccatattaatcctatgttaagatgctctggaaataaaggccttattcccttacacatgcgatttttgtaagataatatatacacagtatattttaaatgtttgtgtgggtggtctgtgtanttactccccatacaacaaagctgacaaaatttttatttacacaatgtattctgcattttcaatg

SEQ ID NO: 117 - DNA, 483 bp, Homo sapiens
ggtctgatacctggccccgggatagtcaggacaggtgaatgttggcctggggtgtgacagccctgggagagccatgtgaaggaggcagctggcgccatcgctccggattagttggtttccataggaaaggcatgctttcagccagatgcttgccatctctagggattgggggattggctagcctgggaggatcagtctgtccaggtcagcgaggccccagataccagagcatcaagagtacaggaaatacagttaatgcagggcctctgtgtggctggatcctccgtctccatcagatcagctctgattgatctattcttgcacgatttcctctgaacacagggttccagagtacttaaacacaacattttttaaatcgtgatttcggcctatttccttgccaggcctgtttccccccaggaatgagatagtaggactggatgaggatgtcctgttatagttgctgtggagaagtcctctggt

SEQ ID NO: 118 - DNA, 317 bp, Homo sapiens
ggacgtctcctacctcctcacggctcttcccggcgctctcctggctcccttctgccccagctccgtctcggcggcggcgggcagttgcagtggtgcagaatggctgacctcagtcttgcagatgcattaacagaaccatctccagacattgagggagagataaagcgggacttcattgccacactagaggcagaggcctttgatgatgttgtgggagaaactgttggaaaaacagactatattcctctcctggatgttgatgagaaaaccgggaactcagagtcaaagaagaaaccgtgctcagaaactagccagat

SEQ ID NO: 119 - DNA, 429 bp, Homo sapiens
  misc_feature (386)..(386): n is a, c, g, or t
gacgatgatcagaaagctgtgaaagatgaactgtaatacgcaaagccagacccgggcgctgccgagacccctcgggggctgcacacccagcagcagcgcacgcctccgaagcctgcggcctcgcttgaaggagggcgtcgccggaaacccagggaacctctctgaagtgacacctcacccctacacaccgtccgttcacccccgtctcttccttctgcttttcggtttttggaaagggatccatctccaggcagcccaccctggtggggcttgtttcctgaaaccatgatgtactttttcatacatgagtctgtccagagtgcttgctaccgtgttcggagtctcgctgcctccctcccgcgggaggtttctcctctttttgaaantccgtctgtgggattttaacatttttcgacatcagggtattgt

SEQ ID NO: 120 - DNA, 440 bp, Homo sapiens
  misc_feature (439)..(439): n is a, c, g, or t
caaataaatcttaaaattcagttctcggccatgacaggacaggaggtcagacatacctccctgtacctccctccctcttatggtttagacccaacaactgagcagcattaatgttaaaatagagatcatgagactgacagaacagactctttgtggcaataagacctcaaattataaacagggcctagggccatgccaggcgagggttaagtcttgtaccctactcttaaagaattaattagattctaactaccacatgggttttatttttctctagcagccaagcaagcactggctgtgagagaagcaagattaaaacaattacaactcacccagttcacagacgctgagtaactgatctcctgccccactaacttaacgacagctttctctggacaagggactgatttcagtaactttctcctgatagagacatccnc

SEQ ID NO: 121 - DNA, 406 bp, Homo sapiens
  misc_feature (383)..(383): n is a, c, g, or t
cccaggccatccctctgttgccatcagcttgattggcttccccgagggccagcagggctgggggctccggggagcagcaggaagcactcccagccaccagtgcctgtcgcctctttcccctttgcccctgcttcatcccagctctgtgtgtggaggacaaagcttcttcctgcgtggctccaggaaaagatgtggctcacgtaggtggcacctgccaatagctttgtcaatcacagccccataggaacgtctggaattgcttgggagttggggagaactgtcaagaagagtgaagagagtgccaaagcggagatctgttcacctgggggccatggaggggggacccactaaagatcaagatcaaagattctcccatctcacanacaagaaactgaggccagaggga

SEQ ID NO: 122 - DNA, 478 bp, Homo sapiens
  misc_feature (477)..(477): n is a, c, g, or t
cgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggacaccctccctggccttctgcagcagggttctgggatgttttcagtggtcaaaatactctgtttaaagcagggctcaaaaana

SEQ ID NO: 123 - DNA, 331 bp, Homo sapiens
  misc_feature (210)..(210): n is a, c, g, or t
gccatgttgttgaggctggtcttgaactcctgggttcaaacgaccttcccgcctcagcctccgaaagtgctgggagtacaggtgttagccactgcgcctggcctcattgtactccttaacacaagaagaattcaacaatgataagtagttgtttataaggaagcaggatcattaccaaaataaatcctgctaaaacaacaggaatcatgntttaaagcctagtttgctaatttttgctagtaggataanagtgatcgtaatatctcgaacattacatagacacttaaaacctttagttgnattccatcaaaaatctgttcataccccacgt

SEQ ID NO: 124 - DNA, 256 bp, Homo sapiens
  misc_feature (254)..(254): n is a, c, g, or t
tgaaatgtataagtccataaaatagaaaagaacaagtgaattgttgctatttaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttcccccaaaaangg

SEQ ID NO: 125 - DNA, 336 bp, Homo sapiens
  misc_feature (321)..(321): n is a, c, g, or t
tggacaccgtgccccgctgaccccactggcccctggcctgccggacacagcaccgtgccaaccccacccagctccaggcccaccggatgctgtgcctggcctccgatgcctggcctgccagacactgcgccccgcctgacctgggggagccgaccaattagtcactgctgctactgcccctctccgaaagaagacacagaaccaacaaaaccgcattcagtgcacctgcctcagctacctaatgattccgcgcggagacctcctgacaacgtctcttcaagcatcctcagaagcctcgactgagctttaacagcagagcanatgccgcaggcgcgg

SEQ ID NO: 126 - DNA, 491 bp, Homo sapiens
  misc_feature (485)..(485): n is a, c, g, or t
gaagaattcttgctaacagaaattaacttctaagtaaagcagtggaaataattgaacattaatgagccaattacaatcataagtactttttaggggatactttagtgatttttgtcctctaatgaaatgaaaaccaacagactggattgagaaggctgctttttttcttgctgttgtggttgttttgctcttcctctgtagtctggctcaaaggaaacaaagcaggatgatgactgaatcttggattatggggtgaagaggagtacagactaggttccagttttctcctaacacgtgccaagcccaggagcagttcttccctatggatacagattttcttttgtccttgtccattaccccaagactttcttctagatatatctctcactatccgttattcaaccttagctctgctttctattactttttaggctttagtatattatctaaagtttggcttttgatgtggatgatgtgagcttcangtgtct

SEQ ID NO: 127 - DNA, 534 bp, Homo sapiens
  misc_feature (498)..(498): n is a, c, g, or t
gcacaggtagcaagaggtacttttacctggctccaaatctgtgttctttccactgacaaatgagatatgggatatggtgcatctttacagtactataataagtattggcgtataacattattttcaaggaactccaagggccacaggagctgacaggtttttcaattaatattcccaacatgaatgagatgcctcattcctcagtttcctcacgtgtactataaggctagtacctgctttgttggggtatggttggctcgtgtgcattaagtcaacaaatccctagttgatagggtttggctgtgtccccatccaaatctcatcttgaattgttcccgtaatccccacgtgacatgggagggacccagtgggaggtaatttaatcctcggggcggctacctccgtgctgttctcgtgatagcgagtgagttctcacgagatctgatggttttataaggggcttttccctcttgttcggcacttcctgctgccacgtgnagaaggggtggttgtttccccatctgccatgattat

SEQ ID NO: 128 - DNA, 154 bp, Homo sapiens
  misc_feature (149)..(149): n is a, c, g, or t
taggggcgtctggcctttttcattcctgggacccacagctcgaggtctggtttccatgccctatcccttacgatgagcgccctattttgttccttctgtctatctctgttgctgagatcagggagcccacgttcacagcagctctttcnagcag

SEQ ID NO: 129 - DNA, 352 bp, Homo sapiens
  misc_feature (2)..(2): n is a, c, g, or t
cngaggattgttcccttccatttccccaagtacttatcacatggctgctcctatttggcccagcccagtgacgttctcttttataaaggactctctaagggctgttttccactgtgacaagtcttggtttgattggtcacctcccagtgagtggacatcattcagccaccaggacctgtcacatccctttttcacttaactttggatctantgaaatcctgcacagttcacattctggacatttaacacgtattcttttttataagaactgatagtacctattatttcatacacaaacacacgcacacgtacagatacagccggccttctatatccgtgggttccacatcng

SEQ ID NO: 130 - DNA, 402 bp, Homo sapiens
  misc_feature (372)..(372): n is a, c, g, or t
gaaattttctactaaaaagcatttcaacgttttccaatgtgttctctttacaaaggaaagtaatggtgagaggcttgttcaccactgaaaagtttggagaactaaaacataattcaaattttataaggatggagatattctgaaaagtgggaagaggcaagcacttcaataaggggcagtatctctgggagatggaaacgaacatcctcaggaaaaaaaagagagggggttaaacaagaactcaagggaaaattgaggagcaccacttgcattctaattcatttctcttgaaagaattcattaatttactcaggcgcatgtactagtgaccccagaacaaatgagtttctgccctaaggaattcgttactgnatggaaaaattcagggggataaagcagctg

SEQ ID NO: 131 - DNA, 531 bp, Homo sapiens
  misc_feature (451)..(451): n is a, c, g, or t
gcgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttagnacaaaaagcaagtcccccatgagtgtccacttctttgccctgccctctcagcttgngagaaacacaggagccttctatag

SEQ ID NO: 132 - DNA, 538 bp, Homo sapiens
  misc_feature (516)..(516): n is a, c, g, or t
ggccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagggccaaggcgaaaagactgcaggaggcagaactggaaaagctgaaaatggctgcaaaacctatgctgccctccagcttcagtctccctttccccatcagctcgcccctgcaggcagcgtccatatatggagcatcctacccgttccatagacctgtgcttcccatcccgcctgtgggactctatgccacgccagtgggatatggcatgtaccacctgtcctaaggaagaccagatcaatagactccatgatggatgcttgtttcaaagggtttcctctccctctcccgaaggcagtaccagccagtactcctgctctgctancctgcgtgcaccaccctagcgg

SEQ ID NO: 133 - DNA, 524 bp, Homo sapiens
  misc_feature (453)..(453): n is a, c, g, or t
gatagcctatgaccaactgcagcaagacaggaggtcagctcgcctataatggtgcttaaagtgtgattgatgtaattttctgtactcaccatttgaagttagttaaggagaactttatttttttaaaaaaagtaaatggcaaccactagtgtgctcatcctgaactgttactccaaatccactccgtttttaaagcaaaattatcttgtgattttaagaaaagagttttctatttatttaagaaagtaacaatgcagtctgcaagctttcagtagttttctagtgctatattcatcctgtaaaactcttactacgtaaccagtaatcacaaggaaagtgtcccctttgcatatttctttaaaattctttctttggaaagtatgatgttgataattaacttacccttatctgccaaaaccagagcaaaatgctaatacgttattgctaatcagnggtctcaaatcgatttgcctccctttgcctcgtctgagggctgtaagcctgaagatagtggcaagcacnag

SEQ ID NO: 134 - DNA, 413 bp, Homo sapiens
  misc_feature (407)..(407): n is a, c, g, or t
gaaattaattatcattttttaaaaaaattttatttttattttttcgagacagagtctcactctgttaaccaggctggagtgcagtggtgcgatctcagctcactgcaacctccgcctcccagattcaagtgagtttcctgccgcagcctcccaagtagctgggactacaggcacgcaccatcatgcctggctgatttttgtatttttagtagagatagggtttcactatgtttggcctcaaactcctgacctcaactgatccgcctgcctcggcctcccaaggggctgggattacaggtgtgagccaccgtgcccagccaaaattttcattttttgagacggagtctcactctgtcacctaggcgggagtacagtggcgagctcttgggttactgcaacctccgccntcctgg

SEQ ID NO: 135 - DNA, 511 bp, Homo sapiens
  misc_feature (506)..(506): n is a, c, g, or t
gggcgcccgggtgaggcacgggtgcgcaagcgggagttccggctggagacccgtgctctgggccggcgccttcaccatggcctcggcagagctggactacaccatcgagatcccggatcagccctgctggagccagagtgctaacgcggcttggctgtcaccttcaatgcatcacccgaagctgagagccccccttcgtcactcctagcttgcagcctcacaattccatttccccacccaaatacatccattttaatatgcacatcacaggcaggcagcacggtcacaaaggagccccaccagcagcagtccccacgtggtggaacggatggatgaccagccaccagcctcggcctcttggcttccagcagatggatacccagcagaggcctggcaccagagtgccaatgtctacgtgtcagctgcttgtacctcggaagccaccttccaggagagtggtggcccgtatccgcttctgtccacaccactcatactcctcaaagggntggtg

SEQ ID NO: 136 - DNA, 506 bp, Homo sapiens
  misc_feature (473)..(473): n is a, c, g, or t
gccgcaagtgcagagtcaaggcagggagaggccggctggagccaggccccctcgcacgcagcccccaaatcatggacgcacctgtggggagcaccacatctccacctgcggcctcacatctccccactcccctttttgtacgtttaactgtttctttgtacgtggtttacgtaactttaaactgtaacagccttaatggaagaccaaatggttttttatatgtgtatgtacaaagttttctattaacgctgcccgtctcccttataacctggacgtgagctgtcagagcagaagccactaggccactgcgcgtctgaggctcagaccctgctgtggttggcttggggtggccaatgggctgggaccctccatgagagttttggacacttgggtcacctgacccgtgcctctctgacacatgtctccggggggcagccacctggccaatgtgcatttttgcacatgctggaacntccatgggggtctgggctattggctggagccag

SEQ ID NO: 137 - DNA, 460 bp, Homo sapiens
  misc_feature (450)..(450): n is a, c, g, or t
gaccacttcaacgtgctcgccttcccctgcaaccagtttggccaacaggagcctgacagcaacaaggagattgagagctttgcccgccgcacctacagtgtctcattccccatgtttagcaagattgcagtcaccggtactggtgcccatcctgccttcaagtacctggcccagacttctgggaaggagcccacctggaacttctggaagtacctagtagccccagatggaaaggtggtaggggcttgggacccaactgtgtcagtggaggaggtcagaccccagatcacagcgctcgtgaggaagctcatcctactgaagcgagaagacttataaccaccgcgtctcctcctccaccacctcatcccgcccacctgtgtggggctgaccaatgcaaactcaaatggtgcttcaagggagagacccactgactctccttcctttactctnatgccattgg

SEQ ID NO: 138 - DNA, 453 bp, Homo sapiens
  misc_feature (450)..(450): n is a, c, g, or t
gaagtatttaactctttcactacagttcttgcaagtatgctatttaaattacttggccaggtataattgccagtcagtctctttatagtgagaaaatttattggttagtaatataaatattttaaactaaatatataaatctataatgttaaacatatgttcattaaaagcatagcactttgaaattaactatataaatagctcatatttacacttacagcttttcatttgatcaggtctgaaatctttagcacttaaggaaaatgactatgcataattatacctgaccatgaaaaaaataagtacctcaaatgcatgcatttgcactggtgattccaactgcacaaatctttgtgccatcttgtatataggtattttttacatgggttgacatgcacacaacaccattttcattcagtatgaaccttgaggctgctgccatttttcccntta

SEQ ID NO: 139 - DNA, 463 bp, Homo sapiens
  misc_feature (373)..(373): n is a, c, g, or t
gctacaaatgatccaccaaagcaaagccctgggtccacttcccctaaaccccctcatacattaagccgcaagcagtggcggaaccggcaaaagaataagagaagatgtaagaacaagtttcagccacctcaggtgccagaccaggccccagctgaggcccccacagagaagacagaggtgtctcctgttcccaggacagacagccatgaggctcgggcaggggctttgcgagcccgcatggcacagcggctggatggggcccgatttcgctacctcaatgaacagttgtactcagggcccagcagtgctgcacagcgtctcttccaggaagaccctgaggcttttcttctctaccaccgcggcttccagagcnagtgaagaagtggccactgcagccaggggacgcatcgccagggatcttgccagcggcctgcatccctaggggggctgactncgctggggg

SEQ ID NO: 140 - DNA, 370 bp, Homo sapiens
  misc_feature (360)..(360): n is a, c, g, or t
gaaactcccagctggtctcaagcaatcctcccaactcagcctcctgagtagctgggacaacaggtgcacaccaccactcaaggctaattttctttttatttttggtagagataaggtcttactatgttgcccaggctgatctcaaactcctggcctcaagtgatttttctgccttggcctcccaaagcactgggattataagtatggccactgttaccagccttagatcattttttttaatacagatagcttcccaactaaaaatattttattagaattctctgaattcagcaaagcaatgttattactgacccagtcttcacttattttcagctgacatggaagaacaaaattaccttnattttaaatt

SEQ ID NO: 141 - DNA, 337 bp, Homo sapiens
  misc_feature (321)..(321): n is a, c, g, or t
gcagaaacctgttttgatatgtgttctccatgagttaagtctgatttgtctttttatttcatgatgcatgtcttttttttcttttgtcaggataacgtcatatagcatcttgtttgtttttccttatctctatgtacatatctatctacttctgactgtagatgggtatatagatagatgccaagcttcttatgttctgggggtagtatgcatcattattgggtctctgccttaaaacacatcaaaattcattttagacaaaaaaacttctgctttgtctttggtcattagggagctctaatgtgtgtttgtggctccagntacatttggggttcat

SEQ ID NO: 142 - DNA, 410 bp, Homo sapiens
ggctcggcgcccgggtcgcggcccacctggacgcactgggccccctggtcccctacgtgccgccgccgctgctgccctctatgttctacgtgggcctgttcttcgtcaatgtgctgatcctgtactacgccttcctcatggagtacatcgtcctcaacgtgggcctcgtcttcctgcccgaggacatggaccaggcgctcgtggacctcggcgtgctctccgaccccggctcgggcctttacgatgctgactcggagctcgacgtctttgatgcgtacttggagtagggtctcgactgctgttcccctcttccctacacgatccgcaacccatgccctggaccagccgcccagatcatgccgccgcagctggttgggggcaccatctggacggggatggttccccaggag

SEQ ID NO: 143 - DNA, 441 bp, Homo sapiens
  misc_feature (162)..(162): n is a, c, g, or t
ggttaattatagctaagtgattatgtgcagtgcagcctgacatctagccacagtaaactcctgttttcttgggatccaaacaccctgtgttttaccattagttttgttagtattgttaactttctttacacaagtaaaccatttccactaccaaaggtttanaaactatggatgaacaaaatgaatctccctgttcattttgagcctacatagacatagagaccaaatatgtcaaaaccttattcaaataaactctcttcagagtgaatacccagggtcaggacagactcagcaaaccttctatatcacttaattctgcccagtgaaaatcagtgaccctgaggcactgccagattctaaagngcctttgngatcatcagagactgtctataaataatgcaaantcttgatctggatgtcagtgtatcaaatcttatcaaa

SEQ ID NO: 144 - DNA, 356 bp, Homo sapiens
  misc_feature (351)..(351): n is a, c, g, or t
gaaagatttcttgtttatttcctcttgcagtcatgcacatgatttgggtaaactgtgagatgagaaatggttttcagagtattagatggaattcacccccgttgaagtttataaatgtgttcaggggaagcgggaggaaagagttcactgcctaatcagttttgcatgtcatgaaaattaaattcctctccaggtgcagcttcagcctcatgcaacttaaagtgataacagttatttgattttttaaaaaatattattccaaaagaaaaccattttaggtcatctcccccaactctgtttgcttactgcttaataaatataaaaataaatctaaaaaaaaaaaaaaaaaanaaaaa

SEQ ID NO: 145 - DNA, 314 bp, Homo sapiens
  misc_feature (238)..(238): n is a, c, g, or t
gagttgcttgtagttttaaaaaaataataaagtatacccttctggtatatcatcaagagcttaagaatcttggctttcatatttaaaatgcttttggggagacatatattaaaattttagccaagatgatagacatgtctcaattatatatgtgtgtgtatgtttttaaagctaaaaacattacttttagatccctagaatgaaaatttttttctcatctatgcaattcccatatggntttttttaaatcatattttattcattttctccctttagcaattttcattttattnctcataattgaacagagacag

SEQ ID NO: 146 - DNA, 296 bp, Homo sapiens
  misc_feature (288)..(288): n is a, c, g, or t
gagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaacccngcataaat

SEQ ID NO: 147 - DNA, 502 bp, Homo sapiens
  misc_feature (474)..(474): n is a, c, g, or t
ggatttgtgttggatggctaagctctaaaataatgcaaatagccccaatctttaaatatagcggtgctaagttgaacaagtaacacaatacagaaaatgctgatatgaatacttagtaataatacaaaagttcctgctaaattatatatgtgtatcactgcctgactagaaaccccacttttcttttctaatccagcacaaaatcaaactctgattctacaaccagtctttttaaagggcaaaaatgactcaacacctttgttttgtatggaaaacttttttttttacactaactgcaaaactgctttaaaaaaaaggcttattcaggatagataagcatgtactccttttttaacttgctggaagacttgcccctccaaactagcacccccaaaagacaacttctttcagaaacggggtgttttacctaaacatagtagcttacatgttagccagcagtaggtcggcactagngttttccacggttatcacctttgacagg

SEQ ID NO: 148 - DNA, 489 bp, Homo sapiens
  misc_feature (406)..(406): n is a, c, g, or t
gaggactcacatctgtttcagtttttcctcagttgtatattgaccagtgttctttattgcaaaaacatatacccgatttagcagtgtcagcgtattttttcttctcatcctggagcgtattcaagatcttcccaacacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcagccactagaattatcaaatatgacgctntagcagagtaaatactctgaataagagaccggtactagctaactccnagagatcgttagcagcatcagtccccaaacacttag

SEQ ID NO: 149 - DNA, 404 bp, Homo sapiens
  misc_feature (387)..(387): n is a, c, g, or t
gcaaaaacaagttaaggtgaactgcacagctgaagtactttacccttcaacgggacaagaaactgcaccagaagtcaacttcacatttgaaggagaaactggaaagaatccagatgaagaagacaacacattttatcaaagacttaagtccatgaaggaaccgctagaagcacaaaatattccagacaattttggaaatgtatctccagaaatgacgctcgttctacatttagcctgggttgcctgtggttatataatatggcaaaattctactgaagacacatggtataaaatggtaaaaattcaaactgtcaagcaagtgcaaagaaatgatgactttattgaattagactacaccattctacttcataatatagaatctcaggngattttccctggcaaag

SEQ ID NO: 150 - DNA, 458 bp, Homo sapiens
  misc_feature (440)..(440): n is a, c, g, or t
gaacacataatcacaaatattctagcaaatgctgccttggttgcagcctgcactgtagacccaagggttttgctgtggctcttcttatctcccttggctcataaagccccagatgatgccagagcttcaattagagccatcatcatcccaggcagggatatctttgagaaatgactcagttcagccccaggcccctgtgactctgcttaaagcacacatttctgctgactcttgtacctggggcagcaggataatcaccaacacactcttaatgagaaacaacacaccaagcacagtggagctgtcctaggcaacactcgcggtctcaggctgcggtgggcgtctgtcctgcatgtggcccagaccaccctgacccccgggcctgcctgcctggccctgcatgctgcacgctcactgtatttgtgcagatcctggccagnacaaagtcgttgctcttg

SEQ ID NO: 151 - DNA, 386 bp, Homo sapiens
  misc_feature (360)..(360): n is a, c, g, or t
gtcctgtctcgccgtattgctgttgaatatagtgattcggaagatgattcagaatttgatgaagtagattggttggagtaagaaaaatgcattgataaatattacaaaactgaatgcaaatgtcctttgtggtgcttgttccttgaaaatgtttggtcattctagtgttttgctttcttttccttataataaatgacccttttcctccataacttttgatttctaaggaaaatattagcatacatttcaaactaaatgttttacagtggcttatcttttttttccccctgaaaaagactaatttggtcaaataacccactaagtattaagcatggacagctgttgttagagtagcagatncagttttttgatatatcttaattggg

SEQ ID NO: 152 - DNA, 476 bp, Homo sapiens
  misc_feature (457)..(457): n is a, c, g, or t
gctgactgttcttcagatgaacttttttttgttcaggttctaaagtcaaggctagggggcaatattagaagtcttttttggtcaacattaagccatagtttctaaatcagctttaggaggaataatgtatttggatattgatctgaccttttctttttatttctaagttggtttggcactgaaaaaggagaggtttgttatcagtccacacccctaatgccctgacatccatgaatccgttttagtggaatccagatctctaagaaagagatataaaatatttgttcctatccaactgtgattactgtatttccaactatgttgctgtcggtaagtagagtctgcatacatgtttctggttcatatatgcctagggttccagagcattacatttccatcaaaatgtagatcccgagaatgccagagagggtcaggccgaggatagaacagacagggnattttcagatactgcagta

SEQ ID NO: 153 - DNA, 358 bp, Homo sapiens
  misc_feature (357)..(357): n is a, c, g, or t
gagtagcatcaccagccttgggggtccagagcctaatttatattcactatccttccaagtcccgggtagcagaagggttgccatagatctcagtttgatcaaaaagaaggcttagaattctgcagttaagctgaggtttaaactaaaaaatgtttccttgggtcagtggttttgaggtccagtagctaggcttttctcttttgtccttcctgttggaatgaaaacatttcgattttccttcatctgtgactggtgccatagacacaggtttatagttttaacttacagtattgtttgaaatttacctgtttttcttgtcaaacctgagcactcctcctgctgaagtttcttatttant

SEQ ID NO: 154 - DNA, 438 bp, Homo sapiens
tgggctaaggcaggctcgggagagcccagcagctgaaggctaggcgccactgggcctggaattcgccacaggacggatcttacagaggcaagtggtcctggaattcgccacaggacggatcttacagaggcaagtggtccctggacctctcttgcatccattctctagacggccgtgtcagaggctccaccctgttgtgaacttggtatggaggcaaaggcttagaggctggaccagcattcttgggcaaggactgactctccaagggttttgttcttggctttggacacctgagaaccccctcctcccctcccccaatacaaggtttttgacatgagtgtactcctgcttagttcctcttgtggggctgcatttgcggtgctttgccctccccactgtgagtgaggggcaagggatctcctcaatcctgtctcccag

SEQ ID NO: 155 - DNA, 523 bp, Homo sapiens
  misc_feature (518)..(518): n is a, c, g, or t
cgaatgcccggcggccgaggcggctagagcgtcgcctcctcccggggaaccgcgtgtgaccttccagcccgcggaccgatgctgccggcggccgctcgccccctgtgggggccttgccttgggcttcgggccgctgcgttccgccttgccaggcgacaggtgccatgtgtctgtgccgtgcgacatatgaggagcagcggccatcagaggtgtgaggccctcgctggtgcacccctggataacgcccccaaggagtacccccccaagatacagcagctggtccaggacatcgccagcctcactctcttggaaatctcagacctcaacgagctcctgaagaaaacgttgaagatccaggatgtcgggcttgtgccgatgggtggtgtgatgtctggggctgtccctgctgcagcagcccaggaggcggtggagaagatatccccatagcgaaaaacggaacatttcacgtccgctgacgagggaagccgtgacaagtgagctatcaggaatcagactantccag

SEQ ID NO: 156 - DNA, 439 bp, Homo sapiens
  misc_feature (5)..(5): n is a, c, g, or t
atagngattgagatgtctattttattattgatagactattactaatgtcaatattgaacactaccctggaattcctgcctggttttcctacccaaattgtaccactccttgaagaactacaggcacagtaaaaaaaatatggcgtattatgtgaactaaaagagttctaaaggagttcttaaaggagtggtagaatttgggtaggaaagtgattaagtccaacttaaaaccaacagtctcaaacgtctacaactacaatgtccaatgagccactagccacatgaggctatttaagtaaatttagtttaaaatccagttttcgaattacattagccacattgtcaagtgttcaaatcacaggggggttagtggctactgtactgggccaacatacattatagaaattttcattataggaagttttatngggcagggctgc

SEQ ID NO: 157 - DNA, 481 bp, Homo sapiens
tcatattgaagaatttgttatcaggaaactaccagtcctgctttacaggaagtctgttatcagatatcagatggcaagttccccatgtcttcagatgttcaaacaatattgtggatggtctagaaagagtttaagacatgctgttaaatgtagggctagataattctctgattctttgatgtagtctggaaagaaacaatccattgtccagttaataaatatttagtgttttcatttttaagacactcacaatccacaaatgtccctagcaatttattatttttaaagaaaatgactttttattccttgctagtgaaaaatgtacaatttatatgctgcactgagaaaaataacagatatactttcttccattcattttcatcccaaacatataaaaaataatccattgattgttccttgcattgcatatcttattaaagatatttcctacatgcaactaaaaaacatgctgactggtgtc

SEQ ID NO: 158 - DNA, 450 bp, Homo sapiens
  misc_feature (145)..(145): n is a, c, g, or t
gtagacctagtacgctggaagattttaaaagataaacatatccctcaatttttacaacgaagctttggaaaaaaagattgagaagcagctggagtcacctatttttggcgttttaagcttttcctcctgaggattttatatcagncaatgcagaaaaccccttcagaagccttgtggaaacccacccatgaggactcaaaactcttactagtggcattcgcctgggatgggcaatgctgacgatgaacagcaggaagaaggcacttcttccaaacaggtggtgaagcaaggcctgcaggagaggagcaaggaaggagatgtagaggagcccactgatgactcgctccccacgactggagatgctggaggccgtgagccaatggaagagaaactcctggaaatccaagggaaaatcgaagctgtggagatgnacttgaccagggaacacat

SEQ ID NO: 159 - DNA, 586 bp, Homo sapiens
  misc_feature (557)..(557): n is a, c, g, or t
gaacaaatagaaaaagaggaaactttaaatccacgcatacacgtacacacacacatgcacacacacacacaccaaattgaacaaactggaaactctcactctgtgaaaagttgtattctacacatttctgcacagaccacaagcagtgttatttccctgatgtttgagttgttttgttcttttgtgtgttttgtttatttgtgtgttgtttgttggttttcgtttcttgagtttttgttttgttttcaccacatctgttatttccactagtttttttttggctgtgtcatgacaggcctcatgatgctaacagagattcttcccttcttttttccaaaagcaccaaaaaaaagggctgaagtagtctctgtagcatctccccaaagtgttaaacagctacataaggtgaagccaaggaagggtttcactagctttcattttataattatttaaagtcttatgtgtggatacccaagagacaaaaagttatcttccaaaataaataatcacagaagtacttttttaatgaagagcctatctgtttgcattctaaaaantaatttatggtttccattgtttgcatgna

SEQ ID NO: 160 - DNA, 622 bp, Homo sapiens
  misc_feature (575)..(575): n is a, c, g, or t
gcggtgagatattgtgaacgcaacaactggagctgtagttggcgtgtgtgagtgtgtacctgcggttctgtttagtggcgagacaaggtatgtttcagtactgctttcacggcaactgtgtaatgcatatgctctttctacactagcggtttgctcagttttgtctgtccggttaagttggagaaagaaccaagttgtgttgttctctggagggggtgctgctgcctctccttgagtagttctgtgacccccaatccagctgttttgtagagaatccttctagcttctggtatgcctgctctccagcagctggaaacagatcttcctgcctgaccaaagctccttgtttcttgcgtggcatgtttgttcttcccgatccggtggctgtcatagccgagggcctgcctgccaccttcctttatcatttgccctgttggggttcagggtgcatccttagcccaggacctgctcttccagcatcatgggttttttcctaaagatctcaacggctcccttgggctccataaatattccttttttaaaaagcagactcctttcatgcattggatgaacgncctcttgtctggggctgggctacagcaacaatgtgttgatccngtat

SEQ ID NO: 161 - DNA, 609 bp, Homo sapiens
  misc_feature (519)..(519): n is a, c, g, or t
gaaaatgttttgtgggccatttcagtgctcctcactccctcatagccaccagcctcatcagaaagatgccccgagagcacaggaagccccccaagcatgtgccccacccaccttctctaacaaggtaataaatatttccacagccagtcccaagctgctcccaagcctgtaggtcaccaggttggaagggcaggccacttctacctgttacatgccctgacacctcttatggatcaatcaggacattttttctcaatgacatgacctcagatgctgttactcagccacaaattgacccatgaggtgaaacctatttttatccatgtattaagtagtttttaataatccatactacacttgcttacagaaaggctaaagacacaaacaaaactatcaaacaccagcatcaaagagcaagaatggagtaaaggggaaatctgtgtgcagaagcccacagaagctgagggggcctgtagcaaatgagcaaaacttcactctcagcttccttacaggcaatgntaaaaggaaaataggttacaaactcgaacaactcagtcatttttagggaaggcatgaatctagnagcaatgctagcatggatttaatang

SEQ ID NO: 162 - DNA, 582 bp, Homo sapiens
  misc_feature (518)..(518): n is a, c, g, or t
gcctcgtgccacccaataatatatgtgctattcaaaaaatgtgatcatttaggacattatcacaagttgctatggaaataactgaagacttcctcaggagaaggaaagaaaatgaggaacataatgatagagtcggggctcgatgcagctgaaatactgaatactgaaatactagaaaccaacaaaactggtttctagaattctccagggatttgggcttgggaaacttggtaagaatagtttttatttttgaacattcaactttggttcttcaaagtaatctttaaacatgggaatatgttttcaagacaatttataggacatacttatgtagttggtgaggtcttctcaaaagtatctacctttgttagataataacaagtgattaagaaaggggagatatggttttatactcataacaatttcctgtgccattctctgtacacagaggtgattcatttactctcccattagtagacttgcactttgggctgtctgttctagtatttgggaagtgnttatgcttcttncagggacatcttatgatcagaggcaggatgtctgtgagatggtagctagagt

SEQ ID NO: 163 - DNA, 415 bp, Homo sapiens
  misc_feature (313)..(313): n is a, c, g, or t
gcaccagccagcctgactgtccccagcagcttccggaacaaaaacagcgacaagcagaggagtgcggacagcttggtggaggcagtcctgatatccgaaggcttgggacgctatgcaagggacccaaaatttgtgtcagcaacaaaacacgaaatcgctgatgcctgtgacctcaccatcgacgagatggagagtgcagccagcaccctgcttaatgggaacgtgcgtccccgagccaacggggatgtgggccccctctcacaccggcaggactatgagctacaggactttggtcctggctacagcgacgaanagccagaccctgggagggatgaggaggacctggcggatgaaatgatatgcatcaccaccttgtagcccccagcgaggggcagactggctctggcctcaggtg

SEQ ID NO: 164 - DNA, 478 bp, Homo sapiens
ggtaattgcaagaagttcttgagctgaattcctgttgtgacaacttcctataattacagtagataactttttcttttagtcgtatataacttttctataacttgtgatggacaagagatatgcttatccaataaaataagcttaaatattagatgctcttgggtcaaaatgtccttttaccaaattgacctttttatgagttctttgggtaaatactttaaagctttttatattttaaagaatacttgtaaaagcatatcacatcttaaaccagtggtgcacatgtggatttacagctcatggactctactgttcagctttaatttataaaacatatcacacatttaatgttatacagtatttacatatagtggaacatagggataactcagttttatgtaaatttttgttaagtgttgtagcctgcccagagtgacttctatttttcttctttgcctccgggggggaagcagtattt

SEQ ID NO: 165 - DNA, 422 bp, Homo sapiens
ggcaaagacctgagccgaggagaggatgaattcaagtttccttacctgcgtatctactaacaaacatgagacctccctgttacaggtggtcagttggccaaatgtactaacgggcacatgaaagaaagaacagcaaattaccaagtgtctcagaaaatgacaaaaccatattttgacaagtttatttaatccagtgtggtagaaaaggcacaattccaatgtatcatttagaattgaatgtcattaacctggctttgttctttggaagaaacaacttctttaaagagcttctttggctctagaaaaatttcaaacaattaaaataagaaaaaattttaaactggttttttataatgaaaatatacatacctttgtgtatagacaagcccatgcccattaaaataagttatcctgtaacataa

SEQ ID NO: 166 - DNA, 477 bp, Homo sapiens
  misc_feature (402)..(402): n is a, c, g, or t
ggcagcctgcaacttcctggagaacaactcagtgtcacattaaagtttattatgtatttaatgatacactgtttaattgacagttttgcatagtttgtctaactttagagaattaagagcctctcaactgagcagtaaaggtaaggagagctcaatctgcacagagccagtttttagtgtttgatggaaataagatcatcatgcccacttgagacttcagattattctttagcttagtggttgtatgagttacatcttattaaagtcgaaattaatgtagttttctgccttgataacatttcatatgtggtattagttttaaagggtcattaggaaaatgcacatattccatgaattttaagacccatagaaaagtttaagaatgcttaattttcttatccngtaatgtaacacagagacgaacattgagatgtgcctagttctgtattacagtttggtctggctgtttgagttcta

SEQ ID NO: 167 - DNA, 402 bp, Homo sapiens
ggtgatctgctcgggacagctctgttagccttaagttttcattttctttggcttattggagatcgagatggagatgttggagactaataaattctacaaactgctctcaagttaccaaggaagaaaatacacgacaaccacttatggctctttttcaaaactcttaaatcagtagtttgacttttgccagggtaatcttcagttggccctgattcaattaaatggccttaatttttttttaaggaatttgtgtcaaaaccagaatgaagagtattcgtgctgcttttcatagaataaatgataatttgacatagacatagatataagttccagctctgaaattaattaggaaagaatataggatgtttacagtgaataattttaaaccacaacatagcagaa

SEQ ID NO: 168 - DNA, 454 bp, Homo sapiens
gcttttacttataacagctttcaatataacctgaagagcagggaagtaaaaaaaaaatttaaaaataaattcaataagatgccattccaataacagggagatagctttttgattaccaccttaatctatgaataaattttagaacctatccttcttaggcaaaactccacaaatattatttctcccaattagtcgttgaataggaattcaagtgcagatacttcaaagttatacttaccaatgattatctggagagcaattactctatgtggccacaagtataaagccatattctccaaagattagcacatctttgcctaatgttaacatcctttcaatgaatactaaggccttgacaaggtgcacactgaaagagaaggacaccacactctgttcagtttgagctgatgggaataacagcatcacaggacatgggcctgcttttccaaaagac

SEQ ID NO: 169 - DNA, 304 bp, Homo sapiens
ggttactgagaacaaaaacccagattggtgagataggacacttgtgcagcagatatgccaatgggccatgtttattgtggattggtaagaatcaccaggaaaccattaagccccaatagctacaaggagggtggttaatctgctatatcaaactccttccctgaaaccagcaaacaccgggaaacattttggctcattataatccggtgaacaatgcagtcaggcctgttataaccgctgagcagccacactcgcacctcctgggtgctgtagtctgtgttggtacaggcttctgcatgcctgg

SEQ ID NO: 170 - DNA, 408 bp, Homo sapiens
  misc_feature (283)..(283): n is a, c, g, or t
ggatccccaattgactcagagtttgaggagttagtatcacagaattagatttttttaaagcatttgtacgtttccattcccaaatatgtagctgtggttcttgaaaacacatcctacattgcatatgggcatagcagtttttgacccaggcagaataagttaatatttaattaaatattgctttgaagatggcgctctgggcatgagcatggggctccatgacttcccttctatccccatgagcccctcctccatccagcgacaagccatgggcatgcatacnatgcagcaagaccaacacaagagcaatattgaattgttcattctatctaaaattacatgtatataaaatatataatttatctncctgcatttttgaagnataaagtcataaattgtacatatctg

SEQ ID NO: 171 - DNA, 569 bp, Homo sapiens
  misc_feature (548)..(548): n is a, c, g, or t
gcaggtcttactggggaaaaggatgggagtgggggctccccaggactcgattttagctaatgcgctgtgtcactgccccagctcggacgtagaagcccagccctccgtgagctcttgggaaaggggtgaattcactgggtcatggaagggacagtcaggtgaccagcggggtcgccagatgaagcttcccagccgggaaacaagacggggtttcttggcaggccctggtcctggggagcaggccctgttgttggctggagaggaaggtgtggggtggaacaggtgtccacatagctccatctctgggggctggagcacacactttgatgagcccccccggaaatgatgtcagagcctagccgcttccttatttgctcttttattgaggccgggcaggccctgggtcactttggaggcccctcttggtccacactggactggccgggaggtgatgggcggggaaggttctcgtgattgattgattctgagtctgagagtggcgagtggggagaggcttcccagttctctcagcttccctgcagctgcanctgccctctggtccagtggtg

SEQ ID NO: 172 - DNA, 490 bp, Homo sapiens
  misc_feature (421)..(421): n is a, c, g, or t
gagattccacatgaactttaaaatcagcctgtgaatttctacaaagaagccagctgctttcctgatagagattgcatagactctgtagatcaattttgggggtattgccatctgaaaaatgttaagtctcctgatacatgaacatcagtctgcttttgggctgccataacaaaataccacggatttggtggcccaacaacaggcatttattttctcacaattcagaagtccaagatcgggtgcaagcaactttagtttctggtgagggctctctcctttggttgcagatggccgctttcccactctttgctcacatggcctttcctttgtgtgtggagagagaaagagttggagggaggaagagagagagagagagagagagagagagagagagagaaagagagagtgagacagaggcaantgctccctggtgtctcttcatatgaagatgcaaatcctgtggaagcccttatgaccttacgtaacctta

SEQ ID NO: 173 - DNA, 434 bp, Homo sapiens
  misc_feature (24)..(24): n is a, c, g, or t
gtggcggtacatttcccaggaacnctatgaccatgattacgccagcttggcacgagggcaccaaataaatccatctagctggtagttttatctagccaaataatttaacctagctcccagttaattgttggcataaagagagagattgcctgttagttatagagaaacagtgagtcaagtgcttaatttgaaagtaacaagctcagaggaagccactccttgccctcgttaaagagggtcctgttcctctcttcctgttgatgaaggagcgtgagggtgttagcagtgtgagatctttggacacccatggaatcctttcctccacaccacccgtgcacttgccaaagacagggatgaggcaagaggttcttcctgaggcccggccgatcctcaggatgcagagaagtcatgtanacccaccagccccaccttgg

SEQ ID NO: 174 - DNA, 433 bp, Homo sapiens
  misc_feature (425)..(425): n is a, c, g, or t
agggccttgggcacatgcagactggtgttgggatgtcactggcaataccagtagaagttaagccctgtctgagtgtttcttacaaccggagttatcagataaatgaacattacccttgtattactccatgttttgaaaggtgattatttgtgaagctgataaccgacccagactgctttgtgacatgaagctatgggtaagcgttttgtaaacttctgttcaaaaggaaagggatctaaatgacatcagaactcttcaggtaccagcaccaggaagactgaatatcctttttaatgcaccatgaatcctgggagactaagcaaattaacagtatgtcaaattctgttacaacaaatccctgtatgacaaaaatgttcaagtcctggctgatggtccaaatatttcaaaaatattcagtacaacanaaaatttg

SEQ ID NO: 175 - DNA, 543 bp, Homo sapiens
  misc_feature (355)..(355): n is a, c, g, or t
ggtataaattaagacaccctattgacttccgggtaaggggagtcaattgattacccagcagcacagtatttgctttttataattccctttttaaatacttgttcttaattcactggttttccttttctgtcatttttcagagtttagattgtgagtccatgttttgtctgttgtgcctataaaggaaatttgaaatctgtatcattctactataaagacacatgcacacgtatgtttattgcagcactgtttacaatagcaaagacttggaaccaaccaaaatacccacaaatgatagaccggataaagaaaacgtgacacatatacaccatggaatactatgcagccatagaanaggatgagttcatattcttcacagggacatggatgaagctggaaaccatcatcctcagcaaactaacacaggaacagaaaaccaacaccgcatgttctcactcataagtgggaatgaacaatgagatacatggaccagggaggggacacccacctggggcctgttgggggatgggggctaggggggga

SEQ ID NO: 176 - DNA, 482 bp, Homo sapiens
gctgtttctgactgaggttccccccagcttgaatcttgtagggaggcaaggagcagagaacaggccctgccctcacgttggtccacatggccccctgccactggctggccacatgaccttgggcaagtctcctgtaccttccaggccccagattcctcatctgtaagatggggagtaatgactcccccgtgctggggttgcacgtgttggcggagatggacaaggagccccttctagcaggcaagctctttccactctccctgcccctcgtagtggcccctagatgtgctggctggtccccatcatgcccagttcgccactcacagagccgaggccagagaggtcaccaagttcgtaccgtctgtgctacagcccatccccaagattgcatctccccagaaggtgcagtggaagaggagatagtggggggttgaggctgtcacagaaggtcagagccagagggtcctccaggacatggaggc

SEQ ID NO: 177 - DNA, 552 bp, Homo sapiens
  misc_feature (523)..(523): n is a, c, g, or t
ggtggtgattgagttttatgcctctacaaaataggttgtgaattaacttcaacatctgttgaaatgcatcaactgggaaatgtccacctccttaatacagggctcaaataagaataataccttgggataccacaacatttacatttgataccccctgtgatcttagaggaagctactatacctaaaacaattatgccttcctttaaccatgaacatagtatcatttttgcctgcattctctataaatgtacccattgtgcaaagatatcccacagtggggatcgattccttgacttaatgagtcatgtattaaaattaaatattttccattttcaaatgttcttgacaaatcgggaacccctggacccactagtttaaataagttaatatggctaaacataatgttttgcaatatgtaaatgttttgaaggttattgggtattccatttcatccttttgcgagttttagtagattgggtctttcaaggaattaattggtccaactgatctagttatcaaattngtgggtatggagtttataatcttctaatt

SEQ ID NO: 178 - DNA, 617 bp, Homo sapiens
  misc_feature (573)..(573): n is a, c, g, or t
gcatatatgctgtgagaagttgtaatattaattggcctctttgggtgggactctaagcagtatttgcagaaatatgcttctggtccaacttgtacatccagaacaaaagggcccctctagtagctgtgtgctggattattcaggactgattaactcaagttgctcattgaatcacatcatccactttatcccagaaactagaactgtgagaaaccaggtttagaagacagtcacagaacagcacacatccaaattcagcgccattgaaagttggcctaaggctcagtgccactcacccctccctccccaaaagacaagagaaatttggccagatggggaaggtcactggaaattgaggccaaagggctgatcagaattgcctttataatatatttgatggatgcctataaatgtgggttttgcaaatattgtttaaaaaccaaacttgaagccaggcatggtggcccacacctgtggtcccagctactcaggaggctgaggtgggaggatcatttgagcccaggaggtcaaggctgcagtgagctgtgatgacccactgcactccacctaggnacagagagacacctctgtctctgaaaaacaaactgaagtttgaa

SEQ ID NO: 179 - DNA, 397 bp, Homo sapiens
  misc_feature (394)..(394): n is a, c, g, or t
gctggaagtttcagcaataggtttaacaaccttttaaatgttaattttcagttaatcaacattttagataatgacaaattaattaagtgggtttttttggtggtgggatggatggtgctttagttttacttttgaatacaatttcatttatatatgcacagaagtatacaaatcataatgtactgctcaattgattttcgtaaaagaagtgctcccaagaaatcagcaccaggtcaataaataaaacaatgacagaatctcagaaacttctttccagttcctacctcgcttaccctaatggtaatcactaacctgacttccaacaccagagattagctttgcttacttctgaacttcatataaatggactcataccaaatattctcttttgtgnctg

SEQ ID NO: 180 - DNA, 388 bp, Homo sapiens
ggatttatttatgttggtacatccaacaaaatagttggagccacaaggcccttgtctttttaaaaaaacaaaatattttgaagatggtgaaagtgactgattttaataatgagataggtcacttaaaataacagttcctttgaccttaaaacatggcactggcccatgtgacctttgccatttcccagtacttccctttacagtctgcagcctataaacagtggtgtctcaaagcaaaggaaacctccacaagtgctgcaacagtgcattagtttttatgtagcaatttgtttataaaaattcccatcccataatgcatgaccttcctaaaatacccacagcctcataggcaggtgagctttctgtgctaccactggttagattcagc

SEQ ID NO: 181 - DNA, 277 bp, Homo sapiens
gctgcgagtatacagtatacagaagaagcgtgtggctttcaatgtttcgaagatgctgtctggaataggggctgaaggagaggcaagaagctaccggatgtttcacgacgacagcatgaagtctttcttccgtagactgagtttcactcccgacggatctttgcttctcacgccaggtgtggagctgatgagtctgccctaccgcctggtgtttgctgtggcctcggaggattccgtgcttctgtatgacacccagcagtccttcccttttggttac

SEQ ID NO: 182 - DNA, 193 bp, Homo sapiens
gatgagaatcagatcgagtccaaggcagcctatgtcctcttctaccaacgccaggacgtggcgcgacgcctgctgtccccggccggctcatctggcgccccagcctcccctgcctgcagctccccacccagctctgagttcatggatgttaattgagagccctgggtcctgccacagaaaaaaaaaaaaaaaa

SEQ ID NO: 183 - DNA, 279 bp, Homo sapiens; identical to SEQ ID NO: 28
  misc_feature (17)..(17): n is a, c, g, or t

SEQ ID NO: 184 - DNA, 531 bp, Homo sapiens; identical to SEQ ID NO: 29

SEQ ID NO: 185 - DNA, 534 bp, Homo sapiens
gccacattaaagtgggaggacaggaattttcaaccttggacaagcagattttctgtctgtagaaaatttggaatcaaatctgacacccgctttaggtccagggcctcctggcccaactactgagctccttccacagcccaggcagctaggcaagcccgtgccagggccccctccactggtgtggtgagctctggtccctagccctgggagttgtggccccgggccaagatccacccaggccctcacaagctggctgaagtgccgtggcgcagataggcctcctacccccagtttcttcctttgtcatagcgattcacagccactgcctccaaacctctaggcccctccctgctggcatttggatgtgtttgctatgtacctttgggccagctgtgtcagaactcagccatgggtaggctctgaggggcacagtgccccctcaccttaggcaggaagcacccccagactctggtcgcccctctcccattcttagctgctggagccagcactgccagatgagtgtgctggctcaga

SEQ ID NO: 186 - DNA, 494 bp, Homo sapiens
gagcattctctctgacatttaaaaataatttctattcaaaatacatgcataattgattttacacctcattactggtggataatttatgtgatgtggattgctggtgtccagcatgacccataaacaggtcagaagaatgatggaatgttttagaataaactcctgcttatagtatactacacagttcaaaagatgtttaaaatgcttttgtatttactgccatgtaattgaaatatatagattattgtaacctttcaacctgaaaatcaagcagtatgagagtttagttatttgtatgtgtcactagtgtctaatgaagcttttaaaatctacaatttcttctttaaaaatatttattaatgtgaatggaatataacaattcagcttaattccccaaccttattctgtgtgtagacattgtattccacaatttgaatggctgtgtttactctaataatgaatcagagaagtgaaaaaaaaaaaaaaaaaaaaaa

SEQ ID NO: 187 - DNA, 375 bp, Homo sapiens
gcttaacatggatattggagtgtctggttttggacctgagactgctattgacaggtcaatggcaatgagtgaagactttgtcatggaaatggggaagcagcccataatatttgaaaacccaatgtactcagccagagacagtgctgtcaaagtggttcagccaatccaggtgactgtatctgaaaatgtggataataagaattatggaagtcccataaacccttctgagatagttccagagacaaacccaacttcaccagctgctgatggaactcaggtgacaaaatggaatctcttcaaacgaaaatctaaacaaactaccaactttgaaaatccatctatgcacagatggagaacgagcaaaaggaagtgttg

SEQ ID NO: 188 - DNA, 546 bp, Homo sapiens
ggataaatgcatgagttgtgctccacatttctcaattgctgactgaaactgcatgattaaacataaatacagccagagctgcagaggagcatggaacctggagaaacagatgagataagcttctttagtctctatctgtgaaatgcttactttattctagtctcaattgaataaagtgttaagtgttagttctaacaactgaataagttaaatggcccctacttagagcctggaaattacaaataatcagatattcaatttcctaagttttcagagatttacgggttacacagtacagcaattcatttgattacccagatatcattcttaaatatctagtactaagcttttcttaaatttccttcaatttgttgtttctttagaagactttaggtgttaaaatttagctttgtttcttcatttaaaagttgagtataggcattttttccaatgaatacctttattcttattacaggccatccgagttactcatgagacagtgccatgaaggcagactgagcaccagtatagatgagaagtagactc

SEQ ID NO: 189 - DNA, 319 bp, Homo sapiens
gtttttggacaatttaaccttaccctggcttgggaatcccagtgacattgatgttaagggtcttcttcctgtgtaaccttaggaaagtcattcagcttccccaagccttcgttattatgaaatatataaacaaaactcatacctacagtttaaagaagaaataatgaacacccatgaatttacagcgacccagcttgagtaatggagcttcagtttcttatctgtgtaatggaattttagactggagactctgagtttatgtcaaagagcaacctctgtaactgcaggaattaaaggacaagggatagccaaaaaaaaa

SEQ ID NO: 190 - DNA, 316 bp, Homo sapiens
  misc_feature (246)..(246): n is a, c, g, or t
gcttagcttctgtattcctactgccgcccagaaaaggggcagggctctgcagccgccaggacagacgagcaccccatgcctatacctccctccccgagctaagtcccagggcatctgggccttgcctggagactgggctagctctgtaggctcggagagcctggggagggtgccaaccccacctctagtattttgggagatagggaaagtgaaccgacttccccttcccatacccctcagggtggntnccctaccagccaggcttactacttctagaagaaagcagagtgccagggagtgagattgcatccctggn

SEQ ID NO: 191 - DNA, 368 bp, Homo sapiens
gggaacctccacttgttttggctgctgcaaatgtggtgagaaatatcagctataaatatcgagaggacttgtctgcacatctcatggtagctggctgggaccaacgtgaaggaggtcaggtatatggaaccctgggaggaatgctgactcgacagccttttgccattggtggctccggcagcacctttatctatggttatgtggatgcagcatataagccaggcatgtctcccgaggagtgcaggcgcttcaccacagacgctattgctctggccatgagccgggatggctcaagcgggggtgtcatctacctggtcactattacagctgccggtgtggaccatcgagtcatcttgggcaatgaactg

SEQ ID NO: 192 - DNA, 329 bp, Homo sapiens
gagcttttcccacacagagtcagacatctagagccttagcaatatgtatttaaagatgtttcacacagaattcagaacacctgttccagagtggcttgggttgttgggaaaacaataccaaagggtatgcctttgggtaggtgggaagtggatggacctgaatggattcccaccttctactactactacttaaaacaatatgtgactgttgcattcaaatttctcagagttttaagtaggcaactcccacttagttccaggtgtatccaagggggcttttcatatctgaagctctaaacctcaacatggaccccctcagactggctgtt

SEQ ID NO: 193 - DNA, 398 bp, Homo sapiens
gaggagtcgggggcgagccttagcagctgcaccgccatccccatgctggttggtgctgccctgcctctcgtgccgggtgttgcttcagcccagagccagagggctgggtcccgggtcctccacaggtgaccccggtggacacacgcgttcccatcctggcctccgtctctgcttttccacttctacctgcgtgtgggtttgccgccttgtcatcggttgtgtgagtgtcgcagacctttccagagctccggttcactctttccaaacaggcctccctgtcggtggcactgcactcctagaaccttcagtttctacgatggtttgtttggtccttttgaaccaccccaagaactcaacatggcaaagcaaatggtaaaagcttcccgactgttctactt

SEQ ID NO: 194 - DNA, 322 bp, Homo sapiens
ggaagtcggcggtgtttccattcggtgatcagcactgaacacagaggactcaccatggagtttgggctgagctgggttttcctcgttgctcttttaagaggtgtccagtgtcaggtgcagctggaggagtctgggggaggcgtggtccagcctgggaggtccctgagactctcctgtgcagcctctggattcaccttcagtgggtatggcaggcactgggtccgccaggcgccaggcaaggggctggagtgggtgtcatttatatcatatgttggaaataataaatactacccagactccgtgaggggccgattcccttctc

SEQ ID NO: 195 - DNA, 272 bp, Homo sapiens
gcaccgcccgcccagcgttccaggcacatgaagagaaagcattccaaagcctctgattgttgtttcctttttctcctcccgaaggaacagctgattcatgctcctcccgcaattgtcacgtctgtgatttatttggtgtttcgggcgtggcctctggagccccggcacgtggtgggccacgctgctggcgctcatgggccctggtgtttgcaccgcactttgtaatcagtcccgtggttgtctgtacagaattaaactattttccgatgaaa

SEQ ID NO: 196 - DNA, 370 bp, Homo sapiens
  misc_feature (342)..(342): n is a, c, g, or t
tcttaagccatttttaggaaacaggaggtttagtcacgtgctcaacaaaagcacaacaaatggggagcatttaatggtgtaagggctgtgaggtgtagctgctgaaactgtagctaggagctgccttgctgccttcttgcaggcagattggccagatgagccaggctaaaatacaattaatatctaccattgtggtttaatatgaaatatggatacctggtctttgtctcagttcttgtcatagagttccccaaacctttagaacttcctgagtggtaggaatatctcattagtcataatgagcccctttgatccataactcctgagtttatgctaagaggntacttaaggggggccctaaaattcttaa

SEQ ID NO: 197 - DNA, 284 bp, Homo sapiens
  misc_feature (280)..(280): n is a, c, g, or t
ggaagtggggtcaatgggtggcttgaaagggattttagagctctgtttttatttttcctactttacatcagtgcattttgggcaaaataaaatgagtaatgtttacttttaggcctcagtaatataaaaaaaggcagattctaaggattgtggaaggtcatgtttttgaaagtgaaaggtaattcagctccggttattaggagaaactctgtctccatcttaactcatatttcagttgagcagtaacatttggttttgtgtcctctcttttttttttttnactt

SEQ ID NO: 198 - DNA, 227 bp, Homo sapiens
  misc_feature (200)..(200): n is a, c, g, or t
tggggtttctccatgttggtcaggctggtctcaatctcctaacctcaggtggtctgcctgctttggcctcccaaggtgttggggttgcaggcatgagccactgtgcctggctgacaaagctttttaaggaagttctcaacccttattacacataattcatactggacagaaaccctacaagtgtgaagaatgtggcaagnctgtaacaagttctcaattcttttatt

SEQ ID NO: 199 - DNA, 578 bp, Homo sapiens
  misc_feature (382)..(382): n is a, c, g, or t
tggtgagattcgttggtggctgcgcgattgtggcggagttgcaagcaaacgggtttcatcaccttaaatggttttgaaccaaagaagctgtattcccttaaaaagacggacaacccatcgtgtgaactatagagtttgtgaacaaatttatattgggttcatagtggcgtcatgcacgcagactcctgcgagttcccctaagttcttagaggactgctttgccttttgatctgagagttgcaaagttccctaaagaatggcccttgtggataagcgctaagtcaagagacagcgattggacagaatttgtgaggaattcgtccccagatcatgaaagtcaccctgaaccccgcctcgtggtggctctgctggatggacgggnactgcactgtggacatgcccatcttgaaggacctggccacctggccttctgtgaggctcagtccatgcaggagatccacgaaaagttctaaacgaancgtgggcgccatgatgtacacacntctcntcacagggagactggaaattcaggcctganatgacgncagtggcaggctcncacggnctcagntgtgcgg

SEQ ID NO: 200 - DNA, 506 bp, Homo sapiens
gagagagctagcaccattctgagtgaatcccaacatttccccgtttttgttttcggtatcatgtctttttgatttcagattgcagatatgtacagccataggattgtcagatggtgtcacaagaaggatctgaagtggagcttctagtatccccaggagcgcgaagtgaacacggaaggtacctgcaggatccaattgtgtccattgatctctcagagtggctgaggataatagagtttcttcttcaaggtctcaaggtctgaagcatcccacagaatgatcctactgaataactcccataagctgctggccctatacaaatccttggccaggagcatccctgagtccctgaaggtgtatggctctgtgtatcacatcaatcacgggaaccccttcaacatggaggtgctggtggattcctggcctgaatatcagatggttattatccggcctcaaaagcaggagatgactgatgacatggatcatacacaaacgtatatcgtatg

SEQ ID NO: 201 - DNA, 528 bp, Homo sapiens
gttcataggactcgacaagagctatctggtgattttctcattagtaacatgcaacgttgtactgcaaaatttcaatcaacatgacaacttataatgagtggagatttcatattaggtactaaatattatagtattatttctattttctttttccaaataagaagcttggattattttattttgtggtctttatcattaactttaattctttctgtactgtgtataatatttttatattattggccttaccataaaattatttagaaaggttgtcaaaataagttatacctctttggcaatagatagatgtatacatctacctactatgatctacaattttaggttaagtgaagcttgggggggctactgacttggttaccttcttgtctcttgtcccaaagatttaaactgtgtacctttgtatagctcttctgccccattttgacttctgagatgaagtatttactaaattaaaaaaaaaaacaaaaacaacctttagctcctaacttatgggttctgagggaggaa

SEQ ID NO: 202 - DNA, 393 bp, Homo sapiens
aaaagtaagcacttttacacaagataaactttctgccctcaagaataaacaatcaagtgaaggaagaaatacaagtttgtggattatagcaatataattcaattttatagtgccaaataatgctatttctccctaatagtattctgcgataagagatccttggagatgttaagaggtgacaagaaaaagttaaataattttacacaatggaaagcatattctcttctcagagacattcacattgttatataaaaggctctggaaggtttgtcagaagaaacgtatattagagtttttctaacccaacattttacaagtaatttgagcagagaacgattctctcacaacaagatcaagatgtggtggctcattaaatcagaaataaatgtatta

SEQ ID NO: 203 - DNA, 487 bp, Homo sapiens
tcagttcagcaggaacaggagtcgagcccttgagcaaaaagccttcgtgtctgtaagtgcccgaggctcaggagagctggggctcccactcgcggcagacaggcccgcgtccaccctgcgtccaccccggcccggcggcagcacggtgccagtcatctgcatgtgctcctgcggcgtgggggtttgtagacttggaaaacccctttggcagaaagttaagagctcccaggcctgaaggcaggacacagtgccagcagggggccgtttgcccccatgtgtaagggaggcagggcccagctctcccacggcgaggtgagctgagactttctgagaatagttagagtgggtgcgtgtctgaaatgggtactcctagcagctattgtgtacgcaaagcctgagtacagcctgctggtgtcatggccacgtgtgagcaggccagcgtcacacggctcgctgtgacccgtcccgaaactgaaatgggcctggg

SEQ ID NO: 204 - DNA, 450 bp, Homo sapiens
  misc_feature (313)..(313): n is a, c, g, or t
cggggatggttgctctttgtccagagggctggacccttagctctgtcctcgctggagctgtccccgagcggtgatcccagggtcgtgacagagcctgtgaagtactcaagccgtcctccctgccactttcctacgggacttcccgggtctttaccatccctctacgtcctccgcatctgcttctcccgaagtgttcgggttgttgtcttcccacctggggggccctgggtagcccaagggccccggccccgagggagagaggtgagcctttctcaagggtcctccgtggacttttgcgcatgggagaggcggngcaaagagaatttgtctttgggggagttccttgacttgtctccttgcttccctccggttcttccttcccctacgatccccgcctcttcgcatctccgttggcgtctagtaactttctgaatggagagaagacatagg

SEQ ID NO: 205 - DNA, 526 bp, Homo sapiens
  misc_feature (512)..(512): n is a, c, g, or t
gcaacctgcaggatctgcagtgacggcagcctcggcctgggcaggcccaaggccacggtctaggacacaccttccctgagactcatgacatgagcctggggagacctcatttggtttccgttcagaagaccagcgtagcccttttgagacttttgggtacatttatcaaaaatgttttccttttgttttaccaggaatgagttcgggctatactctttgaaactgcctttgtgagcaaagtgctttggcaaggcaagggtctccctcagcagggctaggtgcactacagcctctttccccgtgtcccgttggatctgagctgtgtagcacttgttgcattacaaagaaaaagacttctgcaggaaatagggtagagaacaggctgtctagtcagatgcagcttgatctgtggcaaaaccaagtgtggtctttcagcttcagtgtgttagcatcccagcacctgggatggggtttgtgggcaagttgattttgccacactgtagggcccaggnggtcccattgccac

SEQ ID NO: 206 - DNA, 546 bp, Homo sapiens
  misc_feature (439)..(439): n is a, c, g, or t
ggtaagaaactcttgatagctatgtctattttatatcagtcactgagactttttttttaagtttttatttattattaagacaactttgccaaaaaagtcccctaagcacaactatttacatttctttatagcctcttctgatctctaacacatatgcagttttaactgttattttcatagtaactgatcttttgtctaaggatttttacctgaaagcacaatgtattgagtctcttgaaaatcatctttcagatctttttacagaatgaacttatgcactgctactgtagtattctcaaggaatatatgtaaacacaaatgtatgcctgaggttggtttttgcagaaaacagtctctgcttctaaaaacttctatgtctagtcttccataggaaatcctcactgtttaaccatgtgaggagcctaagtcattaaacggnatcatgtctgtacattgtgtatgaatgaaaagcacataaatgtaatctactttgaacttgtaaaaatgatggtgggngctattctggttctcatctcagtccgggtg

SEQ ID NO: 207 - DNA, 543 bp, Homo sapiens
  misc_feature (507)..(507): n is a, c, g, or t
gggggtccctggagggcaagccctgcccacagaaagccaggagcagccctggcccccatcagccctgctagacgcaccgcctgaaggcacagctaaccacttcgcacacacccatgtaaccactgcactttccaatgccacagacaactcacattgttcaactcccttctcggggtgggacagacgagacaacagcacacaggcagccagccgtggccagaggctcgaggggctcaggggctcaggcacccgtccccacacgagggccccgtgggtgggcctggccctgctttctacgccaatgttatgccagctccatgttctcccaaataccgttgatgtgaattattttaaaggcaaaactgtgctctttattttaaaaaacactgataatcacactgcggtaggtcattcttttgccacatccctatagaccactgggtttggcaaaactcaggcagaagtggagactttctagacatcattgtcagccttgctacttgaagnacaccccatagggtcggagggctgtccccctgcccc

SEQ ID NO: 208 - DNA, 418 bp, Homo sapiens
  misc_feature (225)..(225): n is a, c, g, or t
gttttgcaacaatctctgataagattggagatctgcagagaagctatgttgctgcgcagaagtctgaagcagcatttccagaccatgtggacacacaacatttaattaaacaattaaggcagcattttgctatgctctgattgttccttagaccacatatgttcttatgaagcagcattatgcaaggggaaaaaagcactatgtctgtgtatgtatgtatatagngtaatacgtatattttaacaaacctgtccttgatattagttaaggtgacacataagggtgacacagaatgtgtaatgcaaatttcatagtaatagtaactttataaaataatattataaaatacaggatttaaacctttctaaatagatcctgaaactgtctctcacattatatagtagatgtttgtttataa

SEQ ID NO: 209 - DNA, 463 bp, Homo sapiens
gacttatgggaaaactgggacacaggaagacaggtaaattacccaacctcacacgttaagtcagaactgggagccataattttgtatccctggtataaatagacaatctctcgaagaaatgaagagatgaccatagaaaaacatcgagatatctccagctctaaaatcctttgtttcaatgttgtttggcatatgttatctttggaatttagtgtctgagcctctgtctgttactgtagtatttaaaatgcatgtattataatcatataatcataactgctgttaattcttgattatatacctagggacaatgtgtaatgtaagattactaattggttctgcccaatctcctttcagattttattaggaaaaaaaataaacctcctgatcggagacaatgtattaatcagaagtgtaaactgccagttctatatagcatgaaatgaaaagacagctaatttgg

SEQ ID NO: 210 - DNA, 508 bp, Homo sapiens
  misc_feature (5)..(5): n is a, c, g, or t
gagcncctctaccacgaccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaacaaaaaaccgaggccctcaatccaccttcaggccccgcccccatgggcccctcaccgctggttggaaagagtgttggtgttggctggggtgtcaataaagctgtgcttggggtaaaaaaaaaaaaaaaaaaaaaaaaaatnggggg

SEQ ID NO: 211 - DNA, 559 bp, Homo sapiens
  misc_feature (199)..(199): n is a, c, g, or t
ggtgtttttcaagaatcagaaacccagaccaaccatcaatgtaacttgtacacggctcatcgagaaaaagaaaagacaacaagaggattacctgctttacaagcaaatgaagcaactgaaaaaccccttggaaatagtcagcatacctgataatcatggacatattgatccctctctgagactcatctgggatttggcnttccttggaagctcttacgtgatgtgggaaatgacaacacaggtgtcacactactacttggcacagctcactagtgtgaggcagtggaaaactaatgatgatacaattgattttgattatactgttctacttcatgaattatcaacacaggaaataattccctgtcgcattcacttggtctggtaccctggcaaacctcttaaagtgaagtaccactgtcaagagctacagacaccagaagaagcctccggaactgaagaaggatcagctgtagtaccaacagagcttagtatttctaaaagaaaaatgatcttttccgacttctaacagtgactatactagcataatcntctagtaaac

SEQ ID NO: 212 - DNA, 520 bp, Homo sapiens
  misc_feature (496)..(496): n is a, c, g, or t
gtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatcttttnacttctaacattctgtagtttagg

SEQ ID NO: 213 - DNA, 519 bp, Homo sapiens
  misc_feature (421)..(421): n is a, c, g, or t
gtagaaataccagcagtgcccatactccatagcatggtgcaaaaattcccaggcgtgtcatttgggatcagcactgattctgaggttctgacacactacaacatcactgggaacaccatctgcctctttcgcctggtagacaatgaacaactgaatttagaggacgaagacattgaaagcattgatgccaccaaattgagccgtttcattgagatcaacagcctccacatggtgacagagtacaaccctgtgactgtgattgggttattcaacagcgtaattcagattcatctcctcctgataatgaacaaggcctccccagagtatgaagagaacatgcacagataccagaaggcagccaagctcttccaggggaagattctctttattctggtggacagtggtatgaaagaaaatgggnaaggtgatatcatttttcaactaaaggagtctcaactgccagctttggcaatttaccagactctaaatgacgagngggatacactgcccccagcaaaa

SEQ ID NO: 214 - DNA, 577 bp, Homo sapiens
tattctctttatttctgcttgggatcaaatgttcagtttggatgatgagaatgaggcaagtactggagacaatggctggctctcctgtttgtcccaataaactccaatggaggaagaagctaaacaggttttggatggtttcggtctactgcatctagcttatcaaatctacagcctggtacttctggaacaggacttccctgccccagtgatgacagttaaagatggcaaaaatgaagaattcaacatgtatacgcagtgcagtctgatgtcttcagatgttctctaaaattctgtatttggtcagagctgggagtgagatcagggtgcttgcacatttctgctccactgctcccgattctgctttgctgccttggagggagagtagatttagatctgggggctggtgggggccatagccttggcagcttgctgggctaaccagtacttatatcttttggtcttgggcttctcaagttcacgacagacataggcactctcacagtatcaagcccatttaccgtcacctcacaccaatactcgccccaccgtgtgataggctctgctggtaacttta

SEQ ID NO: 215 - DNA, 349 bp, Homo sapiens
gaccacgcccagctaattttttctgtatttttagtagagatggggtttcaccatgttagccaggatggtctcgatctcctgacctcgtgatccacccacctcagcctcccaaagtgctgggattacaggcgtgagccaccatgcccagccctgcaaagagtatttttctagcgcctgggctggacagttgtttctaaaactaaactattttggtacctgcttctgggccagtccccagcctgtcatgagtgtgtgtgtgcgggcatggatgtgactgggagctctgctgggcacccacatctggggcctagtcaggtgtgtgtgtccggcggggggggggggggggggg

SEQ ID NO: 216 - DNA, 518 bp, Homo sapiens
  misc_feature (497)..(497): n is a, c, g, or t
gttttctaattgaaaaatctttaagtcattcagaagaataaaactgaaaatgtttccaataactgagccctagaagctcttaggtctgaagggttcagtcccagtgaagtccacaagggcaatatgagggccctcactggggtgtctctgcatggggaagcaggtagtactgtgagccaggagcaagtctggggttcctttcctttcctgcttccaccatgtatttagccttttttcacagcttcagcggtggcctgtgctctccaaggcactctggacatgtgtctgtccacctccccaagtgctggcttttagctaatgtaagctccattgggatgcagcatctgtgtcttcttgaaggaactgcggcatcttggcaagttcagtcattgattcttaggcaggtgggtctcagtgttttggcctctctcaactctgggaatgtctccttgccctgtgtataccacacaacagcaacatgcacgagagttcaaaanggcagctggggtgatacttac

SEQ ID NO: 217 - DNA, 546 bp, Homo sapiens
  misc_feature (521)..(521): n is a, c, g, or t
gatttgatatagtaggcactgacaaaagaggtcaaatgcaggactgatttctaagcttgcaatctatctaaatgcagatgctaatttagagtgcataattattacacaagtacagacctaggttagggcttaaatttcattatgtgcaataaaaaggttaagtgatggttaagcaaggtgtagattatccttacctctcctaatgacatgggctcacattggcattcctttcatacttttacgtataaagtgggcagttgtttctgaatgactgttgctggagcctcaggcctgctttgtgcttggtgctttccagactttctcctttattcctatcaccatatacgtactcacaaaagagcagtgtaaaccaggtattacatttccacaagggaaagtagaaccaggaatagccacatgatacttaccgaaagcttcactttccctatagagaaagcaagcaacatgaatcttctgatttggggcccagcctgctgctgtatacagaggttggcattatnggggatagaaatgtctgataggagt

SEQ ID NO: 218 - DNA, 557 bp, Homo sapiens
  misc_feature (449)..(449): n is a, c, g, or t
ggtgggaggtgcctaaaaccacaacgctaatgttatgttaatggcattatagtgagctgggtcaaattaaggacatttaggttgatttgttgcacctgtgcctaagttgagaccgtccctgctgagcaacatcctggcataagggcaagttcttaaccacatttcttcctgttagctgcagaggcggggtaggtgctgtcctgcagttgttcctgtgaacattgttcctttctgtcccttctccctagaccctccatctctatctgcctatccagcccctaactcctctctcagtgctttccctccagtacatacccatattttaattttcaagtgtgcaattttcacatttataattcgttaagaaagaatgcaagggaatgattcagatagtcatcaacacactgcagctgagtccagactctgtctacctttcttccctcttctgnttgcatcatctccaatagtttttctaggatccaaaactgatactgatgctgcccagtttttagtgctggccctttgaggntcactgattctcctacattttctggcta

SEQ ID NO: 219 - DNA, 504 bp, Homo sapiens
  misc_feature (468)..(468): n is a, c, g, or t
gtgaaatatggtcgctatttacagtttttcagggaaaagttatacttttctatgttaataaagagctgaagtggtctacagttaatgtgacatgtagggatgatgatatttttaaaatacattttgttgctaaaaagtttttaggccagtgcaaattatgcagtagaacttgtgttgcaaaaggaattataacccatactttaaaaatgcttaatccctcatattcaatttcatcaagccttgtatacttctgcttaaatgtaattcaatccttggttgttatggcaaacagaaacccaacaaaaagacagactctggtactatggtaacagagccactttatcatttggtcaaaatttggcattatgattagctttgtagaactgacctgtttatttggcaatgctgttaaggatcatttcggtttcagacttcaagttgattaataatttaatcttactttttatnactggaatcaaacgatggttggtctgtgtttctgtt

SEQ ID NO: 220 - DNA, 519 bp, Homo sapiens
  misc_feature (443)..(443): n is a, c, g, or t
gggagctggaccaggtgacagcggcatggacatatgccccaggctttcctgctggggtccatccatgagcctgcagttgccctcatggagccccagccctgccctggaagcttggctgagagcttcctggaggaggagcttcggctcaatgctgagctgagccagctgcagttttcggagcctgtgggcatcatctacaatcccgtggagtatgcatgggagccacatcgcaactacgtgactcgctactgccagggccccaaggaagtactcttcctgggcatgaaccctggaccttttggcatggcccagactggggtgccctttggggaagtaagcatggtccgggactggttgggcattgtggggcctgtgctgacccctccccaagagcatcctaaacgaccagtgctgggactggagtgcccacagtcagaaggacnagacaaagcatgggacatgaaattaagagtgaacttcttatgggaggctgcagctggatcanagaaaaatccagtg

SEQ ID NO: 221 - DNA, 490 bp, Homo sapiens
  misc_feature (487)..(487): n is a, c, g, or t
ccagagtgaaccaactgttatctgtaattcaacagataattaactgaaactcagtgctcaaattgctactgggagtttctgcagccaaggtcattctcatggtgctgcttgattttgtttctgtgtctcttatatcgttttaggcagggaggaacttggattgtccttcagtaaaattccactttgatgcgcaaagcgaacttgaatttctgcttggcctaccacaggcctgcaacatgcactttctcctcgaggtgagaaggtctttttctctccactctgaaacaccgctgcctgcaccttttgctgagtttagagtagcatcatactttctgccacgaatgccacaaatactgccttttcaacttggttttgtaaaatgtaatggtactaaatccgccaaagggaataaagcacccttactttggatttgccagtcccatgggtacataactgacctactctcccacccccaaccttgaaactncag

SEQ ID NO: 222 - DNA, 306 bp, Homo sapiens
  misc_feature (285)..(285): n is a, c, g, or t
gcaaggggaggtcacctctggcttttattcccttctccgcagggcccaggaactgcatcgggcaggcgttcgccatggcggagatgaaagtggtcctggcgttgatgctgctgcacttccggttcctgccagaccacactgagccccgcaggaagctggaactgatcatgcgcgccgagggcgggctttggctgcgggtggagcccctgaatgtaggcttgcagtgactttctgacccatccacctgtttttttgcagattgtcatgaataaaacggtgctgtcnaaaaaaaaaaaaaaaaaaaaa

SEQ ID NO: 223 - DNA, 489 bp, Homo sapiens
  misc_feature (467)..(467): n is a, c, g, or t
attgttgattatttgtgtagctttctaaggcacaaggtgcactttctgatacctgtaagtttctattagaatggtgatgtgagagaagtgggaaaagcatgttctttgactaggcttaaagggttaatgactaatactccagccccagggaggatcctgaatcggctttgaagttagacatgggtttgttaacaatctaacctgtatcatcagtgataaagggggggaatacggttcatttcacctgagtataaagatgtgtgaaagtgcttagcacactgtaggcattcacaactcgccttccaggacccctgtgagcaagctcatgaaaaagtgactgttggtagcacagcttgtgtcagtcctgagtatcctggctcttgtatcccatgcacaagactgctgggtgtcctcctgtattcactttactggttttagcctgaacaacaaccagagccaggccctantttcttatttctgacttctcac

SEQ ID NO: 224 - DNA, 474 bp, Homo sapiens
  misc_feature (465)..(465): n is a, c, g, or t
gaagtttgaagaggcaaacagtaattttgaaagccactactttgaaaccattctaaggcctgagttcccataggacacactcacataggcaggtacacgttagtcaacaattggaactgcctcttggatcactcagctgtgctttcatggctggatgatggaacactgtgcgaagagagatgggggccaggaagtagcgcttcatgcttagtacatcctccaaattgtctttgctggaggagaaaaccgtactcagccaaaagatcaggacaatatgacttgagtccacaaggacacaaacacctgagtagctgggcagcccttggcagggtctaagccaggaagtaaaaatgatctggcctagatatttaagggaactctaggaagaggcctaggtttttaaaatcctgtctctttgtcttaccataagaggctgagcctctcttcattttttgaaggccactngggtttctg

SEQ ID NO: 225 - DNA, 445 bp, Homo sapiens
  misc_feature (422)..(422): n is a, c, g, or t
gggatttcacccatgaattgcactaggaactgggagctcctttttgaacacaagtcccaagaggcagttgtgataaagtctctcttttggtgtccgctgtctgtaaatgaaacaatgcaaattggtgtatgttaaatgtgtcacagcttgcaggcaagaggttttgtttattctctaaggacttgctgactcgagggagcatcaaagatgagctttctgtctgtcttggcaagtgtgaaaataaagggagtcagtcacctccccaaaggtcatgtgaatcagaattcaggagaaagttggaggaaacaatggcagattccaagaagagatgcttgtttctccatttgagaaagccttactgaaagctactgaataaagagcacaatttccagttatttctttctttgcttttttttttttcnggccactataacagcacttccag

SEQ ID NO: 226 - DNA, 408 bp, Homo sapiens
gggatttcacccatgaattgcactaggaactgggagctcctttttgaacacaagtcccaagaggcagttgtgataaagtctctcttttggtgtccgctgtctgtaaatgaaacaatgcaaattggtgtatgttaaatgtgtcacagcttgcaggcaagaggttttgtttattctctaaggacttgctgactcgagggagcatcaaagatgagctttctgtctgtcttggcaagtgtgaaaataaagggagtcagtcacctccccaaaggtcatgtgaatcagaattcaggagaaagttggaggaaacaatggcagattccaagaagagatgcttgtttctccatttgagaaagccttactgaaagctactgaataaagagcacaatttccagttatttctttctttgc

SEQ ID NO: 227 - DNA, 296 bp, Homo sapiens
  misc_feature (243)..(243): n is a, c, g, or t
cctaatttgattgttcctgaacaaaccctacaccataacagcctgtctgaatgcgaggggtgctctggatcaggaagtcacagcagtcacactgctgcgattcctttaacccaggcatgcaggaactcagcctgggcccaggagacaggctgcctcggaatgagggagagagactccacatttgccatctcatatccgtgggttctgagcccacactgtcacttttagagtttcttgtgggtntatagatttatcgtgtggtgtttcaagctggntttcttttttttttggaaagt

SEQ ID NO: 228 - DNA, 421 bp, Homo sapiens
  misc_feature (236)..(236): n is a, c, g, or t
gattattacatttagcttttcattgaaccgaaagaaaaaaagtgatattggaccctggaaagattttgaaacttgagtggtttgataacccttctatgtattgtagggagaaaaaaaaaagtttattttattccactgtcctcccttaaaagcatcatttgagcaataaatgaatattgtctttaaaccaagggttagggaattttcctctctctctctctctcctctctctttcngttcaaanaacttcaaacatttgggaccacctggtattctgtattttcactggccatattggaagcagttctagttgcattgtattgagttgtgctggcagnagtttccatgcctgncaatgtatcatagncctttgttgcccagataaaaaatatttgatacgctttatggcgattttttttat

SEQ ID NO: 229 - DNA, 486 bp, Homo sapiens
  misc_feature (474)..(474): n is a, c, g, or t
gagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctcccggggaatcctctctgccaatcctgcgaggnctaggcccttta

SEQ ID NO: 230 - DNA, 500 bp, Homo sapiens
  misc_feature (472)..(472): n is a, c, g, or t
gaatgtatagaaaaatcaagcttagtaatactttaatattattctaccaatgtattttttttaattaagacttctgtatgcagcagggcatggtggcacacctgtagtcccagctactagggaggctgaggcaggaggattacttgagcccaggaattcaagcctgggcaacatagtgagtcctcatctcctaaatttaaaaaacaaacaacaaaaacttctacgtgtggtaaagggttaactcagcaagcttgggttgcttaaaccttgcacattccaaagaaaggtctgaccccagtgctgacctttgactggctactgggaaatgagctctgagcccctggataggaatgtttctgtttgcctgaggcctttgaccatgccagatagtttatgctaacaatgtgatttatagtgaatacctgttttcctgtgtctaaaaccctgggctgcactgtattaatttgacctngggtatttggggaagctgagactgagta

SEQ ID NO: 231 - DNA, 474 bp, Homo sapiens
gctcctccttccctcccatcgtcctcctccacctgcgcctcccttgtctgaacttcccaacgccttcctattcctttccaactccttttcccccaaatttcactttccttctcaggtctcacctcagccccccccttctccctgatttctcggcctctctctctgtgtgaaggggcctctccctaatgtctcctccttcccccctcttctctctcctgcggcccagcctccctctccctcctccattcctctctccctgcccttttcctgcctgaagagcagaggtgaggacctgggacccctgaggggcaggccaggaggagctcgggcgcaggccaggcccccttggtgaagcagaggctgaaggaaaggggtctgggtcttgtccctaggaattctcctccctcaggagattgggggttgggggaggcagcgggtgatggctctgaagctgaaccagggcctggctgtggc

SEQ ID NO: 232 - DNA, 526 bp, Homo sapiens
  misc_feature (523)..(523): n is a, c, g, or t
gaaggagtttgtgatcagtgatcggaaggagctggaggaagatttcatcaagagcgagctgaagaaggcggggggagccaattacgacgcccagacggagtaaccccagcccccgccacaccaccccttgccaaagtcatctgcctgctccccgggggagaggaccgccggcctcagctactagcccaccagcccaccagggagaagagaagccatgagaggcagcgcccgccaccctgtgtccacagcccccaccttcccgcttcccttagaaccctgccgtgtcctatctcatgacgctcatggaacctctttctttgatcttctttttcttttctccccctcttttttgttctaaagaaaagtcattttgatgcaaggtcctgcctgccatcagatccgaggtgcctcctgcagtgaccccttttcctggcatttctcttccacgcgacgaggtctgcctagtgagatctgcatgacctcacgttgctttccagagcccgggcctattttgccatctcanttt